SEQUENCE LISTING (18 sequences)

SEQ ID NO: 1 - DNA, 21 bp, Artificial sequence
  Synthetic single strand DNA oligonuclotide
ctctctcctggccagacacat

SEQ ID NO: 2 - DNA, 19 bp, Artificial sequence
  Synthetic single strand DNA oligonuclotide
agagaagaaggccacccag

SEQ ID NO: 3 - DNA, 18 bp, Artificial sequence
  Synthetic single strand DNA oligonuclotide
ggagtctggaatttcagc

SEQ ID NO: 4 - DNA, 21 bp, Artificial sequence
  Synthetic single strand DNA oligonuclotide
cctttgcctcaggaccatgta

SEQ ID NO: 5 - DNA, 23 bp, Artificial sequence
  Synthetic single strand DNA oligonuclotide
ggaattcctctgtgtgggccatg

SEQ ID NO: 6 - DNA, 19 bp, Artificial sequence
  Synthetic single strand DNA oligonuclotide
acgtcgacaggagctctgc

SEQ ID NO: 7 - DNA, 26 bp, Artificial sequence
  Synthetic single strand DNA oligonuclotide
ggaattccaggtttctgtcagatgcc

SEQ ID NO: 8 - DNA, 27 bp, Artificial sequence
  Synthetic single strand DNA oligonuclotide
ggaattccgttgctgagttttcgaacc

SEQ ID NO: 9 - RNA, 14 nt, Artificial sequence
  Hairpin ribozyme consensus sequence
nnnbngucnnnnnn

SEQ ID NO: 10 - DNA, 945 bp, Homo sapiens
atggggaaccgcagcaccgcggacgcggacgggctgctggctgggcgcgggccggccgcgggggcatctgcgggggcatctgcggggctggctgggcagggcgcggcggcgctggtggggggcgtgctgctcatcggcgcggtgctcgcggggaactcgctcgtgtgcgtgagcgtggccaccgagcgcgccctgcagacgcccaccaactccttcatcgtgagcctggcggccgccgacctcctcctcgctctcctggtgctgccgctcttcgtctactccgaggtccagggtggcgcgtggctgctgagcccccgcctgtgcgacgccctcatggccatggacgtcatgctgtgcaccgcctccatcttcaacctgtgcgccatcagcgtggacaggttcgtggccgtggccgtgccgctgcgctacaaccggcagggtgggagccgccggcagctgctgctcatcggcgccacgtggctgctgtccgcggcggtggcggcgcccgtactgtgcggcctcaacgacgtgcgcggccgcgaccccgccgtgtgccgcctggaggaccgcgactacgtggtctactcgtccgtgtgctccttcttcctaccctgcccgctcatgctgctgctctactgggccacgttccgcggcctgcagcgctgggaggtggcacgtcgcgccaagctgcacggccgcgcgccccgttttcccagcggccctggcccgccttcccccacgccacccgcgccccgcctcccccaggacccctgcggccccgactgtgcgccccccgcgcccggccttccccggggtccctgcggccccgactgtgcgcccgccgcgcccagcctcccccaggacccctgcggccccgactgtgcgccccccgcgcccggcctccccccggacccctgcggctccaactgtgctccccccgacgcc

SEQ ID NO: 11 - DNA, 2342 bp, Homo sapiens
gaattcaggggctttctggtgcccaagacagtgacctgcagcaagggagtcagaagacagatgtagaaatcaagagtgaccatccacgggattgacttggattgccactcaagcggtcctctcatggaatgttggtgaggccctctgccagggaagcaatctggctgtgcaaagtgctgcctggtggggaggactcctggaaatctgactgacccctattccctgcttgggaacttgaggggtgtcagagcccctgatgtgctttctcttaggaagatgaggactctgaacacctctgccatggacgggactgggctggtggtggagagggacttctctgttcgtatcctcactgcctgtttcctgtcgctgctcatcctgtccacgctcctggggaacacgctggtctgtgctgccgttatcaggttccgacacctgcggtccaaggtgaccaacttctttgtcatctccttggctgtgtcagatctcttggtggccgtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaataagcttttacatccctgtggccatcatgattgtcacctacaccaggatctacaggattgctcagaaacaaatacggcgcattgcggccttggagagggcagcagtccacgccaagaattgccagaccaccacaggtaatggaaagcctgtcgaatgttctcaaccggaaagttcttttaagatgtccttcaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcagtcatattggactatgacactgacgtctctctggagaagatccaacccatcacacaaaacggtcagcacccaacctgaactcgcagatgaatcctgccacacatgctcatcccaaaagctagaggagattgctctggggcttgctattaagaaactaaggtacggtgagactctgaggtgtcaggagagccctctgctgctttccaacacacaattaactccgtttccaaatacattccagtgtattttctgtgttgttcatagtcaatcaaacagggacactacaaacatggggagccataagggacatgtctttggcttcagaattgtttttagaaatttattcttatcttaggatttaccaaatagggcaaagaatcaacagtgaacagcttcacttaaaatcaaatttttctgggaagaaaatgagatgggttgagtttgctgtatacaaacaggtgctaacactgttcccagcaaagttttcagattgtaaaggtaggtgcatgccttcataaattatttctaaaacattaattgaggcttacagtaggagtgagaaatttttttccagaattgagagatgttttgttgatattggttctatttatttattgtatatatggatatttttaatttatgatataataaatatatatttatcatatttaataggataaattaatgagttttatccaagaccttacaaccacatttctggccatttaactagcactttataagccaatgaagcaaacacacagactctgtgagattctaaatgttcatgtgtaacttctaga

SEQ ID NO: 12 - DNA, 2062 bp, Homo sapiens
ggcacgaggcagggctgaagttgggaccgcgcacagaccgcccctgcagtccagcccgaaatgctgccgccaggcagcaacggcaccgcgtacccggggcagttcgctctataccagcagctggcgcaggggaacgccgtggggggctcggcgggggcaccgccactggggccctcacaggtggtcaccgcctgcctgctgaccctactcatcatctggaccctgctgggcaacgtgctggtgtgcgcagccatcgtgcggagccgccacctgcgcgccaacatgaccaacgtcttcatcgtgtctctggccgtgtcagaccttttcgtggcgctgctggtcatgccctggaaggcagtcgccgaggtggccggttactggccctttggagcgttctgcgacgtctgggtggccttcgacatcatgtgctccactgcctccatcctgaacctgtgcgtcatcagcgtggaccgctactgggccatctccaggcccttccgctacaagcgcaagatgactcagcgcatggccttggtcatggtcggcctggcatggaccttgtccatcctcatctccttcattccggtccagctcaactggcacagggaccaggcggcctcttggggcgggctggacctgccaaacaacctggccaactggacgccctgggaggaggacttttgggagcccgacgtgaatgcagagaactgtgactccagcctgaatcgaacctacgccatctcttcctcgctcatcagcttctacatccccgttgccatcatgatcgtgacctacacgcgcatctaccgcatcgcccaggtgcagatccgcaggatttcctccctggagagggccgcagagcacgcgcagagctgccggagcagcgcagcctgcgcgcccgacaccagcctgcgcgcttccatcaagaaggagaccaaggttctcaagaccctgtcggtgatcatgggggtcttcgtgtgttgctggctgcccttcttcatccttaactgcatggtccctttctgcagtggacaccctgaaggccctccggccggcttcccctgcgtcagtgagaccaccttcgacgtcttcgtctggttcggctgggctaactcctcactcaaccccgtcatctatgccttcaacgccgactttcagaaggtgtttgcccagctgctggggtgcagccacttctgctcccgcacgccggtggagacggtgaacatcagcaatgagctcatctcctacaaccaagacatcgtcttccacaaggaaatcgcagctgcctacatccacatgatgcccaacgccgttacccccggcaaccgggaggtggacaacgacgaggaggagggtcctttcgatcgcatgttccagatctatcagacgtccccagatggtgaccctgttgctgagtctgtctgggagctggactgcgagggggagatttctttagacaaaataacacctttcaccccgaatggattccattaaactgcattaagaaaccccctcatggatctgcataaccgcacagacactgacaagcacgcacacacacgcaaatacatgcctttccagtgctgctccctttatcatgtgtttctgtgtagtagctcgtgtgcttagaaacctcaccccattgattggtagttcgaagaattggcagaatcagttgcaataaactcagtcaaatgtacccagcctaccagagatggaccaacgatcctatgagagaagagagtatggtgctgggtccttaaaaaaaaaaatgatacttggtccttaaaaaatatgctctcccctccctttttaaacaaatggcttgttcagtcacttgtttgtgtttgaattgatttttaaacagcaggttgtgtgtgtgtgcagtgatgtggtgggagcacagctttcctgggtctggattcccgtggctttgtgcttatgtcatttcttctctctgtgctggtgggggcctctttaccatagcttaagaagtatccctgatttattctggtgtctaataaacacagattatttgtaaaaaaaaaaaaaaaaaaaaaaa

SEQ ID NO: 13 - DNA, 1203 bp, Homo sapiens
atggcatctctgagtcagctgagtagccacctgaactacacctgtggggcagagaactccacaggtgccagccaggcccgcccacatgcctactatgccctctcctactgcgcgctcatcctggccatcgtcttcggcaatggcctggtgtgcatggctgtgctgaaggagcgggccctgcagactaccaccaactacttagtagtgagcctggctgtggcagacttgctggtggccaccttggtgatgccctgggtggtatacctggaggtgacaggtggagtctggaatttcagccgcatttgctgtgatgtttttgtcaccctggatgtcatgatgtgtacagccagcatccttaatctctgtgccatcagcatagacaggtacactgcagtggtcatgcccgttcactaccagcatggcacgggacagagctcctgtcggcgcgtggccctcatgatcacggccgtctgggtactggcctttgctgtgtcctgccctcttctgtttggctttaataccacaggggaccccactgtctgctccatctccaaccctgattttgtcatctactcttcagtggtgtccttctacctgccctttggagtgactgtccttgtctatgccagaatctatgtggtgctgaaacaaaggagacggaaaaggatcctcactcgacagaacagtcagtgcaacagtgtcaggcctggcttcccccaacaaaccctctctcctgacccggcacatctggagctgaagcgttactacagcatctgccaggacactgccttgggtggaccaggcttccaagaaagaggaggagagttgaaaagagaggagaagactcggaattccctgagtcccaccatagcgcccaagctcagcttagaagttcgaaaactcagcaatggcagattatcgacatctttgaagctggggcccctgcaacctcggggagtgccacttcgggagaagaaggcaacccaaatggtggccattgtgcttggggccttcattgtctgctggctgcccttcttcttgacccatgttctcaatacccactgccagacatgccacgtgtccccagagctttacagtgccacgacatggctgggctacgtgaatagcgccctcaaccctgtgatctataccaccttcaatatcgagttccggaaagccttcctcaagatcctgtcttgctga

SEQ ID NO: 14 - DNA, 33654 bp, Homo sapiens
  misc_feature (26)..(26): n is a, c, g, or t
gggaatgagtctccatttcttctgcnatntgctgaggttgcctcctcggccagagaacataaaagtgttattcagtaactctctccttttcccttggatgtgggaaggaacgggcgcaccacctttcctccctcttccatttccatgctgcctttttgaagttgcaagagatgctaaaagaaggtccaaccccaagtgagagcctggcctttgcctggaggggtaggggcaggtggcctatatcgccagggtcctggaaaggaacctcttacccactcactcccaattcctatccaactcaagtcactccagtaggctgcttttgagatgcaaatacaaaatgctttcttctccccctctactttttcattgaaatgctttgcatctcagaaaagaggaaaaaatgatgtgtttaagacaggagtgtctcacccgccatcaagcctgataaggaattgttaaattttaattagacaatttgattccttcaagtgccgaactttcagggaaggcagcaccaagacttaagctgagacggaagcataggaaacacgttgctggggtttatgctgcatccagggcttctgtcccccacctttctagctctgtgccccagatgaaatagaaatgaggtggtggggatagagggagggagagagagagagagagagagagagagagagagagagagagagaaagagaggatcagagaaaatcctctctgttctcccaggacctgccagctttcttcctggatcccaggctgtcctaacagattgacccttttcaccccactccatcagcctggggactaagtgttgctacaccttccaagtttctcctgagagaaaatgtcaggtttgggtgtcagcctcaaactgctgagccttaaaggagcttttttcccttccaatacactttataggttggggcgnctgccagttttggggcgtgactgggaaagcagncccttccatacctcatctataccccagaaaggactgcccactccctgtcccctcctctactcctggggggcagtgcagtgggtggcctgagcagggtggcctgcctggggcagcctgggtgggctagcttagccccgggtatctgggctcttccagtacttctcaacttgagagctagctaatttccaaattagggcacgcacagaaaatagtagcatttgtaaggcatatagggtgaatagcagaagctgctgaggttggaagtgcctgcctctacatcccaattgacctaatatttctatgcaccttgatgcattctagacctctgattggggaagttccactctgaagcttctgaagagagccactattatagaaggctggagttaggctagatgttaggaagaacttgacatgcaggggtcctgagcactagaatggatgactctgaaagatatttctgtagatctttgcccttctgagctagggcagagcaaagagtcctggagacagatgaggagatctttggctctttgatgaccagtagcttgcaggtcaccacacaaatgcagtcttctctttctcctggcttccaccagggcagctgagaaagaaaagcctaggaggcctcagaggacccatggaaccttgtctgcaagcatctgcatggtgagggtggaatgaggagcagcaattacccccactttcctgcacagaaaggtgccttaggggagaggcagccacccctggagtgaggcctggcagctacaggattagattgttcatcctccacccaggaaacactgtcaagtagctcaacctgtaacagctggaaaataacagtgtccatgcatgtatttggtgctcactaagttcttgttgactggggattgtgatttggagttttgtaagcttttatgcatgcccaggggccacctgggagaaagatggagatgacatttggagggtggtgagcaagttgatcatattttcacaatcaaaaatcaaaatgcttgttttgataacaatagatttttaaatatactgtgccactttatttgtctcaaaaaaatttctcatgtaagaataatgcaccttgggttatatatttagccantctccaytttaaaaaaagaacaagaaattgggaccatcatggcaaatacaaggtgccatgtgttatgtggctttgcattttccctcaacaagctccagagtggacaaagaactcaccaggcttctttggagccagaaccagcccagcgtaactggagcagcactgagaacctttcctcactccgctaccatgaacacaagctccagccaggatggagaagctactcacttgtgtattcttttgggatgccccttggagcccaacaatctgtgcagcccggagacagaaggacaaaagggagggatgggctggagctgcagcagggaagcctggggaccagcaggttttctcaggcctcttgagaaaataccagcttagaaatgctgggaaggccctgtattgctatccactctacaatgcacttcccmctgcccccaccattttaatgtctctgaaatcagaatgcatttkacaatggatgacatcttggagctctactggggttggatggcattwttttctttcctagtgatatatraaatagtggtgcatcgtcattcgattttgtcttagagtctatgaaatgtggtgattagtatgacagtcatcgrctatagcagctgggctgggagagctgtatcttccaggccagctgctacctcatctcaagggtcacttgagtttgtcctacagccagggcccagggacaggctttgtcttttccttccwttacttcctcccaaggagttggtggctgcatcttgaaatccatctcccttcatctagccacctatttttcaggatgggccataggaggcagtgggcccaggaggacctcagaccctagtgagctgcatttgaacagcaacagtcacttcatcttgctgagccgttggcttgtggtctgattgcttgtttcatttcattcttttgcttggtttgatttttaaagttgacattctcaagggcttacctgtgcctggcacatggccatgagtcatcttacttaatactcacaattgttgttatctctattttgtgtatgggaaaactgggctcaggaaagccaaataatctggctgaggccccaaagttagtaaatggtagagwtgggacttgaacccaggttttttagtcctcaactcgaatgttatactagcacctctcaggctagagtaccttctctgtggcagtggatttgctgatgtctgaattcctgctcagcttgatgtttacatggtgagagccagctaggctgcccctgcttgtgagaggtgaaagccccaagtagataaggtgctgaagacacgatagaaaccaggactgacaccaggtgttaaaagatagccctgtactggccgggtgcggtggctcacgcctgtaatcccagcactctgggaggccgaggcaggcagataatgaggtcaggagatcgagaccatcctgactaacacggtgaaaccccgtctctactaaaaatacaaaaaaattagctggacatggtggcgggcacctgtagtcctggctactcgggaggctgaggcaggagaatggcgtgaatccaggaggcggagcttgcagtgagccgagatagccccactgcactccagcccgggtgacagagcaagactccatctcaataacaaaaaaacccccaaacaagcaaacagacaaaaagatagccctgtaccaaggtacttataggggttacaggtatttaggcatttgccatgttttcccttttttttcctatggtgtaaatatactacgtttgtattagcaacaaaaagcaacgagaacaaccaataataaataatagattagaaaatcaaatgtgtgaggcctttctcagaacttgggtacagggactctcccttaccctgcagaagtaaccagcactccagagaatgaatgctcaccctcaaccccacctgacctgcagtgctcacatttgccatctacattgtactcataattttaaatgctagactagtgcctgtaatcccagcactttgggaggccaaggcaggagtatctcttgaggccaggagtgtgagaccagcctgggcaacatagtgagacccccatctctacaaaacatttttaaaaattagccaggcatcgtggcacatgcctgtatttccagctactcaggaggctgaggtggtaggatcacttgaggccaggagttgaggctgcagtgagctatgattgtgccactgcactccagcctgggtggcaaagtgggattctgtctctattaaaaaaaaattaaatcccagtttacatcctccctcctccaggaagttctccacaaacgccccagccacacacatgtgcatatgctcctgaacatcaactgtagtggacacggtggagtgccacccagatccccctgcaggaccacagccctcatccccaactactgggagtgttggcaactgacagtttttggcaattgcccactgctgaggagagatgcctctttcaaggcctcgctgctttccctgggacagcagaagcttgtgacatctatgagaggggattaaaggccctaccctgttgccctcatttaggcaactggcagggtgccctgtggggtcacctgatgcctttcttgtgacttctccctctggcctgtcctgcttcctcacttctttgcacatcttgatcctgagagcattcccctgtacatttcctgcaggctgatttcctcttcagagtctacttctagggacctgtattagtctgctctggctgctatgacaaaattccacagattgggtggattgaacaacagaaatctgtttcctcatagttctggaggctgggacgtcccagatcaaggtgtcaacagggctgattttgcttaaggcctctctccatggctagcagatggccgccttcttgctgcattcccacttggtctctcctcttgtgcgcgcatccctgatgtctctttttatgtccaaagctcctcttataaggactccagtcagattgggttagggcccaccctaagcgtctcattttaacataatcaactctttaaaggccctgtatcctaatacagtcacattctgaggtactgggggttaggacttcaacatatacattttgtgggttgggggaagagcatagtgcagactgtaacaggacccaacatgcagcacctatggcacccttggggtgccttactttttcatcacttgttaatctggtagcctatgttagcaggtgcctttcaagtgggaaggggttttcttcccagttgcactaacagaacctttgattcagttcagcaaacatctgttgaatacgtactgcccacaaaccagagtcaagagttgggaaagaacaagatagtatggtttttatttgttcattccaagaacaagaggaggaatgagcaagacatgttgtaactcttgtgatgtctacaccccagtgaaaaaaagagactcctaaacaggtgtctcctgcagcactggggtgtgtttcagtgctgcaggaccctgaggagggtgggtgatggctcaggcaggaggatatgggtggattctaagacaggaggattctaggcaagactcactttgaaggatgcatagggactggaaaagcagagggaagggaagtgaacagaagagttttccaaatccctttcttattaaagtagggataataattagacaagatcaagcatgttccaggtagtatggccatagacagggataataattaaaagcctccgagggttgttttgaggataaatgagacagtatatccaaaaaacttagcaaactatcaaaccctttacaagtatatgatgctatcatcagcagcagggagtggggggagaagaagagagcgatggtcaggatgggggcagtaagtgtgcctcacacgcctcaggcccctgacactgctttgcacagtgctttatgcccagcaggttcaataaatgccgattgagtaaatgaatgtcagaagcagccccaggcacctgatcaatacatacatgcaggcctcccgctgtgggctccaaatgggaagagggctcagttggttttgcagaatacagcatcaatatgacatatgcttcattaccttagaacaggaagatttgctgcgtggtggcattccatgtcaccccacatggaattaggcactttgcttttccccgaggctccagagtgatggtggttggtaggacatgatccatattcttctactaaagctggaagagtgggtgtgtcagacagggatgagagtgtagagattgggcagaccctcttccaccatcctctctgacccattgctggaggtgtgccctgttcattgattggacatggcaccacattcatcccaaatggcatcaattggtggttttcaatttgcagtagcaaagtacctggaagtcatgtgctttgtatgaaacgccttggaatgctgataagtttaattctattctgtaaaagaggaagacttttgttagctgaaaagccaactatattatccatgcattatgcttcagtcacaaccaaaactccggctgtcaagttcatcaactcctgatgcctcccaagtctgctcctgagcccctctggtcccttttctctgtgaatatgcgacaccagcctcccagagccccacgcagaaatcctagcattaccctcaactccttatctccctgcttgtcccatacccacttttgccccacacctatccaaggaccaaggaccatgaaattgacttgcagtttagctcctaattctgaacaggtctctccatcctcgtttccactatcttcgttcagatgaccacaatttctaacctaaattacagccaaaacatcactctcctctgagctctcttctacaccatacactttctagaacaccagtccaatagggtcatgtctctgggcccaacctctcagggactctccagctgacaagacacaatctggcctgtaaatgtccctgcagtttaattatcctcaacattactgccataccctacatttttggccaaacagaattgtttgctgtttgatgatttgataccatgtcatattgtctgtctggaacatccccccacccctctcatttagcaggctaagtcctccttctacttcatcccagatgatacccacttcaggaagtctttcccttctgtgggatgagatgcccattctgcagggctttcactccctgcatcctgccaaacctcatcatctcttacctggattcctgctacagcctcccagttggtgtcccgcttccactctgggcccctcctctccgttctccacagtgctgtcagaatcacctattcaaaaggcgaatccgatcatgtggttcctgctgcccttaggatcatgtataaactcctagcatgacttttaaggccctctatgatcttgcctattgcaacctccccagactcaacccttgccaggtccctctgcatcagctatccagaatctctttgaggccctccacctgctgtctacctctctacctctgtgctttgcatatactgcttccaatgtctagaccttctgctgactcctagttctttacctggctaattcctacacatccttcagttgtctgtctgttgaacatcacttcctctagaaagccttccctgaatacctgaactaggttatgtatctctctccctgttccatttcctactccctatcacccttagatgtaattgcttgattaattggctgttgtttctcctagaatgtgagctacagaaccatgtatattttgttcctgcctatatttctagaactatatagaacaatgcttaataaatatttatagaatcaagaatgaatgaatacccatttctgtttcgatgactagaaggtagcaagcctggttaactgaagtgtgtggtgcatgggtcgtgcttaagaagtgtttgttgaatgaataaataaatgatggatcagctctgttcaagctcatcttattatgcatttttaaaaaattgtgtttgctcatttgtcctaccagctgtaaaaacttgctagcaaaagagtggcagaaggcccaacatttttagaaaattcttagccgtaaaccttaaaatcccgagttggaaaattctcattattaaatgccaggagttggtcttcttcctggagacctctgcaagaggccctctcactgacaccttgtgtccatttttcctggccagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtaggtgggccccctgcttgctccagcactttctccagcagggccctgcactggacactggggactctagctccccactggcttttaccaatgagtttcctggtgcttccccaggtggtttttccttcactctgggcttactttttctccttacgaaatgggtagattgtttccttaataatcccaactaccatttgtaaagtgcttactaagtgctgggcctcacgtagggactttaaatatagcattttcctatataaccctcacagcagcttagaggctggcattattgccaccattttgcagttgataaaccaggtggtcagagatgttaagtaactgctgcagcatcacacggctggcaagtccaagctggaattctggcctcagagttagttccttaggtcatattggaaataggagtaaccggcaaggatccccaaggaggggcatgtttatgctcccaggcccctgaaaccttcactcccatctccccacttcagaaatgggtctgctgctttatgctccccatatcccctctccttcccacagcttatcctggggccctgtccaggacctgcaggtagaggctgccatggactgtgctgtagcccttttgttaggaagaattgtgtagtcactcatttcttggcccagctctgcacctcaaaatggggagtaggcagcacagtctagacactgtgcctggtgcattgctgacacagacccagtgaatactcttggtaaccctgggagtccatgctactcttctcactttacaaaataggaaacaggccgagcacggtggctcacgcctgtaatcctagcactttgggaggccgaggcaggcagatcacgaggccaggagttcaagaccagcctggccaacatggtgaaaccccatctctattaaaaatgcaacaattagccaggtgtggtggcatatgcctgtaatcccagatacttgggaggctgaggcaggagaattgcttgaatctgggaggcagaggttgcagtgagctgagatcataccattgcactccagcctgggcgacagagcaagactctgtctcaaaaaaaaaataaaattaaattaaaacaaaaacaaaataggaaatagaagctttggagaagttgcctttcttcacccaggtcacagggagaaatgctgatggagaaatcccaaagcagatattattctccaggaagacaccttcagagccagcgagcagatgtgggagcatttagggatcatcggtgggcacaagctggatcctgcccctttctctgcccagctttctaggatggcatgtccaggactcaagcaatctgggagtctaggtgcatgggaaggcatgggatcaggcatttaggaaagtgccggtgctctgcccactgaaccgttaccttccttccctgagcaggagaggaattgaccagtgcctctgaggccatccctgcctgagagggaaggggttgttgaaagaaaatgagaaagctttgtaggtttaaacaggggagaaatctagatgaggacgctcaggtgaggaggcgagactggtggaaagtggcacacctccctgtccctgggcccccaaggggctcgtgcgcttactgttctcaccaccaccccgggcccaagggagctctgatccatcaccccttggcttcctttactacagccaatgcaggctgcaacttccaaaatgacctgactggatattaaggaagaaccaaaaggaaacacaaatacaaaaccacataaaaatattcagttgacacaggccaggagtccaattccccctggatccatgtttatgagggsccctagactctgtccatgccttcttaattcttacgcttgatcccctccttcttccctcttccaaaggagggtcctaggtctcagaccatagggraaaatggtagagcaaacaaactctctgtgcctcagtttcctcatctggaaaatgcagataataatagtatcatacatcatagcattgttgaggatcgaattagttgatgtatgaaaagtgattagaatgatacatggctcacagtgagcactgtgtaaatgtcagccatggcgatgatgataaagatgaagatgacaatagacatccagcactgctccacatagggaggactctgtctttcctattcaccgcccccttgggataaagaggaaggagagagtactccctatgttcgccccaggcaagtaggtaccctcttcccagaggaacgtgtgttctacccagggctctgggcatggcttctgttagcttccagaccccctgtccctacccaggcacttgggtagggacaaatccatacatgggctatagggggttcccaggaatctgtaagtctttcgaacagatcctttaaacatgcttgttcaccttattttaaaggtgaagctgagtgtgtcaggggaggagggtgcagaagccattctcagagggcagggstcagacttccccacagcatctcatcaaactagagctgggcactgagccttgtttaatggacattatctcagcgatttgttgtccctagagaacgtcccaggtgacgtctcaccctgccctgatgtcctatcagcgcaccctcacaaccacgcactgtcccctctctaatacatgctcttgtcatccagattttttcctcctgttccatttccagcaatgccatgccaacatctgtctggcttaggactaagtgtgcgttatttatcttgctaagtgtatgaaaagcacgctggttttggagtgagaggaccaggttcaattcccagctccattcctttccatctagatgctattggaagagttcctgatgtccacatgtgccattccatgtcagcctcacaaccctttaaggagcggcttcctgtgtgcatttattttctagatgaggagattgagaagatgaacaaatatctcagagttattcattcactcagtcagcactgactgagcactccgaggtcttaccccaatccttgtgcaatgggctaaggcactgacctgctggggatgcagaggttgacagggcccctacagtgcagtctgctgccggtgacagtggagcagtccccataggaggtgtggggcataaggcccagcatgagggtgtcagggaagactttcagggagaaatgatgctttcggaaaaattatgcaggagagggagggagacacttgcattctcaagaaagccacccagcagggagagggagtagctgbctggaggtatggaggagaggtggttatgtcatttgcctctgaggcttactgtctgcattctaagatataagcatcaagtgtttggaacagtgcctgacacatggtaagtccttagtattattacagttattaggacttagctgagccagctcagggcctgtactgcaggtctcagctttatgtgagcaagagcattaaggaatgatgcctggatgcctgggggtgtgaagaaaagagccttgggttcgactagggaacctggggccactccttcctctgctactaaatcaccaagtgatcttgttctgttttcttctctgaccctccctagttttgtccacccttgaaataatcatctttccttttcacatttcatgcttaccaagtacttgtcacctaattatctcctctcttgataagctagatggtyccttccagggcagcttagtagagagcatgggatgtgatgtttcagattccagctctgctgcacacctgccaggtgaacttggccacgttacatggcctctctgggcttcagttccctcacctatgagtgggataagcaagcccttcttgtaaaagttttaagaacaatacatgagataaagtgcaatgcccacagtagatgtcttatggacagtggccaccaatgcattttcttgagtctttaagttagcagccctaactactctccaaggcagcttcccctgggaccacagaggaaatctctttgttattctgggctccagataggccagtgcaaggagagatttaagatgtccatgaaggcaggactgtgtctgaattgctcacaactgtgtccaggagcctaggatagaggctggaacacagtaatttgctcaataagtgtttgtaggaaagaagggatggcagaaaggagggaaggagaggaggaagacagggaggaaggccctgatatctcaacctagatccccagtggctcccaagtactggtcccaggaggggcttcaaagtgggatgtaaatcaggtagtgctggttttctgcccagtggtaccctaaaggcacactctgtttatgccaggaaagccctttacgtaccctcctgcctccacccgcaaaatgggcatccagctgttagctcctgccaacctggtcagcgcagcagcacagggagctgggagagaggccctggtacggggcccccatgtgagctcccaggaagacagtgccgcatgagaggcttgctgtgttctggggcaagctgctgtttctgccacacaggatgacagcagctgcattgcccatcattgagcttaaatgccagcatttggttggatgcctaggagcagaagagagggcagtgatcagaatgagcaaggttataattagccctgctgccaagctacacggtgacacgaagtctgccctactccgggttgcgggagggggctttcacgctcctctctgctcctattggtagaagccaaaggtgcatctccctgtcctgactccatcctcagggaactggggctgctccaagcttccagagcgctagggacagctgccgtgagtgtgtgcctgcgtaatctgccatgagtgtgtgcctgtgtgtgaatgggtgttacgtggggaggggggagggcggtgcgttcatgtgtgtgtgtgtgtgtgtgtgtgtgtgtgtgcctgtgtgcatgtgtgtatgagaaacgtgctcctgccaggagctagactcttcatttcccatcctaccaaggacacagctcatcacagcaacccctccgcctaccccagcccactcgggcgttcctcgagcactagcccagatctgccttctttcgttctctgggtataagaaggagcagaaatttcttctctgtttcatggcgagtcttcatcttcaccattattcttaacagatcattttggggccccgactctgccatgtactgggccaaccactgaagggcttgcctggctcagccagagcctgggccagagcaggactaggaactgagcccacaggccttggggaaatcactgcactttttggaggcttggtttcctcattggttctcagtttcctcacgggcattctaaaattttacctggaatctaatttacagaggtatggctgagtgataacacagggaagccagtgccattgggaagagtttaacgtgatacacaatgtccctagaaaccagatgctcagcccgccatgtggggtgcagggggaagcagcaggcttgggtcaagagtcagaatcaggggtgagggaaagcccaggctacagccctgttggggctcccacaggaagtgcaaggcagggcagattaagcaacctagggttggctagtctcaacatcctggcaggctttgggctacaggggtgatctctagttgtctctctggtacctgagataatttgggacaggggaaatattgtcttggtgtgtgtgtgaattagagaaacggggtggctggcttgcatttgagaggcatccttccaagtcagtcattttctgtcttcaggaattaactagctctgggacggtaagtctctctctgggtctgtgaggctcccaagtgccagaatatcaggatcagagaagatagaaaaatatagtcaatataggtgtctctgtgatgaatgggtgccaaatacacaaatacagaatctaagaaaacacatggggttaacaaaggttgcagagattaaggtttaaagttgaggccctccgtataagttggctgtctttcttctagcacagtaattggcaataagtggtcttatgtatctgggagaagataagtgagggscaagggcctcagacacccatgggcatcactccccacaccccagctggatgcccacaagacttgcagctgcctcctgagtctgtggcctcatcggctccaggagtaccaggctacaggacctaagctaatctcccactcctgctgtccatccattatgttgctttgtccccaggtggtaggtgagtggaaattcagcaggattcactgtgacatcttcgtcactctggacgtcatgatgtgcacggcgagcatcctgaacttgtgtgccatcagcatcgacaggtgagcccagccagggtggaagaggttgctctggccactcaccattctggctgccccagcttttctcaaagccaggctgtgtctcgtgtctgtgatgctgtgcagctggctgtgtgcatgggggtccatgtgtttgtgtgtatgggtgtgcatgtgtgtatgtttggctaggagagcacacactccctaaagagaagccatctatggacagtgatgctgccaagcatactcagacagtgactggtacaaaaggggaaagattgtgattctgacggaagccctagctctagcacatcttactcatgtgaccttgggcagatgatggacgttttcagagctctcagtttcattttctagaaacctacattgatgacacctgtggcctggagctgcagtatctggttatgcattttatgtccttggcacatggccaagggtcccaggccatgcacaccttgctaagctgcatccttggagccttccactaatttgcacagatatgaccctacctccctcacaccattcttggaagatgaaataagttaatgratgagaagggctgggtaaaaaaaaaaatgtgatgaaggattgtagacaattgccattgttatttcctgctaactaaggcttaaatcttgctctgaggggcctggctaacaatttatcaacaaacatttccaccttatgggctcaacacaagtgcagtgggtacagtttcaaaagtgcaaagatgcaggccatggttcttcgaaaagcctgaaatccaggtgggsccggttagaataatagatatgagctaagagtaaacaatagctcaccagtgattaagtgtttgggctttgccaactgtcctcgccatctgagtttggggacaggagggagggttgagttagcatctcaggcaagcttcatagaggtgtgtcaactgtttagccccaaggagtgagggtgtctctggtgtgtgcatattgtggagtgaggggtccctgggcctgcaccccagattcagggtcccccgcccttgcaggtacacagctgtggccatgcccatgctgtacaatacgcgctacagctccaagcgccgggtcaccgtcatgatctccatcgtctgggtcctgtccttcaccatctcctgcccactcctcttcggactcaataacgcaggtacattctgcttttgtttgcctgaggctcagctggccctgggcccctgctccctcgaagggccctgagggagcagagtccctggcacagatatgggtggaggcccaatggaggcctatcactaccctggactgagtccaggctacgtgtcctgggccaagccccactcaagagttgttctaggttggcagagaggaaacagtggcccaggacacaacggagttggatgaggagtggggataatggttcaggggtatgtctgggaattttcaactttggttattaagttgaagaggcaagacgtgcttcacaaactgcatgtgtgtatatgtgcacatgaacatgggatggcggaggctactgggcttccatccactcctgacaatagtatttttgtaatgaagctgcagccctcagggcctgctaaacccaggtcaggccttagccacctggagcaggaaagctagaagtgaataggaggtaagctgaccacatgcccatttctgtgggaggcagagtcaggagtgtctcagacagaggcaacacaaagcccccgccagcgtggaacttcctctcatcagggaggcaagagcgatggcaggtgtgcacctctggagatcgtaacaacaaaagatacagtgaagccttgaaaatgtgtggttcggtatctattataagcatcacaggaattctaagtgggaataacgctaaacatacttggattaatgcacaaggccctgagatagaggacaggcgtctggtagacctagaagggcacgcaaacatatgaaacacataggaacacaagtgagttcaacagacagagccaagttatcttgctgcaaacattaaaaggtggccaacctctcccaatacacaggtcagactaaaaagatggtttactcttttaaaagttttcttgtgtcattctttctggatacatcggcttcacttgttatgcccagacatggcaaaactaatgaccaagtaatgagggaatagtaatggaaagacttgggagcagtccatcatcacagcttaactttttgctcacaaccgtgtttttaatactctggtatctgctgtgcgtttgtgtatatctaagatgaccaggcagccttaaacatctagttgcgttcatattctctgtaaaatcgctccttgttcctggaaggacatgaatgggctcttgtggaattatggccggtgggcccgctgactccctgcctgcccgggctctccctcccccagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggtctcaagacaccccccaaccaactccaagggtccccacctaaccattaccaagagggctcatcttatgctcaggtgggggcttgggaaacctcagcaagggttaggtctaggctaaaggaattcccaggagccgggcaagggcagattttgaaggcatggacctcagtggagcaatctgagtctccaggagagggaggcagggtccatgacactaaataacaaggggaagtctttgcttggagatctttgtgactgaaggcggcaactcttgcttggtacccccgtgggctcctctcttcctcttttctggtttctctgtctcactttagctccctctgactcctccatcctctttcctttaccttccgtactctctgtcctcccctccaaacacacatcacttttcctgacttcctctccactatgtctctcctgtgcttctctttcatttcccccctgatgtcttgtgaattctccccttcactcagtccttcacaaggaagacagtgtgtgggcacagaaggaacaagagctcttgggctagacgcatcaggttcagatcctgtcactgacactttttttgctgagtgaccttaggcaagtttcttaccttctatgagcctgtttcctcatctgttaaatgggaatcaaaataccagcctcacagggtggtcttgaggattccacgtgagaagggacgtgagtgtgcctagcacagtgccaggcccttagcaggtgctccataaaaaaccaggtccttgtgttcctcgtcatacccaccactttttgtctcattccagccttcccccttgccccaactgcctcctctggcccccacccttctgatctctgagcccttctgcccagtctgagttccatggactgctgcactttgggtttctgtcccccctccatccccaccactttgtgtgacccatgtgctggctcactccacagggcaactgtactcaccccgaggacatgaaactctgcaccgttatcatgaagtctaatgggagtttcccagtgaacaggcggagagtggtaagtgctcaggccaggacccagagccaggtctttctgcccctagggaagcccactggccatggttctgagacctcagaagctggccaatgggagaagcaccccagaaacccccaccttgcctcagctgaaggcagactcaccgtgcacacctccaagcaggcatgaagtgagacacctcggttctgcaaggcatggatgtgtacgagaaaatggttggccataccaacgtaataaaaatgataataatggctattcacatttctcaaacatctaccatatccctattatctcatcaaatcctcaccacgaccccgggaggtaagtctctttgatcgaaccgatcttcagttgacagaagaggaaacaggctcagaaagattaggcaactcacccgtctcaagagttggtgacactaagcccagacctgtgtgactctgaaatccacacctgtgttctttccactgacatgagctgccttatggatgggcaggttctggggtaggacgagcagagcagctgcggggactggtggcggassagtttgtgtacatagagccctcaggtgcggaagcmmagcagaccccagcctctgccaggtggtagctgtaccaacatgcaagcagcaggcattccatcctccagagggatggagaacagggccagagaacccacagagggccgcatacaaaatccaggtctggtgtcctgccttcacctgcactgcaagggcaggactctaagaagctgtttatgaggcaggtgccaaaacagagcctcagagtcagggccaaggcagcagcccagtcatgccacctaggcacatagtgaggctgcactttagaagttcagactaacacctccaaggcctcaaacaagagaacctatggaagaacccaggaggccatgagtggatccatgccagggctctctaggtcaccccagcagggaagatgtggggccccaggggtcagccttttggcacctagattagtctatccaggagaatatggagcccacgtgtgtacgcaggtgcaggtacccatgaagtgggagcactgggccccttgtttgacagggagaacaggggtgggcaccctctttgcagtcggaacatgagtttctggaggcagggccaaacatcatcagctcttgaccccagcagccactgtggcacctggcactttgctaacagtaagtgttgctcaggccatgagagacaagtccctggtattcagccctggcagaacagaagtggggtattgaggctgcatgaggattgccatgggaaaaaggacaggggcaatcctgcaggggctgccatgggtcctgggtcccatgcctcagtgacatccttgcctccctggcagggtgaccctgtggtgtttgcaggagtcttcagagggtgaaagggaggggccagtgagatgggtggctgatgcctgggaacttgtccggctttacccagagccctctgcctctggtgcaggaggctgcccggcgagcccaggagctggagatggagatgctctccagcaccagcccacccgagaggacccggtacagccccatcccacccagccaccaccagctgactctccccgacccgtcccaccayggtctccacagcactccygacagccccgccaaaccagagaagaatgggcatgccaaagaccaccccaagattgccaagatctttgagatccagaccatgcccaatggcaaarcccggacctccctcaagaccatgagccgtaggaagctctcccagcagaaggagaagaaagccactcagatgctcgccattgttctcggtgagtcggccctggctgctggccacagcccggtctgtgaaaggtcccattccctgccatgtccttcctaaccatggctgcaggatcatggggntagcatttcctcaggcacaggccaagcccattgacatacagacagccctattaggtagattctgtaattgtgctcactttacagatgggcaaacagagttttagagtggttataaaacctgaccaagaggacccatggtttgaactcaggcagtctgactccagagtctggaaatttgactagtctattatgctggcaccagaacctcatgggcctatgtgcccaggaaagtcactttctctctctaggctcctcatctgtagagtaggttacttctcagggctgtcatggcaatcagttggtgaaagcattttctaaactacaaagcactatccatatgtaagtgtcactattatggtttttattactatggctctttttgaggaattgggaaattcagttcacttgcactcaaacaagactccatgggaccagagctgtgaaaaataaatgagatgttggggagtggcccccaatctaaatgaaaagcatccctgtccataaagcaggatttgtcaatagaagtctagaaaaccacatagtaaaggtcaaaacccaaaaggtggactgatatgggtggctgggaaaaggagggtcccctgtgactctgtcttccaggaagcacctgaatcccctttgactgccggatgatcctggggcgtgagtggggcagctgttacctcccttcttttatgaatgagagcacttgatcccatgggactactcacgatcatgggactgggatatggtaggctaggattggagccaggccctctgctgcagtccataggggctcagctctgtggccctactcttcccagacaccattcagtctccagggatgcctcctgtgcctgtagggcattccatgtacacattgcttccaggctaaatatcacacctgtgcaggaggtcctggaaaaagtgcacctgagttaggtgagcagagcaggtaggtggctgccctgggtgagagggcatggttcaaggcagggactagcaagggtgtacccaccaagactggggaaattgggggaaggctagagcgagatcatctggggacctggatagaccggagttcagctctcagccttgctgctcaagagtgaatgaccttgggtatattgcagagctgttgtgagtgtttttcttatgtaccttatgaggatgctataagaactaagtggcatgggtctatagccctcaaagatgacctggaagtagagtagatatttccatttctactggctaacctgggtcagaatggtaaccttttggatttattgttaaaccatgtacccttttctaggaggtgggaaagggacaaatgagggaagtgaggctgaaggagctgaagggatacttcactgcaaatgggtgtcagaggcaggtctaggatccaagaccgggactcctcctacagtgcttctgtggctacagcccaccgtcttggcatacgagccagggcgcactgggtgtgggtgttcccagccgtgcctccccggctctggggaccagcctgaccatgccctctcccccaggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccrgccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccagscaacttcagtcctgggagacccatgtaaataccagactgcaggttggaccccagagattcccaagscaaaaaccttagctccctcccrcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtagaaggcagcagtgccacttctgtgcttggcattcaagtataggaagacccctgtgtctgcagggtctaacccaagggaagccaggttgcccccactgntccacctcccctgttgcagctcctgcttcctctgaaggactcatcctttgccctcttacccaccagggcagagaaggctctgtggaaaaggtggccttggatgcactggcattgcctgtgtctgcctatgtcccttgncctgtcttctgtcccatgtcaggatccccttcctctagggcaggctgggagaagcagggaaggccctgaccactgcggcctggacagttctccctcctctcagcttccagggcggtcccaagctccaagccttccgggggaaaaacttggtactgccccaacaacagaaacttggctttctacaaatgaagcgtaaatcancccagtgagggaggaatattcttaccaccttgagaataaccgcagtgatgacaaacaaggtgccagcacccacgggctcaggcgctggggagctgtcagggcgtaatttgcatgctaaatacaatatttttagcaccaaagtttggagcacttaacttgccctgaacagttaattatggactttgatcttctccttaaacctaaaggtagcactaagccctgggagaggctcctgtccccaggagcaccctgattctggaaagtgagcaaaacaggcccctagtctaactcggactgggtcataacaccaaggacccagtgaccatctcctctggaaagcatcaggtccccaaggggtctagaagccccagggacccaacccatccccattgnacacataccatgctcaatgtctgtgaaagatcttggcctggatggacgcttaaaagtatatcccacaattaggaatcttatgagggtatacaggcttatcagatgtgangattggaagagatgacaaagagaagcagaggaaagaagaaggaagggagggagggagagagggagggacggaagggagacccagagcagagtgagaagagcattgacagggagcagaggggaagagggcagngcaggggcggnaggcggtgcaggggaaagttgcccacagttgtcacgaggcttcatgtctttctgccagacagcagattgacagctagagtgggcaggggagggctgggctccaccctctccccccctcagcacttcaggggcaaagaaatgggaggagtaggacccgacacgacacgggaacacaaggtggaagggggtggcccaggctctgactctctccagagaggtcctgacgatggtctcttgctcttgacgacaggatggaaaggaagcctccagttttcactcctctttgcctttccctaggtttctgtctgtgnctgtgccgctctgtagagtgccttttaatcaaaggatcattcatttgcctttacagcaggtagagtctgcacccttgtcccctgccctgccctttcctagagcacagcccaggatcaccacctaagggccaggcacagtggggcactttgcatatgtcgtcttagatgggaatggaaatcacacagtcacaaaggagcagacccagaggtaactgcaagtcttcaaggcttcaggccctcgttcttcctcctgggtcctgaaggactctcaggtggccctgggctggggaaagttcctgggaattagaagacgagttgtctagcagactaagaagttgcattgcttcgtccacccatctgctgtcttccttagggaatatttattgagcaattcttgtgctccaggccctgggtgaggtgctgggattaaaacagtcaacaatggtcaccggccctgccttcgtgagtccagtctagtgggatcacaacaaagtgagactctgcttcccccaaggcagagcccagggacggcagacacagcacaggcacatgctgcgcacttccaaggcacctttctcctagatgacgtgtgacacagcagccctggtgtgccttggccttgaacaattaggtagggcaccagtaggggcaggaagactgacagcatggaccactacttcctgcgtggctggggcaggaggccctgaaagaagcagctacagatcctgctttccaggtggtctcaccacaggcacagccagttgcctgcaactaagaacactgaagcccggcctgctttgggggcatttccagcatcccctcctatctggaatctcttcccaaaacatccctgtccccagggactgcatagctcctttcacctggtgggccagccctctctgtgtgccactggctccctgccntcctgggtcccaggacctgggctgagtgtgcaactttagcttccatctgcacagggccctctctgcgggctttgccctccctgatggcatgttcttctcctaacccactggagtgaggggcattcttatcgtctccacttacagaagagaagcctctcatacagaaagggtagtttccctttcaggctaaatcttctgattttacctcttatagtgtccattccagccctggcttctgaccagcgtgagtgcagctgaaagaggctaggatggtcagaagattttttttaaaagagaaagaatacttgttttggaccagacctgtagggtaacagcctggggtgcatgtaaacagaacttgggcctctccagggagcaagtgggtaggtggtgggggcagagcagttattagtggggcttcaggagtgagagggccagtctgggcatcggtgtgccagagggtcctctcctgccaaccaggtggacctggctctgctacaytagtttytgggggtgcaggggaagcaggaagctggtggggggagagtggaccatctttggagtagggagacctatcctggctttgtggggttagaggggacgtgggaagggggtacctggtaggaacaccttcctgcttcctctctcatcacagttctgctacttgscaaataaggcctcattggagcagaggcccagagaggcgcaggagagggaatggcaaggagggatgtggagagacctttcactcacagctggagccagagacaaccccaagttcataggaagtgctgtgtgaccgctccacttgcatgtggctttctcccactttcacatctggcttctgacacccagagctgcgtgtgaggccagggagagggggccacctccctggcaccccaggnccaggggtgtcagagctgcacaggaagtgaaggaatcctatcaggaaggggtgaggtggtcccattccaggaatgggagggcaggcctgggccaccgactgtgcagagacaaagctggccatccataccttctggattttcctgatcaggcccaactccaaacactctgtcctgttncccccaggagtgtgtaagttgggagggtcatttgggggccaggctctgtnctgcgtccagggcaggggaaagcatgggaggggcaagagaggcaggacactcccctggagaagacaagtcccttgttggttttctcccagggctgacagcacagacctgacccaggctctgggtagaggaggcatgactggctagtggggctagcctttgccctataactgacctgatccgtaggtgtttcccaggggncctcggtgtctcagcctacaccaaggcactgaaggagagtctccctttcctttcctgaatgtatcggcaaagaaaaatgggcagaggcttctaagagcatccttgcaaaaggctcccaagggatgggctgtctatgtcttattgcaagagaaacattcctctgatttaggaggaactgcaagaaatgaatgtgttgagtgcttattaactgccttgccctctgactggcagggcatcaggtttaattcttacaatgacccagagataaacattattgctgctagatgataccatcaagctaagatagttttagcaacctgagatctncaactanaaatggctcagcgaagacatgcacccaggtctgcctgcccgagtccactagacccagccacccgcagtttccctcnccgacctcagcccagcctggttcctgccctctgtccctaacattaccttacaccaaaaacccctgctgcctgtaatccgattcagcaagcagggaggaagcaccctctgagctccagacaccagggcactaagagaggactggatggaacacagaaacaagtagggcgtggtgatgtggcatgcaatgagcaaggaaggtggacccaagagaagctctgtcaacctcactgcactgggcatcgggacagaatgcccctgaggaaggagtgagcatctccagggggcaagcagagacgtcttcgtgggcaggaacaggcaagttggatctggaagggcagatcggatcccggcaggcaccggtggaagaagaggacacagcattagcaggaagaagggcctggggttggtgggtgctgcagcaatgctggctgggtttcagagtgggctnggagccctaactcactggaggccctcacttattggtgagatgtgttctataggtaagcacagggggnatcaggaaaggaatgttcctgaacacaagatcaccttggagctgycatctgtgtgttcaggaagtcctggggntccctccttcctctcctwcttgccacaggcctggagccctgtttcctccccttctcagacctgcccattatcattctcttcctggggctcagtgncctcaggttggctttggagagggacacggtctccccagtctcctcatttcccgtaggccttttcagccgngaggagttttctgctgatccctgcagtcctgcctcccttcaagacagtcgcggcagnctgtttaggcctctgccctacactttgntgtggagacagnttgmcaagnctccctgytcctttaggattcccagcctcataactggagggttccaccaacagcttccatctcccagcacaagaagggatccaggtggaacaatctcagctatctgcatctctggggatggctaggctaggggatgccatctcccaggacccaggaatgtcctccttgaggtgggctggattggatcaagcacagtttaggatgggaaaggaggctgtcatatgagtaaagggtaatgaaggggttacctgagtaacactggtgagaaaggacaggaacagaaagctggaagaggagagaagtgatggtgagtaccaatgtgtgctggagataggcgtgatctaattgcatccccaagataacaaatctgttataaatacagatgccgaaatctggaaagtctgggtaaagtccacagctagcaaaaggcagaattgggatgccaagccaggtgtgccttgctctgaagccccatagggcagggtccccatctcccaccactgtcccccatttcctcatcctgcacacagatgaagaggtctttaggatgcatggngtgttctgattgnatcctccttctgctgggaaactgacaggaggtcccaactgtccctcatcctgcatgggagntcccaaatcagaatgaacattaaggtctctccattgaatacgcacctcccacacctgcacctcaacagnactgtccctccttcagntgggcactctctgctccccatagcagntgtgtntntttcacattccccagccaatcctctccccttntatccagtccaggctttgctcctcttntgctctgtccttgtcctcattcaacatatgcatcttccaggaaaatctttcctgatcgaccccacgcaacatggcaactgcctggcggaggcaagacaggtatagactgtgctgtccactgtccccatgtggctagatacccacatttaatttattgaaataaaaaaacttgattccttggttacactagccacatttcaagtgctcaggagcatctgtggctggcggctatcctactgagcagtgtggatgtagaaggcttccatcctcccagtgctgatccagaatggctcctaagttaggagtttggataccttcaactaaaatagggtataaagggtgaagtcaggttctccctccaagtggagatgcccagtaagaaattggggagctctttccccccgatccccaatctcccctaaatagggcttgaacttctactggagaccctctccttgggaatttcaagggcctcattcaaacccaattttaaacatccctagggaagctctagaaggcagctgtgttgaaatgggactcaaggtggataccgtgtgtgtttgtgtgtgtgtctgtgtctggctaggagtggcttgatagggcagaagaagacactggtaggcaatgaagggccatgttgtccagggctggcctgaagtcacctctaagagttatctcttagaggaaggaagtggaggaagcaggggtgtgagaatctctgcctagactcaggatcaccagagctgaaatgattcccagggacagtcctgtccacatctccacagcatccctgccattcattggacactaggtgcaaaaggttcatctactttttctaactcttttcttcaaggccttttctcagttctcagctctttccaataggatcagccagccactcttctccctctttggttccctcctgatcactcccactgatggatctgagcacccttggctggaagtatgtagccccagagccttctctggctctcatagagtcctagatttggagttagatctggaagggcagataggatcctggcagacactggtgggagacttcagagtgcaaagagaagcaaacagccctgtgcctcctccacttccctcctcttgctcccgcatgttctgggaaagcttagaactcctaggggaatctttaactacaagttttgcctaaggcttagtctcagttgagactaaagagatttgagcctgggcagctagcaggrggagtagggaagatggaacagtcaggtggagtgtcccctggaaacccagttcgtcagggagtccaaatgggaagaggaaaaaggacttctggacaacccaagctgctgggccaactgatgtctgcaaggttatggctaaggaagggcacagagagaaaccatgggatgctccagtgcttctcagccctgggtgctcttcagaatcacttgggaattgttaatgatgctcacaccaaggtcctaccctggaggttctggtttaattggttgagggtagggccaggcgttggtatattttaaaagccccttgcccaggagattttaacgtgcaggctcggtttataaccatggctctaggacacactccagcttcctgggctcacatctgactttccatggtggccataagctatggcaaggtggtggtaaagggcccagggcccaacaggctggagctgtggtccagaggccaaaggatggaggacacaaagctgctagctctactgaaagggacttgttaattcaacaggattgcacgtgggcaacacattttcntctgccccacctctctcaccccatgctgatgccaggctgctagctgctgtccccatgcccagactggagtgcccagcccaggagaatgacacgcagaggtgggcctgccaatcagcttggctgagagctggccaatagttgggacagatgattccttttctctgacaattgcaaaacatccttgagtgggaggagctgcttctactatgctgaccagcagtgtagggagacagaaggagcaggataggaccccaggaaactcatcctgtccctactccctccaaattgttggctgctctcctggaagctgaactagggccctggtcacataaagttagtgtgaacagctccaagccacaaccagcaggacaggacaggacagctgatgggtggcctgggtgtggtcacaaaagtgagccctgcaagctccatactgagtccagacaacccacccctcaccatcatcaccccctccaactccctctgctgcctctgcctcctgtctttatgtgaactcacacacacatatgaacacacatataccctacattagacaaatcgcatacacacacaagcacaccatactaacgctgctggcctctatatgcaacttggttccacaggccaatccattctctaaacaggagcctcaggagcctcagctttcttgacttcaggagtttgaaatctggctgtaaaactggacacgatgtaggatgaagggtggtgctggggaccagggacctcacatctcacatagggtgacttatccacagttggagtgcacccttcctatatcctcaaaatgccatgagggagacccaggcattgggaccaggggcactggattaggtgtggtcagtgtggcaacagatggctggctgggactttagtgagcagaaacctcctttgaacttttagcttctgccccttgagcaaagcacatgtgtctggctctccccgccccagcctgaggtaaggagccaagagcccaagacgtgagatgggaggatcctgcttccctcaccgctgtgcctccctccatctccagagggggtctgctaggatgcaagggcccctgtgaactcagaagcgagtggcaaaaggtggtaggtgtcttggattggagagggtattcatttggggagcttgcatccccataaaggaggaggaggaggcctggggtctgttggaagtgactggaggatgtgttgaaggcagctctccaagccagagccccttcctcagctacagccacagtgatacctctccccatgcccccatcacccaggttcctgtctcctctcaaggtggaaggggcaggtgaagactgcagacagggaagatgccctgccagaagcccagctgcactttcaccaattcntactccatccaggcggagaggccccaagtagtctaaatttctttctttctttctttttnatatggagtctcgctctgttgcccaggctggagtgcagtggtgcgatctcggctcactgcaacctctgcctcctgggttcaaggaattctcctgcctcagcctccctggtagttgggattacaggcacgtgccaccatacccagctaaattttgtatttttagcagagacagggttttgccatgttggccaggctggcctcaaactcttgatctcaggtgatctgcctgcctcagcctcccaaagtgctgggattacagacgtgagccaccacggctggccaagttgtctaaatttccatctcggctcctggcttagaaccacccagagtggccactgacggctccttgccctctaggaaggacatgatgccctgctttcggctgcggagggccagttgcaggggtgtgcagctcactccatcctggacgtccagctgggcgcctgcctcgaccagcactttgaggatggctgtgttgcccttgagggcggccaggtgggcgggtgtccagcccaccttgttgcgggcgtggacatttgcgtgatgttctakgaggttgatgacactcaggaaggtgctcctctggaccgccaggtggaggggtgtccagcctgactgctctgcagcattggggtcagccccacactgcagcagtgctgacaccaccgcctcctccccgtggcgtgcagctaggtgcaggggagtccagttcacagctccaagagcacccatgtttgcgtggctctctgccagcagatggatgatctccargtggcccttgtangctgctagatgcaggggtgtccaaccctggtgggtgggcagctcaagctggctccgtacctgagcacatcttgcagatcaggtatttgcccctggcagctgcagtgtgcagtgggccatagccgctctggtcaagggcatcagggaccgctccactcttcagcaggtgttggatggccctcactttgccccgctctactgccaggtgcagtggtgttctcaggtttctctgctgancatccaactcagccccctggctggtcagcatcttgaccaggctaacatggccaaagtaggcggccacatggaggggggtcttgccctcagcctcacgcaggttggggtcagcctgacgggagaccagaagccgtgccacattctcaaagttattctgtgcagccagntgaagangggtccacccttcacgttcctgggcatccacacaggccccgtggtccaagaacangcgcgcantgcggtcatccccattctgggctgcaaantgcantggggcc

SEQ ID NO: 15 - protein, 5 aa, Artificial sequence
  Synthetic peptide
GRGDS

SEQ ID NO: 16 - protein, 6 aa, Artificial sequence
  Synthetic peptide
GRGESP

SEQ ID NO: 17 - DNA, 26 bp, Artificial sequence
  Synthetic single strand DNA oligonuclotide
ggaattccaggtttctgtcagatgcc

SEQ ID NO: 18 - DNA, 26 bp, Artificial sequence
  Synthetic single strand DNA oligonuclotide
ggaattccgttgctgagttttcgaac

(19) United States
(12) Patent Application Publication
(10) Pub. No.: US 2005/0163773 A1
(43) Pub. Date: Jul. 28, 2005

(54) Methods and pharmaceutical compositions for dopaminergic modulation of t-cell adhesion and activity

(76) Inventor: Mia Levite, Savyon (IL)

Correspondence Address: G E Ehrlich;Anthony Castorina, Suite 207, 2001 Jefferson Davis Highway, Arlington, VA, 22202, US

(21) Appl. No.: 10/489,861

(22) PCT Filed: Oct. 29, 2002
(86) PCT No.: PCT/IL02/00870

Related U.S. Application Data
(60) Provisional application No. 60/330,692, filed on Oct. 29, 2001.

(51) Int. Cl. A61K 39/395; A61K 48/00; C12N 15/85

(52) U.S. Cl. 424/143.1; 514/44; 435/455

(57) ABSTRACT

Methods and materials comprising Dopamine, Dopamine analogs, polynucleotide constructs and anti-Dopamine receptor antibodies for immune enhancement and suppression, prevention and treatment of diseases and conditions characterized by abnormal T-cell activity, and treatment of T-cell related neoplastic diseases are disclosed.

### FIELD AND BACKGROUND OF THE INVENTION

The present invention relates to methods and compositions for the modulation of T-cell activity by Dopamine and specific Dopaminergic receptor functional analogs.

T-Cells in Immunity and Disease

Immune responses are largely mediated by a diverse collection of peripheral blood cells termed leukocytes. The leukocytes include lymphocytes, granulocytes and monocytes. Granulocytes are further subdivided into neutrophils, eosinophils and basophils. Lymphocytes are further subdivided into T and B lymphocytes. T-lymphocytes originate from lymphocytic-committed stem cells of the embryo. Differentiation occurs in the thymus and proceeds through prothymocyte, cortical thymocyte and medullary thymocyte intermediate stages, to produce various types of mature Ap T-cells. These subtypes include CD4+ T cells (also known as T helper and T inducer cells), which, when activated, have the capacity to stimulate other immune system cell types. The T-helper cells are further subdivided into the Th1, Th2 and Th3 cells, primarily according to their specific cytokine secretion profile and function. T cells also include suppressor regulator T cells (previously known as cytotoxic/suppressor T cells), which, when activated, have the capacity to lyse target cells and suppress CD4+ mediated effects.

T-cell activation: Immune system responses, are elicited in a variety of situations. The most frequent response is as a desirable protection against infectious microorganisms. The current dogma is that in the organism, under physiological conditions, resting T-cells are activated and triggered to function primarily by antigens which bind to T-cell receptor (TCR) after being processed and presented by antigen- presenting cells, or by immunocyte-secreted factors such as chemokines and cytokines, operating through their own receptors. Experimentally, T-cells can be activated by various non-physiological agents such as phorbol esters, mitogens, ionomycin, and anti-CD3 antibodies. To identify novel physiological means directly activating and/or regulating T-cells in conditions of health and disease, especially in non-lymphoid environments (e.g. brain) and in a TCR-independent manner, remains a challenge of scientific and clinical importance.

In recent years, it has become evident that specific immune responses and diseases are associated with T-helper (Th) functions. Among these are anti-viral, anti-bacterial and anti-parasite immune responses, mucosal immune responses and systemic unresponsiveness (mucosally induced tolerance), autoimmune reactions and diseases, allergic responses, allograft rejection, graft-versus host disease and others. Furthermore, specific T-cell mediated proinflammatory functions may have either beneficial or detrimental effects on specific neoplasias: on the one hand, proinflammatory cytokines may assist in anti-tumor immune surveillance, and, on the other, elevated levels of proinflammatory cytokines were found within chronically inflamed tissues that show increased incidence of neoplasia.

In general, CD4+ T-cells can be divided into at least two major mutually exclusive subsets, Th1 and Th2, distinguished according to their cytokine secretion profile. Th1 cells secrete mainly INF-γ, TNF-β and IL-2, their principal effector function being in phagocyte-mediated defense against infections. The Th1 cells are usually associated with inflammation, and induce cell-mediated responses.

Essential and beneficial immunity cannot take place without Th1 cytokines, but their over or dis-regulated production leads to numerous detrimental clinical consequences. Th2 cells induce B-cell proliferation and differentiation, and thus, induce immunoglobulin production. Cytokines from Th2 cells (mainly IL-4, IL-10 and IL-13) can also antagonize the effects of Th1 cell-mediated reactivities, inhibiting potentially injurious Th1 responses.

Clinical application of cytokine effects is widespread and well documented, particularly for the proinflammatory TNF-α and the immune-suppressory IL-10. Treatment with IL-10 has been proposed for management and prevention of such diverse inflammatory disorders as Type I diabetes, multiple myeloma, LPS-induced septic shock (U.S. Pat. No. 6,410,008 to Strom, et al); preterm labor associated with inflammation (U.S. Pat. No. 6,403,078 to Fortunato and Menon) and IL-2 dependent neoplastic tumors and conditions (U.S. Pat. No. 6,319,493 to Vieira et al). Furthermore, effective clinical protocols have been developed for IL-10 treatment of psoriasis, B-1 malignant disease, allograft rejection and Crohn's disease (see www.laboratory.gg/IL-10 workshop).

Treatment of inflammatory conditions such as Rheumatoid Arthritis, Ankylosing Spondylitis and Crohn's disease, as well as other non-inflammatory conditions, with anti-TNF-α drugs is widespread in the U.S. (see, for example, U.S. Pat. Nos. 6,219,899; 6,291,487; 6,294,350 and 6,221,851 to Schwartz, Chihiro et al, Peterson, and Feldman, respectively). However, these anti-TNF-α therapies (etanercept, infliximab, etc) have been shown to cause dangerous neurotoxicity and demyelination-like illness.

T-cell migration: and integrin-fibronectin binding: Adhesion is important for a cell: it provides anchorage, traction for migration, signals for homing and regulates growth and differentiation. In the immune system, the ongoing migration, extravasation and homing of T-cells from the blood stream to various tissues and organs is crucially dependent on various adhesive interactions with ligands on target cell-surfaces and matrices.

A class of glycoproteins has been identified as comprising the receptors in the cell recognition system for cell-extracellular matrix interaction. These proteins, referred to as integrins, are characterized by the involvement of the RGD sequence in ligand recognition, and appear to play a significant role in the assembly of the extracellular matrix (Ruoslahti, E. “Versatile Mechanisms of Cell Adhesion,” The Harvey Lectures, Series 84, pp 1-17 (1990)).

An integrin molecule is a heterodimeric membrane protein composed of one α and one β subunit. Several subunits of each kind are known, and various combinations of these subunits make up receptors with differing ligand specificities. The ligands for integrin are extracellular matrix proteins such fibronectin, lamanin, collagens and vitronectin or membrane proteins at the surface of other cells. By binding to their ligands, integrins mediate the adhesion of cells to extracellular matrices and to other cells.

Integrin functions have been shown to play a key role in a broad spectrum of normal and diseased conditions in general, and in inflammation and injury in particular. For example, T-cell recruitment into inflamed gingival tissues in periodontal disease (Taubman and Kawai, Crit. Rev Oral Biol Med 2001, 12(2) 125-35), and into the lamina propria in intestinal inflammation is associated with increased integrin expression. Normal cells are anchorage (integrin-fibronectin) dependent for progression through the cell cycle, is whereas cancer cells exhibit anchorage-independent mitogenic activity. Furthermore, since resting T-cells cannot adhere, integrin-mediated fibronectin binding is indicative of significant activation and induction of T-cell function.

Three major events are involved in inflammation: (1) increased blood supply to the injured or infected area; (2) increased capillary permeability enabled by retraction of endothelial cells; and (3) migration of leukocytes out of the capillaries and into the surrounding tissue (Roitt et al., Immunology, Grower Medical Publishing, New York, 1989). Increased capillary permeability allows larger molecules to cross the endothelium that are not ordinarily capable of doing so, thereby allowing soluble mediators of immunity such as leukocytes to reach the injured or infected site. Leukocytes, primarily neutrophil polymorphs (also known as polymorphonuclear leukocytes, neutrophils or PMNS) and macrophages, migrate to the injured site by a process known as chemotaxis. At the site of inflammation, tissue damage and complement activation cause the release of chemotactic peptides such as C5a. Complement activation products are also responsible for causing degranulation of phagocytic cells, mast cells and basophils, smooth muscle contraction and increases in vascular permeability (Mulligan et al. 1991 J. Immunol. 148:1479-1485).

The traversing of leukocytes from the bloodstream to extravascular sites of inflammation or immune reaction involves a complex but coordinated series of events. At the extravascular site of infection or tissue injury, signals are generated such as bacterial endotoxins, activated complement fragments or proinflammatory cytokines such as interleukin 1 (IL-1), interleukin 6 (IL-6), and tumor necrosis factor (TNF) which activate leukocytes and/or endothelial cells and cause one or both of these cell types to become adhesive. Initially, cells become transiently adhesive (manifested by rolling) and later, such cells become firmly adhesive (manifested by sticking). Adherent leukocytes travel across the endothelial cell surface, diapedese between endothelial cells and migrate through the subendothelial matrix to the site of inflammation or immune reaction (Harlan et al., Adhesion-Its role in Inflammatory Disease, W. H. Freeman & Co., New York, 1992).

Although leukocyte traversal of vessel walls to extravascular tissue is necessary for host defense against foreign antigens and organisms, leukocyte-endothelial interactions often have deleterious consequences for the host. For example, during the process of adherence and transendothelial migration, leukocytes release oxidants, proteases and cytokines that directly damage endothelium or cause endothelial dysfunction. Once at the extravascular site, emigrated leukocytes further contribute to tissue damage by releasing a variety of inflammatory mediators. Moreover, single leukocytes sticking within the capillary lumen or aggregation of leukocytes within larger vessels are responsible for microvascular occlusion and ischemia. Leukocyte-mediated vascular and tissue injury has been implicated in pathogenesis of a wide variety of clinical disorders such as acute and chronic allograft rejection, vasculitis, rheumatoid and other forms of inflammatory based arthritis, inflammatory skin diseases, adult respiratory distress syndrome, ischemia-reperfusion syndromes such as myocardial infarction, shock, stroke, organ transplantation, crush injury and limb replantation.

Many other serious clinical conditions involve underlying inflammatory processes in humans. For example, multiple sclerosis (MS) is an inflammatory disease of the central nervous system. In MS, circulating leukocytes infiltrate inflamed brain endothelium and damage myelin, with resultant impaired nerve conduction and paralysis (Yednock et al., 1992 Nature 366:63-66). Systemic lupus erythematosus (SLE) is an autoimmune disease characterized by the presence of tissue damage caused by self antigen directed antibodies. Auto-antibodies bound to antigens in various organs lead to complement-mediated and inflammatory cell mediated tissue damage (Theofilopoubs, A. N. 1992 Encyclopedia of Immunology, pp. 1414-1417).

Reperfusion injury is another condition associated with activation of the inflammatory system and enhanced leukocyte-endothelial cell (EC) adhesion. There is much evidence that adhesion-promoting molecules facilitate interactions between leukocytes and endothelial cells and play important roles in acute inflammatory reaction and accompanying tissue injury. For example, in acute lung injury caused by deposition of IgG immune complexes or after bolus i.v. infusion of cobra venom factor (CVF), neutrophil activation and the generation of toxic oxygen metabolites cause acute injury (Mulligan et al., 1992 J. Immunol. 150(6):2401-2405). Neutrophils (PMNs) are also known to mediate ischemia/reperfusion injury in skeletal and cardiac muscle, kidney and other tissues (Pemberton et al., 1993 J. Immunol. 150:5104-5113). Infiltration of airways by inflammatory cells, particularly eosinophils, neutrophils and T lymphocytes are characteristic features of atopic or allergic asthma (Cotran et al., Pathological Basis of Disease, W. B. Saunders, Philadelphia, 1994). Cellular infiltration of the pancreas with resultant destruction of islet beta-cells is the underlying pathogenesis associated with insulin-dependent diabetes mellitus (Burkly et al. 1994 Diabetes 43: 529-534).

Activation of inflammatory cells whose products cause tissue injury underlies the pathology of inflammatory bowel diseases such as Crohn's disease and ulcerative colitis. Neutrophils, eosinophils, mast cells, lymphocytes and macrophages contribute to the inflammatory response. Minute microabcesses of neutrophils in the upper epithelial layers of the dermis accompany the characteristic epidermal hyperplasia/thickening and scaling in psonasis.

Various anti-inflammatory drugs are currently available for use in treating conditions involving underlying inflammatory processes. Their effectiveness however, is widely variable and there remains a significant clinical unmet need. This is especially true in the aforementioned diseases where available therapy is either of limited effectiveness or is accompanied by unwanted side effect profiles. Moreover, few clinical agents are available which directly inhibit cellular infiltration, a major underlying cause of tissue damage associated with inflammation. Thus, there is a need for a safe, effective clinical agent for preventing and ameliorating cellular infiltration and consequential pathologic conditions associated with inflammatory diseases and injuries.

Modification of T-cell activity: Therapeutic application of T-cell modulating agents has been proposed for the treatment of conditions characterized by both immune deficiency and chronic inflammation. For example, U.S. Pat. No. 5,632,983 to Hadden discloses a composition consisting of peptides of thymus extract, and natural cytokines, for stimulation of cell mediated immunity in immune deficient conditions. Although significant enhancement of a number of cell mediated immune functions was demonstrated the effects were highly non-specific, as could be expected when employing poorly defined biologically derived materials.

Recently, Butcher et al. (U.S. Pat. No. 6,245,332) demonstrated the specific interaction of chemokine ligands TARC and MDC with the CCR4 receptors of memory T-cells, enhancing interaction of these cells with vascular epithelium and promoting T-cell extravasation. Therapeutic application of CCR4 agonists was disclosed for enhanced T-cell localization, and of antagonists for inhibition of immune reactivity, as an anti-inflammatory agent. Although the ligands were characterized, and identified in inflamed tissue, no actual therapeutic effects of agonists or antagonists were demonstrated.

Inhibition of a number of T-cell functions has been the target of many proposed anti-inflammatory therapies. Haynes et al. (U.S. Pat. No. 5,863,540) disclosed the use of anti-CD44 (cell adhesion molecule effecting T-cell activation) antibody for treatment of autoimmune conditions such as Rheumatoid Arthritis. Godfrey et al. (U.S. Pat. No. 6,277,962) disclosed a purified ACT-4 T-cell surface receptor expressed in activated CD4+ and CD8+ T-cells, and proposed the use of anti-ACT-4 antibodies to achieve downregulation of T-cell activation. Similarly, Weiner et al. (U.S. Pat. Nos. 6,077,509 and 6,036,457) proposed treatment with peptides containing immunodominant epitopes of myelin basic protein (associated with Multiple Sclerosis) for the specific suppression of CD4+T-cell activity in this central nervous system autoimmune condition. However, none of the proposed applications were able to demonstrate any specific effect on the processes regulating expression of T-cell specific surface proteins responsible for immune activity.

Autoimmune Diseases

Autoimmune diseases are characterized by the development of an immune reaction to self components. Normally, tissues of the body are protected from attack by the immune system; in autoimmune diseases there is a breakdown of the self-protection mechanisms and an immune response directed to various components of the body ensues. Autoimmune diseases are for the most part chronic and require life long therapy. The number of recognized autoimmune diseases is large and consists of a continuum ranging from diseases affecting a single organ system to those affecting several organ systems. With increased understanding of the molecular basis of disease processes, many more diseases will likely be found to have an autoimmune component. Autoimmune diseases are typically divided into Organ Specific, and Non-Organ Specific Autoimmune disease. Specific examples of Organ Specific Autoimmune diseases are: Hashimoto's thyroiditis, Graves' disease, Addison's disease, Juvenile diabetes (Type I), Myasthenia gravis, pemphigus vulgaris, sympathetic opthalmia, Multiple Sclerosis, autoimmunehemolytic anemia, active chronic hepatitis, and Rheumatoid arthritis.

Rheumatoid arthritis is a systemic, chronic, inflammatory disease that affects principally the joints and sometimes many other organs and tissues throughout the body, characterized by a nonsuppurative proliferative synovitis, which in time leads to the destruction of articular cartilage and progressive disabling arthritis. The disease is caused by persistent and self-perpetuating inflammation resulting from immunologic processes taking place in the joints. Both humoral and T-cell mediated immune responses are involved in the pathogenesis of rheumatoid arthritis.

The key event in the pathogenesis of the arthritis is the formation of antibodies directed against other self antibodies. T cells may also be involved in the pathogenesis of rheumatoid arthritis. A large number of T cells are found in the synovial membrane, outnumbering B cells and plasma cells. Additionally, procedures to decrease the population of T cells (such as draining the thoracic duct) result in remission of symptoms.

Rheumatoid arthritis is a very common disease and is variously reported (depending on diagnostic criteria) to affect 0.5 to 3.8% of women and 0.1 to 1.3% of men in the United States.

Multiple sclerosis is a neurogenic disease that is thought to be caused by autoimmune mechanisms. The systemic immune response and the response of the central nervous system become involved. Although the cause and pathogenesis of multiple sclerosis are unknown, it is widely believed that immune abnormalities are somehow related to the disease. Suppression or modulation of the immune responses may be the key. Multiple sclerosis is modeled, in rodents, by the passive transfer of immune reactivity to Myelin Basic Protein via administration of sensitized T-cell (experimental autoimmune encephalomyelitis: EAE)

Myasthenia gravis is another nervous system related autoimmune disorder caused by antibodies directed against the acetylcholine receptor of skeletal muscle. In both experimental allergic myasthenia gravis and human myasthenia gravis, the extent of acetylcholine receptor loss parallels the clinical severity of the disease, suggesting that acetylcholine receptor antibody-induced acceleration of acetylcholine receptor degradation is important in the development of myasthenia gravis.

Other disorders, especially those presumed to be autoimmune in origin, can occur in association with myasthenia gravis. Thyroid disease, rheumatoid arthritis, systemic lupus erythematosus, and pernicious anemia all occur more commonly with myasthenia gravis than would be expected by chance.

One example of a non-organ specific Autoimmune disease is Systemic lupus erythematosus.

Acute attacks of Systemic lupus erythematosus are usually treated by adrenocortical steroids or immunosuppressive drugs. These drugs often control the acute manifestations. With cessation of therapy the disease usually reexacerbates. The prognosis has improved in the recent past; approximately 70 to 80% of patients are alive 5 years after the onset of illness and 60% at 10 years. Lifelong therapy is required to control the disease.

The foundation of therapy of autoimmune diseases is treatment with immunosuppressive agents. The basis for this therapy is attenuation of the self-directed immune response with the primary aim being to control symptoms of the particular disease. The drugs utilized to achieve this aim are far from satisfactory, in that adverse side effects are numerous and control of the disease is many times difficult to achieve. The problem is compounded by the chronicity of the disease with effective therapy becoming more difficult with time. An indication of the severity of particular diseases is seen in the willingness to accept greater risks associated with therapy as the disease progresses. Currently available therapy is distinctly non-selective in nature, having broad effects on both the humoral and cell mediated arms of the immune system. This lack of specificity can limit the effectiveness of certain therapeutic regimens. The main groups of chemical immunosuppressives are alkylating agents, antimetabolites, corticosteroids, and antibiotics, each will be discussed briefly.

The corticosteroids, also called adrenocorticosteroids, are fat-like compounds produced by the outer layer or cortex, of the adrenal gland. Therapeutic use of the corticosteroids for autoimmune disease is based on their two primary effects on the immune system, anti-inflammatory action and destruction of susceptible lymphocytes. They also effect a redistribution of lymphocytes from peripheral blood back to the bone marrow. The use of corticosteroids is not without adverse side effects however, particularly during the course of life-long treatment which is required for many of the autoimmune diseases.

Major side effects of steroids are: Cushing syndrome, muscle atrophy, osteoporosis, steroid induced diabetes, atrophy of the adrenal glands, interference with growth, susceptibility to infections, aseptic bone necrosis, cataract development, gastric ulcer, steroid psychosis, skin alterations and nervous state accompanied by insomnia.

Attempts to minimize side effects incorporate alternate day or less frequent dosage regimens.

Another recently developed immunosuppressive agent is the antibiotic cyclosporin A. The antibiotic has greatest activity against T cells and does not seem to have much direct effect on B cells. The drug is being evaluated for the treatment of autoimmune diseases for which it shows some promise. Side effects include hair growth, mild water retention, renal toxicity, and, in older patients, nervous system disorders symptoms have been observed.

Other drugs are used alone or in combination with those listed above and include gold salts and antimalarials, such as chloroquine. Another class of drugs, the non-steroidal anti-inflammatory drugs are used extensively in arthritis. These drugs provide analgesia at low doses and are anti-inflammatory after repeated administration of high doses. Nonsteroidal anti-inflammatory drugs all act rapidly and their clinical effects decline promptly after cessation of therapy. However, they do not prevent the progression of rheumatoid arthritis, do not induce remissions, and are frequently associated with dangerous gastrointestinal side effects. Immunostimulants, such as levamisol have also been used in many autoimmune diseases but side effects have generally limited their use. Clearly, new therapies and drugs for the treatment of autoimmune disorders are needed.

Neurotransmitters and Immune System Function

It is generally accepted that the immune, nervous and endocrine systems are functionally interconnected. The significance of direct neuronal signaling on immune system components, including T-cells, can be appreciated considering the extensive innervation of all primary and secondary lymphoid tissue; the presence of both peptidergic and non-peptidergic neurotransmitters in capillaries and at sites of inflammation, injury or infection; and the demonstrated expression of specific receptors for various neurotransmitters on T- cell (and other immune system components) surface membrane.

Specific modulation of immune function has been demonstrated for a number of neurotransmitters. Recently, neuropeptides somatostatin (SOM), calcitonin gene related peptide (cGRP), neuropeptide Y (NPY) and also Dopamine were found to interact directly with specific receptors on the T-cell surface, while substance P (Sub P) indirectly affected T-cell function. These neurotransmitters exert both inhibitory and stimulatory influence on T-cell cytokine secretion, adhesion and apoptosis, depending on T-cell lineage and activation states (Levite, M.: Nerve Driven Immunity. The direct effects of neurotransmitters on T-cell function. Ann NY Acad Sci. 2001 917: 307-21). Similarly, physiological concentrations of the neurotransmitters SOM, Sub P, cGRP and NPY were found to directly induce both typical and non-typical cytokine and chemokine secretion from T-cells and intestinal epithelium, thus either blocking or evoking immune function (Levite, M. Nervous immunity: neurotransmitters, extracellular K⁺ and T-cell function. Trends Immunol. January 2001;22(1):2-5). Clearly, immune function is sensitive to neurogenic control.

A number of therapeutic applications of immune modulation by manipulation of neurotransmitters have been proposed. In one, botulinum toxin's peptide-lytic activity is employed to reduce the effect of immune-active neurotransmitters Sub P, cGRP, NK-1, VIP, IL-1 and IL-6 and others on neurogenic inflammatory conditions such as arthritis, synovitis, migraine and asthma (U.S. Pat. No. 6,063,763 to First). Hitzig (U.S. Pat. No. 5,658,955) proposes the combined application of neurotransmitters Dopamine and serotonin for complex inhibition and stimulation of various immune functions, for the treatment of AIDS and HIV infection, cancers, migraine, autoimmune inflammatory and allergic conditions, chronic fatigue syndrome and fibromyalgia. On the whole, however, the immune modulation of these inventions is of a broad and non-specific nature, with significant likelihood of undesirable complications and side effects in practice. In addition, no clear mechanism of action was defined for the immune-modulatory effects of Dopamine and serotonin in the latter disclosure. Thus, there is a need for improved methods of modulation of immune function via specific neurotransmitters and defined pathways of immune activation.

Dopamine: Dopamine, a catecholamine derived from tyrosine, is one of the principal neurotransmitters in the central nervous system, and its neuronal pathways are involved in several key functions such as behavior, control of movement, endocrine regulation and cardiovascular function Dopamine has at least five G-protein coupled receptor subtypes, D1-D5, each arising from a different gene. Traditionally, these receptors have been classified into D1-like (the D1 and D5), and D2-like (the D2, D3 and D4) receptor subtypes, primarily according to their ability to stimulate (or inhibit) adenylate cyclase (respectively), and to their pharmacological characteristics Receptors for Dopamine (particularly of the D2 subclass) represent the primary therapeutic target in a number of neuropathological disorders, including schizophrenia, Parkinson's disease and Huntington Chorea.

Can Dopamine, by direct interaction with its receptors, affect the function of the immune system in general, and that of T-cells in particular? Lymphocytes are most probably exposed to Dopamine since the primary and secondary lymphoid organs of various mammals are markedly innervated, and contain nerve fibers which stain for tyrosine hydroxylase (TH) [9, 10], the enzyme responsible for Dopamine synthesis. Furthermore, catecholamines and their metabolites have been demonstrated in single lymphocytes and extracts of T- and B-cell clones, and pharmacological inhibition of tyrosine hydroxylase reduces catecholamine levels, suggesting catecholamine synthesis by lymphocytes. However, neither Dopamine alone, nor Dopaminergic agonists, were ever shown to activate T-cell function by themselves.

The existence of putative Dopamine receptors of the D2, D3, D4 and D5 subtypes on immune cells, mainly on the heterogeneous population of human peripheral blood lymphocytes (PBLs), has been proposed by several authors, primarily on the basis of Dopaminergic ligand binding assays and specific mRNA expression, as monitored by RT-PCR. In addition, in vivo and in vitro experiments using Dopaminergic receptor agonists, such as bromocriptine, and antagonists, such as spiperone, haloperidol provide evidence of a role for Dopamine in modulating, primarily suppressing, immune function.

Nevertheless, it remains unclear which Dopamine receptor subtypes are actually expressed on T-cells, whether they are functional, and whether Dopamine, via such receptors, could directly activate T-cells.

Dopamine, Dopamine receptors and neurogenic disease: Receptors for Dopamine (particularly of the D2 class) represent the primary therapeutic target in a number of neuropathological disorders, including schizophrenia, Parkinson's disease and Huntington Chorea (see Seeman, P., Dopamine Receptors in Human Brain Disease. In Creese,I. and Fraser, C. M. (ed), Dopamine Receptors. Alan R. Liss 1987, p. 233-245). Recent studies have demonstrated increased levels of RNA encoding the D3 Dopamine receptors in peripheral blood lymphocytes (PBLs) of schizophrenic patients. Interestingly, these changes occurring in the immune system are the converse of those occurring in the nervous system, since a selective loss of Dopamine D3-type receptor mRNA expression was observed in parietal and motor cortices of patients with chronic schizophrenia.

Changes in the Dopamine receptors mRNA in lymphocytes were also observed in Parkinson's disease: PBLs of Parkinson's patients were shown to express reduced levels of the Dopamine D3 receptor mRNA, as compared to age-matched controls, and elevated levels of the D1-like and D2-like Dopamine putative receptors. Interestingly, a reduced density of Dopamine D2-like receptors was reported in PBLs of Alzheimer's patients, consistent with the observation of changes in the expression of D2-like receptors in Dopaminergic brain areas in Alzheimer disease patients.

Finally, elevated levels of putative D3, D4 and D5 Dopamine receptors were found on peripheral blood lymphocytes of migraine patients, potentially reflecting the Dopaminergic hypersensitivity displayed by migraineurs. Thus, it has been suggested that the altered expression of specific Dopamine receptor subtypes in the total cell population of peripheral blood lymphocytes, could be used as a peripheral marker for clinical diagnosis of the respective diseases.

It is possible, however, that the Dopamine receptors on PBLs in general, and on T-cells in particular, are not ‘passive’ markers for various diseases, but rather functional entities which upon direct stimulation by Dopamine, trigger T-cell function. If so, Dopamine-mediated T-cell function may be either up- or down-regulated in the different diseases, and as such, would play an important role in the ensuing pathological scenario.

Neuroprotective Immunity: In the context of neuroimmune interaction, and Dopamine's effects in the CNS, the recent discovery of neuroprotective interactions between T-cells and neuronal tissue in neurotoxicity, disease and injury is intriguing. Several studies by Schwartz, et al have shown that T-cell deficient mice are more susceptible to experimentally induced neuronal injury and neurotoxicity, and that reconstitution with wild-type splenocytes can effectively restore resistance. Additional evidence for such protective autoimmunity in CNS trauma was provided by the demonstration of potentiation of neuronal survival by prior, unrelated CNS insult in autoimmune encephalomyelitis-resistant strains of mice (see, for example, Yoles, et al, J Neurosci, Jun. 1, 2001;21(11): 3740-48; Kipnis, et al, J Neurosci Jul. 1, 20001;21(13):4564-71; and Schori, et al, J Neuroimmunol Oct. 1, 2001:119(2):199-204). Clinical application of such neuroprotective immunity has been proposed, employing vaccination with non-pathogenic CNS derived peptides such as MBP to boost innate beneficial autoimmunity (Schwartz and Kipnis, Trends Mol Med June 2001;7(6):252-58; and Schwartz, Surv Ophthalmol May 2001;45 Suppl 3:S256-60) and stimulation of peripheral monocytes for enhancement of axonal regeneration (U.S. Pat. No. 6,117,242 to Eisenbach-Schwartz). No mention is made of Dopamine or Dopamine analog modulation of T-cell activity, and furthermore, the authors note the substantial risk of inducing undesired autoimmune disease using immunization with self antigens.

Studies of lymphocyte activation in other neurogenic conditions also indicate a potential neuroprotective role of immune cells: in patients with encephalitis and MS, the beneficial brain-derived-neurotrophic-factor BNDF is secreted by immune cells in response to CNS auto-antigen stimulation (Kerschensteiner, et al, J Exp Med Mar. 1 1999;189(5):865-70). Furthermore, in clinical trials of an altered peptide ligand of myelin basic protein administered to patients with relapsing-remitting MS, reduction in lesion volume and number was achieved in the MBP-treated patients compared to the placebo group. However, the dosage required was high (5 mg), and the trial was suspended due to undesirable side effects (hypersensitivity). No mention was made of Dopamine stimulation of T-cells.

Neuroimmunology and Psychopathology: Many studies have demonstrated significant correlation between immune function and a variety of emotional and psychopathological conditions, especially schizophrenia and suicide (see, for example, Sperner-Unterweger B, et al, Scizophr Res 1999; 38:61-70; Staurenghi A H, et al Psychoneuroendocrinology 1997;22:575-90; van Gent T, et al J Child Psychol Psychiatry 1997;38:337-49; Nassberger L and Traskman-Bendz L Acta Psychiatr Scand 1993;88:48-52; and Dabkowska M and Rybakowski J Psychiatr Pol 1994;28:23-32). Presently it remains unclear whether the dysfunctional immune responses observed contribute to the psychopathogenic processes, are secondary to them, or a combination of the two.

T-cell enhancement has been observed in schizophrenia, and has been suggested as a marker of therapeutic outcome or neuroleptic treatment (Muller, et al Acta Psychiatr Scand 1993;87:66-71and Sperner-Unterweger B et al Scizophr Res 1999;38:61-70). The authors made no mention of T-cell-related therapy or Dopamine modulation of T-cell activity for treatment or prevention of the abovementioned disorders.

Manipulation of immune cells for therapy of brain related disorders has been proposed by Wank (Intern Pats. WO9950393A2 and WO9950393A3 to Wank, R). Wank describes the in-vitro activation of peripheral blood monocytes (PBMC), or phagocytes, for the treatment of a variety of brain-related disorders, including psychoses, schizophrenia, autism, Down's syndrome, disturbances of cerebral development and brain injury, based on the observation of inadequate immune responses in these conditions. In a report documenting adoptive immunotherapy of patients suffering from bipolar disorder, schizophrenia or autism, Wank describes the in-vitro activation, and reintroduction of the patients' own T-cells, in order to combat “chronically infected”, understimulated lymphocytes thought associated with these disorders. In this form of therapy, the T-cells are not stimulated directly, rather via monoclonal antibodies against the CD3 polypeptide complex, and IL-2. The patients were required to endure numerous weekly treatments (up to 104 weeks in one patient), and although improvement in some symptoms was noted, additional therapies were continued during and after these trials of adoptive immunotherapy. No mention is made of direct stimulation of T-cells with neurotransmitters, of specific T-cell response to therapy, or of treatment with Dopamine or Dopamine analogs.

To date, the dynamics of Dopamine interaction with specific Dopaminergic receptors on normal and diseased human T-cells have not been addressed directly.

While reducing the present invention to practice, the present inventor has uncovered that physiological concentrations of Dopamine, acting directly on T cells via well characterized Dopamine receptors, can modify numerous important T cell functions, such as, for example, β integrin binding, cytokine secretion and membrane depolarization. Whereas Dopamine effects on T cells have been previously considered secondary to other, classic T cell activating factors such as cytokines and LPS, the present invention surprisingly demonstrates that Dopamine acts directly to modulate specific gene expression, and upregulate cytokine activity in unstimulated, resting T cells. More surprisingly, while reducing the present invention to practice the present inventor has uncovered divergent Dopamine effects in stimulated, as opposed to resting T cells, demonstrating the prevention of T cell mediated autoimmune and allergic disease and modification of T cell commitment by Dopamine and Dopamine analogs. Thus, the present invention provides methods for the modulation of T-cell activity by Dopamine and specific Dopaminergic receptor functional analogs and, more particularly, methods for the treatment of bacterial, viral, fungal infectious and parasitic diseases, containment of auto-immune and other injurious inflammatory processes, inhibition and prevention of tumor growth and dissemination, and prevention of host rejection of engrafted tissue employing Dopaminergic receptor-mediated regulation of T-cell activation and apoptosis, cytokine secretion and integrin-binding activity, devoid of the above limitations.

### SUMMARY OF THE INVENTION

According to the present invention there is provided a method of regulating activity of a T-cell population, the method comprising exposing the T-cell population with a molecule selected capable of regulating a Dopamine receptor activity or the expression of a gene encoding a Dopamine receptor of T-cells of the T-cell population, thereby regulating Dopamine mediated activity in the T-cell population.

According to another aspect of the present invention there is provided a method of suppressing activity of a T-cell population, the method comprising exposing the T-cell population with a concentration of a molecule selected capable of upregulating a Dopamine receptor activity, said concentration sufficient to suppress T-cell function in the T-cell population.

According to yet another aspect of the present invention there is provided a method of regulating T-cell activity in a mammalian subject having abnormal T-cell activity, the method comprising providing to a subject identified as having the abnormal T-cell activity a therapeutically effective amount of a molecule selected capable of regulating a Dopamine receptor activity or an expression of a gene encoding said Dopamine receptor thereby regulating T-cell activity in the mammalian subject.

According to still another aspect of the present invention there is provided a method of treating or preventing a T-cell related disease or condition characterized by abnormal T-cell activity in a mammalian subject, the method comprising providing to a subject identified as having the T-cell related disease or condition characterized by abnormal T-cell activity a therapeutically effective amount of a molecule selected capable of regulating an activity of a Dopamine receptor or an expression of a gene encoding said Dopamine receptor, said amount being sufficient to regulate T-cell activity, thereby treating or preventing the T-cell related disease or condition in the mammalian subject.

According to further features in described preferred embodiments, the T-cell population is a resting T-cell population.

According to yet further features in described preferred embodiments, said Dopamine receptor is a D3 Dopamine receptor.

According to still further features in described preferred embodiments, said molecule is selected capable of upregulating said Dopamine receptor activity or said expression of said gene encoding said Dopamine receptor, thereby upregulating Dopamine mediated activity of said T-cells of the T-cell population or the subject.

According to further features in described preferred embodiments, said molecule is selected from the group consisting of Dopamine, an upregulating Dopamine analog, an upregulating anti Dopamine receptor antibody and an expressible polynucleotide encoding a Dopamine receptor.

According to yet further features in described preferred embodiments, said upregulating anti-Dopamine receptor antibody is a monoclonal or a polyclonal antibody.

According to further features in described preferred embodiments, the T-cell related disease or condition is a disease or condition characterized by suboptimal T-cell activity selected from the group consisting of congenital immune deficiencies, acquired immune deficiencies, infection, neurological disease and injury, psychopathology and neoplastic disease; and whereas said molecule is selected capable of upregulating an activity of a Dopamine receptor or an expression of a gene encoding said dopamine receptor.

According to still further features in described preferred embodiments, said expressible polynucleotide encoding a Dopamine receptor is designed capable of transient expression or within cells or stably integrating within the genome of cells of the T-cell population or T-cells of the subject.

According to yet further features described in preferred embodiments, the T-cell related disease or condition is a disease or condition characterized by excessive T-cell activity selected from the group consisting of autoimmune, allergic, neoplastic, hyperreactive, pathopsychological and neurological diseases and conditions, graft-versus-host disease, and allograft rejections and whereas said molecule is selected capable of downregulating an activity of a Dopamine receptor or an expression of a gene encoding said dopamine receptor.

According to further features in described preferred embodiments, said molecule is selected capable of downregulating said Dopamine receptor activity or said expression of said gene encoding said Dopamine receptor, thereby downregulating Dopamine mediated activity in the T-cell population or T-cells of the subject.

According to still further features in described preferred embodiments, said molecule is selected from the group consisting of a downregulating Dopamine analog, a downregulating anti Dopamine receptor antibody, a single stranded polynucleotide designed having specific Dopamine receptor transcript cleaving capability, an expressible polynucleotide encoding a ribozyme designed having specific Dopamine receptor transcript cleaving capability, a polynucleotide designed comprising nucleotide sequences complementary to, and capable of binding to Dopamine receptor transcripts, coding sequences and/or promoter elements and an expressible polynucleotide encoding nucleotide sequences complementary to, and capable of binding to Dopamine receptor transcripts, coding sequences and/or promoter elements.

According to still further features in described preferred embodiments, said expressible polynucleotide includes a sequence as set forth in any of SEQ ID NOs: 10-14.

According to further feature in described preferred embodiments, said step of providing said molecule is effected by systemic or local administration of said molecule to the subject, or providing said molecule to an ex vivo T-cell population and administering said ex vivo T-cell population to the subject.

According to yet further features in described preferred embodiments, regulating Dopamine mediated activity in the T cell population or the mammalian subject results in a change in at least one T cell activity selected from the group consisting of P-integrin binding, fibronectin adhesion, depolarization, cytokine secretion, proliferation, gene expression and induction of inflammatory disease.

According to further features in described preferred embodiments, further comprising the step of monitoring said at least one T-cell activity in the T-cell population or the T-cells of the subject.

According to yet further features in described preferred embodiments, said monitoring said at least one T-cell activity is effected by determining at least one parameter selected from the group consisting of β-integrin binding, fibronectin adhesion, depolarization, cytokine secretion, proliferation, gene expression and induction of inflammatory disease.

According to still further features in described preferred embodiments, the subject is suffering from a cancerous disease or condition characterized by excess T-cell activity, and the method further comprising the step of determining cancer cell proliferation and/or metastasis in the subject prior to and/or following said step of providing.

According to yet further features in described preferred embodiments, said cancerous disease or condition characterized by excess T-cell activity is a myeloproliferative disease.

According to further features in described preferred embodiments, the T-cell related disease or condition is a T-cell inflammatory disease or condition characterized by excessive T-cell activity, and whereas said molecule is a molecule selected capable of upregulating an activity of a Dopamine receptor, further comprising the step of exposing stimulated T cells from the subject to a therapeutically effective amount of said molecule selected capable of upregulating an activity of a Dopamine receptor, thereby suppressing said T cell inflammatory disease in the subject.

According to further features in described preferred embodiments, the method further comprising the step of monitoring a symptom of said T-cell inflammatory disease or condition in the subject prior to and/or following said step of providing.

According to still further features in described preferred embodiments, monitoring said T-cell activity is effected by determining an activity selected from the group consisting of, β-integrin binding, fibronectin adhesion, depolarization, cytokine secretion, proliferation, gene expression and induction of inflammatory disease.

According to further features in described preferred embodiments, the subject is suffering from a cancerous disease or condition characterized by excess T-cell activity, and the method further comprising the step of determining cancer cell proliferation and/or metastasis in the subject prior to and/or following said step of providing.

According to yet further features in described preferred embodiments, said cancerous disease or condition characterized by excess T-cell activity is a myeloproliferative disease.

According to further features in described preferred embodiments, the T-cell related disease or condition is a T-cell inflammatory disease or condition characterized by excessive T-cell activity, and whereas said molecule is a molecule selected capable of upregulating an activity of a Dopamine receptor, further comprising the step of exposing stimulated T cells from the subject to a therapeutically effective amount of said molecule selected capable of upregulating an activity of a Dopamine receptor, thereby suppressing said T cell inflammatory disease in the subject.

According to yet further features in described preferred embodiments, the method further comprising the step of monitoring a symptom of said T-cell inflammatory disease or condition in the subject prior to and/or following said step of providing.

According to still further features in described preferred embodiments, said T-cell inflammatory disease is selected from the group consisting of Delayed Type Hypersensitivity (DTH), Experimental Autoimmune Encephalomyelitis (EAE) and Multiple Sclerosis (MS).

According to still another aspect of the present invention, there is provided a method of suppressing activity of a T-cell population, the method comprising exposing the T-cell population with a concentration of a molecule selected capable of upregulating a Dopamine receptor activity, said concentration sufficient to suppress T-cell function in the T-cell population.

According to further features in described preferred embodiments, said molecule selected capable of upregulating a Dopamine receptor activity is Dopamine or a Dopamine analog and said concentration sufficient to suppress T-cell function is greater than 10⁻⁴ M.

According to yet further features in described preferred embodiments, said Dopamine receptor is a D3 Dopamine receptor.

According to another aspect of the present invention there is provided a population of T-cells suitable for treating or preventing a disease or condition characterized by abnormal T-cell activity in a subject, the population of T cells comprising T-cells characterized by modified sensitivity to Dopamine receptor stimulation, said T-cells being capable of treating or preventing a disease or condition characterized by abnormal T-cell activity upon administration to the subject.

According to further features in described preferred embodiments, said T-cells comprise an exogenous expressible polynucleotide sequence encoding expressing a Dopamine receptor.

According to yet further features in described preferred embodiments, said T-cells comprise an exogenous polynucleotide sequence capable of downregulating expression of a gene encoding a Dopamine receptor.

According to another aspect of the present invention there is provided an assay for determining the sensitivity of a resting T-cell population to regulation of Dopamine receptor activity, the assay comprising:(a) exposing the T-cell population to a molecule selected capable of regulating a Dopamine receptor activity or the expression of a gene encoding a Dopamine receptor, and(b) assessing a state of the T-cell population.

According to further features in described preferred embodiments, step (a) is effected by exposing the T-cell population to a range of concentrations of said molecule, and whereas step (b) is effected by assessing said state at each concentration of said range.

According to yet further features in described preferred embodiments, said Dopamine receptor is a D3 Dopamine receptor.

According to still further features in described preferred embodiments, said molecule is a molecule selected capable of upregulating said Dopamine receptor activity or said expression of said gene encoding said Dopamine receptor, thereby upregulating Dopamine mediated activity in the T-cell population.

According to further features in described preferred embodiments, said molecule selected capable of upregulating an activity of a Dopamine receptor or an expression of a gene encoding said dopamine receptor is selected from the group consisting of Dopamine, an upregulating Dopamine analog, an upregulating anti Dopamine receptor antibody and an expressible polynucleotide encoding a Dopamine receptor.

According to yet further features in described preferred embodiments, said upregulating anti-Dopamine receptor antibody is a monoclonal or a polyclonal antibody.

According to still further features in described preferred embodiments, said expressible polynucleotide encoding a Dopamine receptor is designed capable of transient expression within cells of the T-cell population.

According to further features in described preferred embodiments, said expressible polynucleotide encoding a Dopamine receptor is designed capable of stably integrating into a genome of cells of the T-cell population.

According to yet further features in described preferred embodiments, said expressible polynucleotide includes a sequence as set forth in any of SEQ ID NOs: 10-14.

According to still further features in described preferred embodiments, said molecule is a molecule selected capable of downregulating said Dopamine receptor activity or said expression of said gene encoding said Dopamine receptor, thereby downregulating Dopamine mediated activity in the T-cell population.

According to still further features in described preferred embodiments, said molecule selected capable of downregulating an activity of a Dopamine receptor or an expression of a gene encoding said dopamine receptor is selected from the group consisting of a downregulating Dopamine analog, a downregulating anti Dopamine receptor antibody, a single stranded polynucleotide designed having specific Dopamine receptor transcript cleaving capability, an expressible polynucleotide encoding a ribozyme designed having specific Dopamine receptor transcript cleaving capability, a polynucleotide designed comprising nucleotide sequences complementary to, and capable of binding to Dopamine receptor transcripts, coding sequences and/or promoter elements and an expressible polynucleotide encoding nucleotide sequences complementary to, and capable of binding to Dopamine receptor transcripts, coding sequences and/or promoter elements.

According to still further features in described preferred embodiments, said downregulating anti-Dopamine receptor antibody is a monoclonal or a polyclonal antibody.

According to further features in described preferred embodiments, step (b) is effected by determining an activity selected from the group consisting of β-integrin binding, fibronectin adhesion, depolarization, cytokine secretion, proliferation, gene expression and induction of inflammatory disease.

According to yet another aspect of the present invention there is provided an article of manufacture, comprising packaging material and a therapeutically effective amount of a pharmaceutical composition being identified for the treatment of a T-cell related disease or condition associated with abnormal T-cell activity, said pharmaceutical composition including a molecule selected capable of regulating an activity of a Dopamine receptor or an expression of a gene encoding said Dopamine receptor in T-cells and a pharmaceutically acceptable carrier.

According to further features in described preferred embodiments, said molecule is capable of upregulating an activity of a Dopamine receptor or an expression of a gene encoding said Dopamine receptor in T-cells and whereas the T-cell related disease or condition is a disease or condition characterized by suboptimal T-cell activity.

According to yet further features in described preferred embodiments, said molecule selected capable of upregulating an activity of a Dopamine receptor or an expression of a gene encoding said dopamine receptor is selected from the group consisting of Dopamine, an upregulating Dopamine analog, an upregulating anti Dopamine receptor antibody and an expressible polynucleotide encoding a Dopamine receptor.

According to further features in described preferred embodiments, said molecule is capable of downregulating an activity of a Dopamine receptor or an expression of a gene encoding said Dopamine receptor in T-cells and whereas the T-cell related disease or condition is a disease or condition characterized by excessive T-cell activity.

According to still further features in described preferred embodiments, said molecule selected capable of downregulating an activity of a Dopamine receptor or an expression of a gene encoding said dopamine receptor is selected from the group consisting of a downregulating Dopamine analog, a downregulating anti Dopamine receptor antibody, a single stranded polynucleotide designed having specific Dopamine receptor transcript cleaving capability, an expressible polynucleotide encoding a ribozyme designed having specific Dopamine receptor transcript cleaving capability, a polynucleotide designed comprising nucleotide sequences complementary to, and capable of binding to Dopamine receptor transcripts, coding sequences and/or promoter elements and an expressible polynucleotide encoding nucleotide sequences complementary to, and capable of binding to Dopamine receptor transcripts, coding sequences and/or promoter elements.

The present invention successfully addresses the shortcomings of the presently known configurations by providing, for the first time, methods and materials for modulation of T-cell activity by direct stimulation of T-cell Dopamine receptors, and for regulation of T-cell Dopamine receptor sensitivity.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A and 1B depict the Dopamine and Dopamine D3 receptor agonist DPAT induction of T-cell adhesion to fibronectin. In FIG. 1A, human T-cells were labeled with [⁵¹Cr], exposed to either Dopamine or DPAT (10 nM) and seeded in fibronectin-coated wells. The non-adherent T-cells were removed by thorough washings, while the adherent T-cells were lysed, and the radioactivity in the resulting supernatants determined. Dopamine-mediated induction of T-cell adhesion to fibronectin was compared to that of the selective Dopamine D3 receptor agonist 7-hydroxy DPAT, and to that of untreated cells. DPAT-treated T-cell adhesion to BSA-coated wells, and adhesion of untreated T-cells (controls) was <6%. In FIG. 1B, the specificity of the DPAT induction of fibronectin binding was demonstrated. A T-cell adhesion assay was performed in parallel in three different setups, in which either the T-cells, or the fibronectin-coated wells, or both, were transiently exposed to DPAT 10⁻⁸ M prior to assaying T-cell adhesion.

FIGS. 2A and 2B are graphic representations of the dose dependent nature of Dopamine and DPAT induction of T-cell adhesion to fibronectin. In FIG. 2A a clear optimum of Dopamine induction of T-cell binding is reached at 10 nM. FIG. 2B depicts a similar sensitivity of T-cells to DPAT induction of binding to fibronectin.

FIGS. 3A-C demonstrate the α₄β₁ and α₅β₁integrin-dependent nature of Dopamine and DPAT induction of T-cell binding to fibronectin. Normal human T-cells were pretreated with specific monoclonal antibodies directed against human integrin moieties α₄β₁and α₅β₁ (anti VLA-4, anti VLA-5 and anti-CD29), with specific peptides involved in integrins-fibronectin recognition (RGD-containing peptides), control antibodies directed against non-relevant integrin moieties (anti VLA-2 and anti LFA-1) and the non-relevant control RGE-containing peptide and then exposed to either 10 nM Dopamine (FIG. 3A), DPAT (FIG. 3B), or the potent T-cell activator PMA (FIG. 3C) and their adhesion to fibronectin determined. The results are presented as the mean ±SD CPM of bound T-cells from quadruplicate wells. One experiment, representative of two, is presented for each of the three inducers of T-cell binding. Both Dopamine, and DPAT mediated, as well as PMA induction of T-cell binding to fibronectin is consistently and specifically inhibited by the relevant monoclonal antibodies and competitive protein moiety.

FIGS. 4A-D depict the inhibition of Dopamine and DPAT-induced T-cell fibronectin binding by specific Dopamine receptor antagonists. Purified normal human T-cells were exposed to Dopamine (10 nM) in the absence or presence of increasing amounts of the D3-specific receptor antagonist U-Mal (10⁻⁸-10⁻⁶M) (FIG. 4A), D2/D1 receptor antagonist Butaclamol and D2 receptor antagonist Pimozide (10⁻⁸ M)(FIG. 4B), or D2/D1 receptor antagonist Haloperidol (10⁻⁸ M) (FIG. 4C), following which their adhesion to Fibronectin-coated wells was determined. FIG. 4D depicts similar inhibition of DPAT (10 nM) induction of T-cell binding to fibronectin by Dopamine receptor antagonists. Human T-cells were exposed to DPAT (10⁻⁸M) in the presence or absence of antagonists U-Mal, Butaclamol or Pimozide. Non-radioactive assessment of T-cell binding was performed as described in Material and Methods, results presented as the mean ±OD of bound T-cells from 4-6 wells. One representative experiment out of three is shown.

FIG. 5 demonstrates the induction of T-cell adhesion to fibronectin by Dopamine D3 and D2 receptor agonists. Purified normal human T-cells were exposed to DPAT, the D1/D2 agonist Pergolide or the D2-specific agonist Bromocryptine (all at 10⁻⁸M), and their adhesion to fibronectin coated wells was determined. The results are presented as the mean ±SD OD of bound T-cells from 4-6 wells. One experiment out of four is shown.

FIGS. 6A and 6B demonstrate the identification of Dopamine D3 receptor on human and mouse T-cell surface. Purified normal human (FIG. 6A) and mouse antigen-specific (Myelin Basic Protein, MBP87-99) (FIG. 6B) T-cells were reacted with rabbit anti-D3 Dopamine receptor antibody, followed by FITC-conjugated anti-rabbit Ig, and measurement of fluorescent intensity by FACS. Black outlined lined curves represent D3 receptor detection, grey shaded curves represent T-cells reacting with the control antibodies. One representative experiment out of 3 is presented.

FIGS. 7A and 7B demonstrate the depolarization of human and mouse T-cell depolarization by Dopamine D3 receptor agonist DPAT. Purified normal human (FIG. 7A) and mouse antigen-specific (Myelin Basic Protein, MBP87-99) (FIG. 7B) peripheral T-cells were incubated in serum-free RPMI, washed and resuspended in RPMI, loaded with the voltage-sensitive oxonol fluorescent dye di-BA-C₄ (300 nM), and exposed to DPAT (10⁻⁸ M). Black outlined curves represent the DPAT treated cells. Gray shaded curves represent the untreated controls. FIG. 7C depicts the depolarization of purified normal human peripheral T-cells loaded with the voltage sensitive dye and exposed to increasing concentrations (10-40 mM) of K⁺ in RPMI (black outlined curve) or normal (5 mM K⁺) RPMI for 30 minutes (grey shaded curve). Fluorescent intensity, indicating depolarization (abscissa) proportional to the membrane potential was measured by FACS.

FIGS. 8A and 8B demonstrate long-term supression of T-cell induced experimental autoimmune encephalomyelitis (EAE) by brief exposure to Dopamine D3 receptor agonist DPAT. Cultured mouse anti-myelin proteolipid T-cells (SJL/J anti PLP 139-151), stimulated 72 hours with antigen, were incubated 1 hour with DPAT (10⁻⁸ M)(+DPAT, solid squares) or fresh medium (open circles), washed, resuspended in PBS and inoculated intravenously into naive SJL/J recipient mice (6 mice per group) (FIG. 8A). FIG. 8B depicts abolition of the protective effect of DPAT by 5 minutes pretreatment of the encephalitogenic T-cells with the D3 Dopamine receptor antagonist U-Mal (10⁻⁷ M) (open triangles, dotted line is +U-Mal, +DPAT) prior to exposure to DPAT. Control mice received untreated, SJL/J anti PLP 139-151 T-cells (open circles, bold line). EAE was scored according to a scale of neuropathology from 0 (no abnormality) to 6 (death). One representative experiment from four is presented.

FIGS. 9A and 9B demonstrate supression of T-cell mediated experimental Delayed Type Hypersensitivity (DTH) by brief exposure to Dopamine D3 receptor agonist DPAT. Lymph nodes were removed from oxazalone-sensitized mice, and the T-cell suspension was exposed ex-vivo to increasing concentrations of DPAT (FIG. 9A, DPAT 10¹⁰-DPAT 10⁻⁶M) as described for FIG. 8 hereinabove. Control T-cells were incubated with medium only (untreated T-cells). The T-cells were then washed, resuspended in PBS and injected intravenously into naive syngeneic mice. The ears of the recipient mice were than challenged with oxazalone, and the degree of ear swelling was measured by micrometer 24 hr later. DTH is expressed as the mean ±SD of ear swelling in units of 10⁻² mm. FIG. 9B depicts abolition of the protective effect of DPAT by 5 minutes pretreatment of the oxazalone-sensitized T-cells with the D3 Dopamine receptor agonist U-Mal (10⁻⁷ M) (FIG. 9B, +U-Mal, +DPAT) prior to exposure to DPAT. Oxazalone-sensitized T-cells were incubated ex vivo for one hour with either DPAT (10⁻⁸ M, +DPAT) or U-Mal (10⁻⁷ M, +U-Mal), or both. The T-cells were then washed, resuspended in PBS, injected intravenously into normal recipients, and DTH measured as described hereinabove. Note the significant reduction of DTH (>60%) with the Dopamine receptor agonist DPAT, and the near total block of protection by the D3 antagonist U-Mal. FIGS. 9A and 9B represent two individual experiments, each experimental group consisting of 8 mice.

FIGS. 10A and 10B illustrate the induction by Dopamine of cytokine IL-10 secretion in normal human peripheral T-cells. Freshly separated human peripheral T-cells were incubated in T-cell medium for 72 hours with 10 nM Dopamine (FIG. 10A, Dopamine) or no addition (FIG. 10B, Untreated), and levels of the cytokine IL-10 were measured in the supernatants by a qualitative sandwich ELISA, as described in Materials and Methods. The results are expressed as pg/ml IL-10. Note a 2.5 fold increase in IL-10 secretion with Dopamine. FIG. 10B shows fluorescent profiles of FACS analysis of normal human peripheral T-cells after 48 hours incubation with (FIG. 10B Dopamine 10⁻⁸M) or without (FIG. 10B Untreated) Dopamine. Immunostaining is with intracellular anti-IL-10 FITC conjugated (FL-1) and anti-CD4 PE conjugated (FL-2) antibodies, as described in Materials and Methods hereinbelow. Note the Dopamine-induced increase (9%) in the total number of IL-10 producing T-helper cells (right upper quadrant, IL-10+, CD4+).

FIGS. 11A-C illustrate the induction by Dopamine of cytokine TNFα, but not IFNγ or IL-4 secretion in normal human peripheral T-cells. Freshly separated human peripheral T-cells were incubated in T-cell medium for 24 hours with 10 nM Dopamine (FIGS. 11A-C, Dopamine) or no addition (FIGS. 11A-C, Untreated), and levels of the cytokines TNFα, IFNγ and IL-4 were measured in the supernatants by a qualitative sandwich ELISA, as described in Materials and Methods. The results are expressed as pg/ml. Note the significant increase in only TNFα secretion with Dopamine.

FIGS. 12A-E demonstrate the time dependent induction by Dopamine of cytokine TNFα and IL-10 secretion in normal human peripheral T-cells. Freshly separated human peripheral T-cells were incubated in T-cell medium with 10 nM Dopamine (FIGS. 12A-E, Dopamine) or no addition (FIGS. 12A-E, Untreated), for 24 (FIG. 12A), 48 (FIGS. 12B and 12D) or 72 (FIGS. 12C and 12E) hours and levels of the cytokines TNFα and IL-10 (as indicated) were measured in the supernatants by a qualitative sandwich ELISA, as described in Materials and Methods. The results are expressed as pg/mi. Note the maximal effect of Dopamine on TNFα secretion after 24 and 48 hours (FIGS. 12B and 12C), and on IL-10 secretion after 72 hours (FIG. 12E).

FIGS. 13A and 13B demonstrate the dose dependent induction by Dopamine of cytokine TNFα and IL-10 secretion in normal human peripheral T-cells. Freshly separated human peripheral T-cells were incubated in T-cell medium with increasing concentrations (as indicated) of Dopamine (FIGS. 13A and 13B, Dopamine conc.) or no addition (FIGS. 13A and 13B, 0), for 24 (FIG. 13A, TNFα) or 72 (FIG. 13B, IL-10) hours and levels of the cytokines TNFα and IL-10 were measured in the supernatants by a qualitative sandwich ELISA, as described in Materials and Methods. The results are expressed as pg/ml. Note the significant effect of Dopamine on both TNFα and IL-10 secretion from 10⁻⁸M, with maximal secretion induced by 10⁻⁴M Dopamine.

FIGS. 14A-14C illustrate the induction by Dopamine of “typical” and “atypical” cytokine secretion in resting cloned human T-cells. Cloned resting Th0 (FIG. 14A), Th2 (FIG. 14B) and Th1 (FIG. 14C) cells were incubated for 72 hours with 10 nM Dopamine (Dopamine) or no addition (Untreated), and levels of the cytokine IL-10 were measured in the supernatants by a qualitative sandwich ELISA, as described in Materials and Methods. The results are expressed as pg/ml IL-10. Note that Dopamine not only triggered secretion of the characteristic Th2 cytokine IL-10 from Th0 and Th2 clones (FIG. 14A and 14B, typical cytokine secretion), but also from a Th1 clone (FIG. 14C atypical, “forbidden” cytokine secretion).

FIGS. 15A and 15B illustrate the Dopamine receptor specificity of Dopamine induction of cytokines TNFα and IL-10 secretion in resting human peripheral T-cells. Freshly separated human peripheral T-cells were incubated in T-cell medium with 10⁻⁷ M receptor-specific Dopamine agonists (Dopamine agonist) or no addition (Untr.), for 72 (FIG. 15A) and 24 (FIG. 15B) hours, and levels of the cytokines TNFα and IL-10 (as indicated) were measured in the supernatants by a qualitative sandwich ELISA, as described in Materials and Methods hereinbelow. Dopamine agonists used are: SKF 38393 (D1), Quinpirole (D2), 7-OH-DPAT (D3) and PD-168077 (D4). The results are expressed as pg/ml. Note that whereas IL-10 secretion was induced by the D2 and D3 but not D1 or D4 receptors agonists (FIG. 15A, black arrows), TNFα secretion was induced by D1 and D2, but not D3 and D4, receptor agonists (FIG. 15B, black arrows).

FIGS. 16A and 16B illustrate receptor specificity of Dopamine antagonist inhibition of Dopamine-induced cytokines TNFα and IL-10 secretion in resting human peripheral T-cells. Freshly separated human peripheral T-cells were incubated in T-cell medium with 10⁻⁸ M Dopamine (no ant.), Dopamine and receptor-specific Dopamine antagonists (ant.), or no addition (Untr.), for 72 (FIG. 16A) and 24 (FIG. 15B) hours, and levels of the cytokines TNFα and IL-10 (as indicated) were measured in the supernatants by a qualitative sandwich ELISA, as described in Materials and Methods hereinbelow. Dopamine receptor antagonists used are: L-741,626 (D2 antagonist, 10⁻⁷ M), U-99194A maleate U-Mal (D3 antagonist, 10⁻⁶ M) and L-741,741 (D4 antagonist, 10⁻⁶ M). The results are expressed as pg/ml. Note that whereas IL-10 secretion was inhibited by the D2 and D3 but not D3 receptors antagonists (FIG. 16A, black arrows), TNFα secretion was inhibited by D3, but not D2 and D4, receptor antagonists (FIG. 16B, black arrows).

FIGS. 17A-E illustrate the effect of excess Dopamine on survival of normal human peripheral T-cells and Jurkat T-cell leukemia cells. Freshly separated human peripheral T-cells were incubated in T-cell medium with or without (0) increasing concentrations (10⁻⁶ to 10⁻³ M) of Dopamine and levels of the cytokines IL-10 (FIG. 17A) and TNFα (FIG. 17B) were measured in the supernatants by a qualitative sandwich ELISA, as described in Materials and Methods hereinbelow. Note the drastic reduction of cytokine secretion at 10⁻³ M Dopamine. FIG. 17C shows the PAGE separation of total ethidium-bromide stained RNA extracted from cells receiving similar exposure to high concentrations of Dopamine. Note the near total absence of r-RNA bands in cells treated with excess (10⁻³) Dopamine, indicating loss of viability. FIGS. 17D and 17E show the dose dependent induction of T-cell apoptosis by excess Dopamine. Jurkat T-cell leukemia cells (FIG. 17D) and normal human peripheral T-cells (FIG. 17E) were incubated with indicated concentrations (10⁻⁸ to 10⁻³ M) Dopamine as described hereinabove, and cell survival rate was determined. Note that high concentrations (2.5×10⁻⁴ to 10⁻³ M) of Dopamine dramatically impaired T-cell survival rates.

FIGS. 18A-C depict the proliferation of resting normal human and cultured Jurkat T-cells induced by physiological concentrations of DPAT. Purified normal human T-cells or Jurkat T-cell leukemia cells were suspended in supplemented RPMI-1640 (Beit-Haemek, Israel), seeded in 96 round bottom microtiter wells (Greiner, Nurtingen, FRG) and exposed to DPAT as indicated (+DPAT 10⁻⁸M to +DPAT 10⁻¹⁰M) for 48 hours at 37° C. Numbers of T-cells were evaluated using the CyQuant cell proliferation assay kit (Molecular Probes), according to the manufacturer instructions. FIG. 18A represents a calibration of the CyQuant cell kit, showing the linear relationship between the fluorescent intensity and untreated human T-cell number. Note the similar maximal enhancement of T-cell proliferation with 10⁻¹⁰M DPAT in purified normal human (18B) and cultured Jurkat T-cells (FIG. 18C).

FIGS. 19A-C depicts the detection of D3 Dopamine receptor expression in T-cells by RT-PCR. FIG. 19A shows the structure of the mouse Dopamine D3 receptor gene, indicating the position of primers used for PCR (TM, transmembrane domain) amplification. The 63 base pairs (bp) addition correspond to 21 amino acids present in the long D3 (D3L) and absent from the short D3 (D3S) receptor. FIG. 19B shows the electrophoretic separation of specific RT-PCR amplification products from a mouse antigen (MBP 87-99)-specific T-cell line; PCR was performed using the following oligonucleotide primers, representing different regions of the mouse D3 dopamine receptor, previously cloned from mouse brain: A (5′ CTCTCTCCTGGCCAGACACAT-3′)(SEQ ID NO: 1) and B (5′-AGAGAAGAAGGCCACCCAG-3′)(SEQ ID NO:2), corresponding to nucleotides 867-887 and 1107-1125 respectively; C (5′-GGAGTCTGGAATTTCAGC-3′)(SEQ ID NO:3) and D (5′-CCTTTGCCTCAGGACCATGTA-3′)(SEQ ID. NO:4) corresponding to nucleotides 280-297 and 634-654 respectively; E (5′ gGAATTCC TCTGTGTGGGCCATG-3′)(SEQ ID NO:5) and F (5′-ACGTCGACAGGAGC TCTG C-3′)(SEQ ID NO:6) corresponding to nucleotides-18-+3 and 429-447 respectively. FIG. 19C shows independent replicates of PCR amplification products performed as in 19B, using primers G (5′-gga attCCAGGTTTCTGTCAGATGCC-3′)(SEQ ID NO:7) and H (5′ ggaattCCGTTGCTGAGTTTTCGAACC-3′)(SEQ ID NO:8), corresponding to nucleotides 770-789 and 1029-1049 respectively. The amplification products were separated on 2% agarose gel and stained by ethidium bromide. Each lane represents different RNA cell extraction. Arrows point to the ˜300 bp and ˜230 bp of the long and short forms of the D3 Dopamine receptor respectively. Size is determined in reference to molecular weight DNA (1 kb) markers (19B and 19C, M). Negative controls, omitting either reverse transcriptase or template (FIG. 19B, DDW), did not show any visible signal, excluding the possibilities of a signal due to genomic DNA or cross-contamination of samples.

FIGS. 20A and 20B are tables depicting the upregulation (FIG. 20A) and downregulation (FIG. 20B) of specific genes in T-cells by the Dopamine D3 receptor specific agonist DPAT. Bolded genes are of specific interest.

### DESCRIPTION OF THE PREFERRED EMBODIMENTS

The present invention is of methods which can be used for modulation of T-cell activity. Specifically, the present invention relates to the use of Dopamine, specific Dopaminergic receptor functional analogs, and modulation of T-cell Dopamine receptor function in the treatment of bacterial, viral, fungal infectious and parasitic diseases, containment of auto-immune and other injurious inflammatory processes, inhibition and prevention of psychopathology, neoplastic allergic and neurogenic diseases and conditions, and prevention of host rejection of engrafted tissue employing Dopaminergic receptor-mediated regulation of T-cell activation, cytokine secretion, proliferation, apoptosis and integrin- binding activity.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

At any given moment, T-cell populations throughout the body have to carry out a myriad of different activities, among them patrolling and surveillance, helping and suppressing, combating and killing. Moreover, T-cell activities must be precisely regulated and coordinated with many other cell types in general, and, perhaps most importantly, with dynamic neuro-endocrine networks. It is difficult to conceive that all these tasks are mediated solely via the ‘classical’ immunological interactions between the T-cell receptor (TCR), the principal receptor of these cells, and specific antigens, even if assisted by other immunological molecules, such as cytokines and chemokines and their receptors. In fact, the factors responsible for regulating T-cell activities within immune privileged environments, such as the brain, are still unknown and their discovery will certainly have important implications for the understanding and treatment of various T-cell mediated CNS pathologies, such as the autoimmune T-cell mediated multiple sclerosis.

Can T-cells respond directly to neuroendocrine molecules, despite the conceptual dogma of a ‘language’ barrier between effector molecules used for communication within the nervous, endocrine and immune systems? No doubt that such a direct mode of communication could be of great benefit for coordinating body functions in numerous physiological and pathophysiological conditions. The present inventors addressed this question by investigating whether T-cells can be directly activated by the potent catecholamine neurotransmitter Dopamine.

T-lymphocytes most probably encounter Dopamine in vivo, when the latter neurotransmitter is released from nerves supplying the spleen, thymus, lymph nodes, bone marrow and other T cell-enriched tissues. It has been demonstrated that Dopamine binds to T-cells, that L-Dopa and Dopamine suppress mitogen-induced proliferation of human peripheral blood mononuclear cells (PBMC) and that bromocriptine (a Dopamine D2 agonist) decreases B and T lymphocytes proliferation, and the severity of the T-cell mediated disease -experimental autoimmune encephalomyelitis (EAE) [22]. Catecholamines are synthesized by lymphocytes, and are suggested to function in an autocrine loop capable of down-regulating the lymphocytes own function. However, these studies have not demonstrated the presence of functional Dopamine receptors on T-cells, or suggested a role that Dopamine may play in triggering T-cell function.

While reducing the present invention to practice, it was demonstrated, for the first time, that Dopamine interacts directly and functionally with specific Dopaminergic receptors on normal human T-cells, resulting in activation of the β1 integrin moieties, strong integrin-mediated adhesion to a major ECM glycoprotein (fibronectin), induction of “typical” and “forbidden” cytokine secretion profiles, up- and downregulation of specific T-cell genes, depolarization of T-cell membranes, induction of T-cell apoptosis and suppression of pathological T-cell immunoreactivity. In the context of the present invention, and the results presented herein in the Examples section, it is proposed that Dopamine can directly affect the activation, immune reactivity, cytokine profile, migration and extravasation of T-cells across blood vessels and tissue barriers in a variety of biological and pathological settings, among them inflammation and autoimmune diseases. These proposed effects of Dopamine may be especially relevant for T-cells which, when patrolling the brain, are in a constant need to ‘sense’ nerve-secreted stimuli and to respond to them by readjusting their secretory, immune reactive, adhesive and migratory behavior. Thus, under normal conditions, Dopamine may lead to beneficial activation and migration of T-cells towards resting, inflamed, injured or stressed tissues, and may serve to direct neural coordination of immune function. Furthermore, under conditions of undesirable T-cell migration and function (autoimmune disease, chronic inflammation, allergic conditions, graft-versus-host disease, and allograft rejection) Dopamine may have detrimental effects and may be a target for immunosuppression.

Of great importance is the understanding that Dopamine effects in T cells are context-dependent. While reducing the present invention to practice, it was uncovered that Dopamine, and Dopamine functional analogs have a different, sometimes opposite effect on resting versus activated T-cells. Thus, for example, the same physiological concentrations of Dopamine stimulate cytokine secretion and integrin binding in resting T cells (Examples III, IV and VIII), but suppress T cell mediated autoimmune and allergic responses to activated T cells (Example VII).

Further, in the context of the present invention, it is proposed that one of the first rapid events taking place in any emergency situation is the recruitment of patrolling T-cells into injured, stressed or inflamed CNS loci via a neurotransmitter-evoked activation of the cell adhesion receptors (primarily the integrins), and the subsequent adhesion of these activated T-cells to their ligands on opposing cells and matrices. The results presented herein in the Examples section indicate, surprisingly, that Dopamine contributes to such a scenario.

While reducing the present invention to practice, it was also demonstrated, for the first time, that Dopamine interacts directly and fuinctionally with T-cells and conveys an activating effect on cytokine IL-10 and TNFα secretion, proliferation, membrane depolarization, and a suppressive effect on the immune reactivity of activated and sensitized T-cells. In addition, it was demonstrated that Dopamine conveys these activating effects by stimulating specific T-cell Dopamine receptor subtypes. For example, as detailed in the Examples section hereinbelow, membrane depolarization, IL-10 secretion and downregulation of immune reactivity is mediated via D2 and D3 receptors, whereas TNFα secretion is mediated predominately via type D1 and D3 receptors.

The D2-like Dopaminergic receptors (mainly the D2 and D3) show different but overlapping tissue distribution, and their amino acid sequences display a significant degree of conservation, particularly in the transmembrane domains where ligand-binding is thought to occur. The D2-like receptors on various target cells bind to the same type of ligands but with different order of potency.

In the adult organism, the Dopamine D2 receptors are the most abundant of all the D2-type receptors, being expressed most highly in the striatum and olfactory tubercules (correlating to the major Dopaminergic projection areas), as well as the substantia nigra and ventral tegmental areas (implying a role in presynaptic and postsynaptic structures), and in the pituitary (where it may be involved in the Dopaminergic control of prolactin secretion).

The Dopamine D3 receptors are expressed predominantly in limbic areas, including the olfactory tubercule, nucleus accumbens and hypothalamus. The distribution profile of the D3 receptor has led to suggestions that this subtype may be concerned with the Dopaminergic control of cognitive and emotional functions, and therefore may be involved in the pathogenesis of emotive disorders.

In the context of the present invention, it is important to note the recent demonstration of abnormal levels of Dopamine receptors on lymphocytes in a number of neurogenic conditions, such as schizophrenia, Parkinson's disease, Alzheimers disease and migraine. Demonstration of elevated D3 receptor mRNA levels in lymphocytes from human schizophrenic patients, decreased D3 Dopamine receptor mRNA levels in PBLs from Parkinson's patients, reduced density of Dopamine D2-like receptors in PBLs of Alzheimer's patients and increased RNA levels of the Dopamine D3, D4 and D5 receptors in. PBLs of migraine patients have lead to use of Dopamine binding as a peripheral diagnostic marker for the respective central nervous system diseases. Similarly, the structural alterations of Dopamine receptors found in the tissues of schizophrenics have been proposed as a basis for diagnostics and drug development (see, for example, U.S. Pat. No. 5,738,998 to Deth). The results presented herein indicate, however, that abnormal Dopamine receptor levels on lymphocytes in the abovementioned and other neurogenic pathologies may reflect modulations of lymphocyte (specifically T-cell) functionality and abnormal responsiveness to the neurotransmitter Dopamine.

In the context of the present invention, it is important to note the role of immune function in general, and T-cells in particular, in neuroprotective immunity. Activated T-cells in sufficient numbers, at crucial locations in the CNS, and with appropriate temporal coordination, are necessary for optimal healing following neuronal injury or viral infection of the CNS (Yoles E et al J Neurosci 2000;21:3740-8; and Binder G K and Griffen D E Science 2001;293:303-6). Thus, the compositions and methods of the present invention can be used for treatment and prevention of neuronal damage in CNS injury and infection.

The present invention provides methods and materials for Dopamine-mediated regulation of T-cell function via modulation of Dopamine receptor activation and sensitivity. These methods can be used to treat or prevent conditions resulting from suboptimal or excessive T-cell function.

Thus, according to the present invention there is provided a method of regulating activity of a T-cell population, the method comprising exposing the T-cell population to a molecule selected capable of regulating a Dopamine receptor activity or the expression of a gene encoding a Dopamine receptor of T-cells of the T-cell population, thereby regulating Dopamine mediated activity in the T-cell population.

Further according to the present invention there is provided a method of regulating T-cell activity in a mammalian subject having abnormal T-cell activity, the method comprising providing to a subject identified as having the abnormal T-cell activity a therapeutically effective amount of a molecule selected capable of regulating a Dopamine receptor activity or an expression of a gene encoding said Dopamine receptor thereby regulating T-cell activity in the mammalian subject.

Still further according to the present invention there is provided a method of treating or preventing a T-cell related disease or condition characterized by abnormal T-cell activity in a mammalian subject, the method comprising providing to a subject identified as having the T-cell related disease or condition characterized by abnormal T-cell activity a therapeutically effective amount of a molecule selected capable of regulating an activity of a Dopamine receptor or an expression of a gene encoding said Dopamine receptor, said amount being sufficient to regulate T-cell activity, thereby treating or preventing the T-cell related disease or condition in the mammalian subject.

Diseases or conditions related to T-cell deficiency or dysfunction would require upregulation of T-cell function, by Dopamine or Dopamine analogs possessing agonist or stimulatory properties. Although therapeutic use of Dopamine and agonist analogs of Dopamine has been previously proposed (Tsao, C W et al. Life Sci 1997;61(24): PL 361-71; Tsao, C W et al. Life Sci 1998; 62(21):PL 335-44; Morikawa, K. et al. Clin Exp Immunol 1994; 95(3); 514-8; Blank M. et al, Cell Immunol 1995 15; 162(1):114-22; U.S. Pat. No. 5,658,955 to Hitzig; U.S. Pat. Nos. 5,905,083, 5,872,133, 5,873,127, and 5,696,128 to Cincotta; U.S. Pat. No. 6,300,365 to Holman and U.S. Pat. No. 6,300,329 to Mclean, et al), the disclosed applications have been based on the non-specific stimulation of immune suppression, or the agonist's suppression of prolactin secretion. McLean, et al (U.S. Pat. No 5,658,955), and Hitzig (U.S. Pat. No 6,300,329) disclose the combination of serotonin and Dopamine agonist activity for treatment of neurogenic disorders and generalized immune suppression, respectively. No mention has been made of direct stimulation of T-cell activity, T-cell depolarization, T-cell apoptosis or neuro-immune effects via β-integrin regulation, specific gene activation or cytokine secretion.

The methods of the present invention can be used to upregulate T cell activity in conditions characterized by sub-optimal T-cell function. Thus, in one embodiment of the invention, the molecule selected capable of regulating the expression of the Dopamine receptor activity or expression of the gene encoding a Dopamine receptor is an upregulating molecule causing increased T-cell activity.

The upregulating molecule can be, for example, an upregulating Dopamine analog, Dopamine, an upregulating anti-Dopamine receptor antibody, or an expressible polynucleotide encoding a Dopamine receptor. The upregulating Dopamine analog may be a naturally occurring, or synthetic analog. In one preferred embodiment of the present invention, the upregulating Dopamine analog is a specific D3 receptor agonist, 7-OH-DPAT (DPAT). Commercially available upregulating Dopamine analogs suitable for use in the compounds and methods of the present invention may include, but are not limited to SKF 38393 (DI-specific), Quinpirole (D2-specific), and PD-168077 (D4-specific) (see: Research Biochemicals Incorporated, Nattick, Mass., USA).

In one preferred embodiment, manipulation of Dopamine receptor activity is used to regulate T-cell activity in a mammalian subject having abnormal T-cell activity, wherein the abnormal T-cell activity is suboptimal. This is effected by providing to the subject a therapeutically effective amount of an upregulator of Dopamine receptor activity or an expression of a gene encoding a Dopamine receptor. In the method of the present invention, the upregulating molecule may be administered in vivo, by administration to the subject via intravenous, parenteral, oral, transdermal, intramuscular, intranasal or other means or ex vivo, after removal of T-cells from the body and their isolation.

T-cells may be isolated from the blood by procedures known to one skilled in the art (see, for example, the Materials and Methods section that follows).

A specific example of such ex vivo treatment of immune cells for activation and therapeutic readministration may be found in Intn'l Pat. No. WO9950393A2 and A3 to Wank, although the methods described differ significantly from the methods disclosed herein. Wank describes the isolation and in vitro activation of peripheral blood mononuclear cells (phagocytes) from patients suffering from brain-related diseases, disorders and damage, including psychoses, autism, schizophrenia and developmental disturbances. In a report documenting adoptive immunotherapy of patients suffering from bipolar disorder, schizophrenia or autism, Wank describes similar in-vitro activation, and reintroduction of the patients' own T-cells, in order to combat “chronically infected”, understimulated lymphocytes thought associated with these disorders. In this form of therapy, the T-cells are not stimulated directly, rather via monoclonal antibodies against the CD3 polypeptide complex, and IL-2. The patients were required to endure numerous weekly treatments (up to 104 weeks in one patient), and although improvement in some symptoms was noted, additional therapies were continued during and after these trials of adoptive immunotherapy. No mention is made of direct stimulation of T-cells with neurotransmitters, of specific T-cell response to therapy, or of treatment with Dopamine, Dopamine analogs or other upregulators of T-cell Dopamine receptor activity.

Thus, according to one aspect of the present invention, there is provided a population of T-cell suitable for treating or preventing a disease or condition characterized by abnormal T-cell activity in a subject, the population of cells comprising T-cells characterized by modified sensitivity to Dopamine receptor stimulation. Such a population of T-cells can be used for treating or preventing a disease or condition characterized by abnormal T-cell activity upon administration to the subject. In one preferred embodiment, the sensitivity to Dopamine stimulation is modified by an exogenous expressible polynucleotide sequence encoding a Dopamine receptor, imparting increased sensitivity to Dopamine. Administration of a population of such sensitized T-cells can be beneficial in conditions of suboptimal T-cell activity, such as immunodeficiency, infection, neurological disease, injury and the like. In another preferred embodiment, the expressible polynucleotide sequence is capable of downregulating expression of a gene encoding a Dopamine receptor, such as a ribozyme or antisense polynucleotide. Administration of such desensitized T-cells can be beneficial in conditions and diseases of excess T-cell activity, such as autoimmune, allergic, pyschopathological (see example described hereinabove) neurological disease and the like. Suitable polynucleotides, and methods for their use in the present invention, are described in detail hereinabove. Additional methods for ex vivo treatment, selection, expansion and culturing of T-cells for readministration are well known in the art (see, for example, U.S. Pat. No. 6,451,316 to Srivatava).

Intracellular levels of Dopamine signal transducers may be manipulated by increasing the abundance of Dopamine receptor transcripts available for protein synthesis. This may be accomplished by introducing into target cells polynucleotides operatively coding for Dopamine receptor polypeptides. Delivery of such polynucleotides may be by injection, introduction into the circulation, or introduction into the body cavities by inhalation or insufflation. The expressible polynucleotides may be DNA or RNA sequences encoding a Dopamine receptor molecule, capable of enhancing Dopamine stimulation of target cells. Expression may be transient and reversible, or the polynucleotide may become integrated into the host genome, producing stable expression of the therapeutic polynucleotide. For illustrative methodology relating to the introduction of DNA and RNA sequences into host cells, see, for example, U.S. Pat. Nos. 5,589,466 and 6,214,806, both to Feigner et al.

Thus, according to one aspect of the present invention there is provided a method of upregulating T-cell activity in a T cell population or a mammalian subject, the method effected by introducing into the cells an expressible polynucleotide encoding a Dopamine receptor, the expressible polynucleotide designed capable of enhancing Dopamine receptor expression in said T-cells, thereby upregulating T-cell activity within cells of the T-cell population or mammalian subject. The expressible polynucleotides may contain sequences representing coding sequences of the Dopamine receptors, at least 60%, preferably at least 70%, more preferably at least 80%, more preferably at least 90% and most preferably about 100% homologous to any of SEQ ID NOs: 10-14. Methods for transformation of T-cells with expressible polynucleotides are described in detail hereinbelow.

Cell surface receptors may be targeted by specific antibodies, binding to epitopes exposed to the cellular environment. Although these antibodies may block ligand-receptor interaction, in binding some may also activate signal transduction pathways, behaving as agonists: this is commonly seen in autoimmune disease, such as Graves disease (for example, see Grando, S A. Antireceptor activity in pemphigus. Dermatology 2000; 201(4) 290-295; and Mijares, A., Lebesque, D., Walluk G. and Hoebeke, J. From agonist to antagonist. Mol. Pharmacol. August 2000 58 (2): 373-378). Similarly, specific antibodies directed against T-cell Dopamine receptors may act as agonists, stimulating T-cell activity.

Thus, in one embodiment of the present invention the upregulating molecule is an upregulating anti-Dopamine receptor. T-cells may be exposed to the antibody in vivo or isolated from the organism and exposed ex vivo (for methods of T-cell activation in vitro see, for example, the in-vitro assay of T-cell adhesion to fibronectin described in Materials and Methods section below, and assays of cytokine secretion described in Levite, M. et al, J Exp Med 2000, 191, 1167-76).

As is used herein, the term “antibody” refers to either a polyclonal or monoclonal antibody, recognizing at least one epitope of a Dopamine receptor. The present invention can utilize serum immunoglobulins, polyclonal antibodies or fragments thereof, (i.e., immunoreactive derivative of an antibody), or monoclonal antibodies or fragments thereof.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference).

As used in this invention, the term “epitope” means any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. -Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13, 65-93 (1995).

In a preferred embodiment of the present invention, the anti-Dopamine receptor antibody is a specific rabbit polyclonal antibody prepared against the D3 Dopamine receptor (Calbiochem, San Diego, Calif.).

The method of the present invention wherein the T-cell related disease or condition is a disease or condition characterized by suboptimal T-cell activity selected from the group consisting of congenital immune deficiencies, acquired immune deficiencies, infection, neurological disease and injury, psychopathology and neoplastic disease; and whereas said molecule is selected capable of upregulating an activity of a Dopamine receptor or an expression of a gene encoding said dopamine receptor. Immune deficient diseases or conditions that can be treated by upregulation of Dopamine-mediated T-cell activity of the methods of the present invention include congenital and acquired primary immunodeficiencies, such as the acquired immunodeficiency syndrome (AIDS), DeGeorge's syndrome, severe combined immunodeficiency; and secondary immunodeficiencies, such as anergy from tuberculosis, drug-induced leukopenia, non-HIV viral illnesses leukopenia, radiation poisoning, toxin exposure, malnutrition, and the like. Of special significance are neurogenic diseases and conditions in which increased T-cell activity may be beneficial, such as Parkinson's and Alzheimer's Disease. Similarly, neoplastic disease or conditions resulting from failure of immune surveillence, and bacterial, fungal, viral and parasitic infections may respond to upregulation of protective T-cell function by Dopamine, agonist (upregulating) Dopamine analogs, upregulating anti-Dopamine receptor antibodies, and expressible polynucleotides encoding a Dopamine receptor.

It will be appreciated that when treating such immune deficient conditions, dosage and treatment protocols are often determined according to severity of the disease or condition, co-existing complicating diseases or health factors, age, etc., and the subject's individual response to Dopamine-mediated upregulation of T-cell activity. In one specific example, T-cells are isolated from the patient prior to treatment (as detailed in the Examples section hereinbelow) and tested for cytokine secretion profiles, fibronectin adhesion and/or proliferation. Response to ex vivo treatment of T-cell with specific upregulators of Dopamine receptor activity, such as D3 receptor agonist DPAT, is then monitored within 48 hours of administration, and periodically until normalization of T-cell function and abatement of immune hypofunction is achieved. Thus, in one preferred embodiment, upregulating T-cell activity in the subject results in a change in at least one T-cell activity such as β-integrin binding, fibronectin adhesion, depolarization, cytokine secretion, proliferation and induction of inflammatory disease, which is monitored in T-cells of the subject.

In the context of the present invention, it is important to note the contribution of immune system dysfunction to aging processes. Altered signal transduction and aberrant cytokine production has been demonstrated in T-cells of elderly individuals, and aging T-cells are more susceptible to apoptosis (Pawelec, G. and Solana, R. Immunoageing-the cause or effect of morbidity? Trends in Immunol. 2001: July 22(7) 348-9). Thus, upregulation of T-cell function by Dopamine, agonist Dopamine analogs, upregulating anti-Dopamine receptor antibodies and expressible polynucleotides encoding a Dopamine receptor may be used to treat immune-related symptoms and processes of aging.

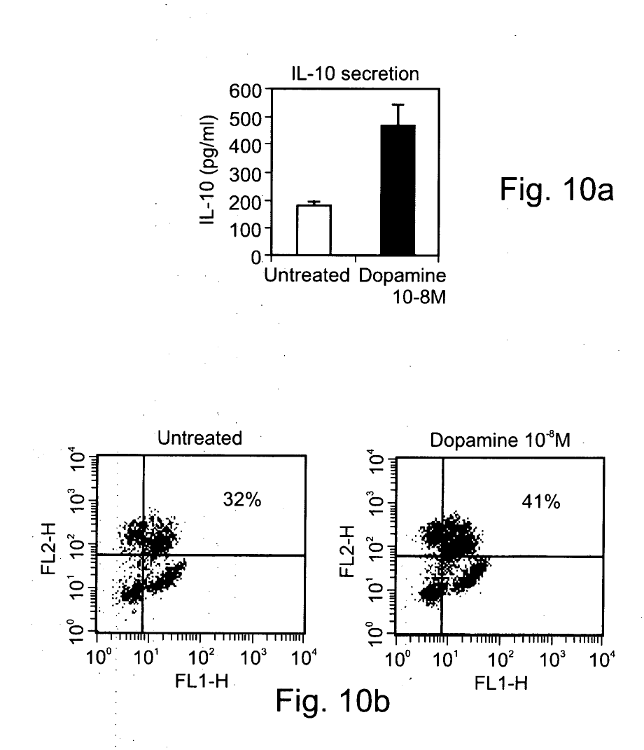

The present invention can be used to stimulate IL-10 secretion in T-cells. While reducing the present invention to practice, it was demonstrated, for the first time, that T-cells incubated with physiological concentrations (10⁻⁸ M) of Dopamine and Dopamine receptor agonists secreted significant amounts of the immunosuppressory cytokine IL-10 (FIGS. 10 and 12-16). IL-10 has been proposed for treatment for a wide variety of inflammatory and other diseases (as detailed in the Background section hereinabove). However, systemic administration of IL-10-inducing drugs such as cyclophosphamide is associated with intolerable side effects such as nausea, alopecia and infertility, and the feasibility of treatment with recombinant IL-10 is severely hampered by insufficient experience, short supply and high cost. Thus, induction in T-cells of endogenous IL-10 secretion by Dopamine or Dopamine analogs by, for example, ex-vivo treatment of isolated T-cells, and their readministration to the patient, can provide a natural and effective and means of immunosuppression and treatment.

Further, while reducing the present invention to practice, it was uncovered that Dopamine, and Dopamine receptor agonists, can supress T-cell mediated inflammatory and allergic disease. As detailed in Example VII hereinbelow, brief ex vivo exposure of Experimental Autoimmune Encephalitis (EAE)—inducing T-cells (cells sensitized myelin protein) to physiological concentrations of the Dopamine D3 receptor agonist DPAT reduced the severity and fatality of the neurogenic inflammatory condition in recipient mice. Similarly, sensitized T-cells treated ex vivo with physiological concentrations of DPAT were significantly impaired in ability to induce allergic response (Delayed Type Hypersensitivity) in recipient mice. Thus, the method and materials of the present invention can be used to treat or prevent a T-cell inflammatory disease or condition characterized by excessive T-cell activity in a subject by providing a molecule capable of upregulating an activity of a Dopamine receptor. The method further comprises the step of exposing stimulated T-cells from the subject to a therapeutically effective amount of the upregulating molecule. In one preferred embodiment, a symptom of the inflammatory T-cell disease or condition is monitored in the subject prior to and/or following treatment. Suitable assays for determining inflammation and allergic reaction are well known in the art (see, for example, Example VII hereinbelow).

Since EAE is the animal model for Multiple Sclerosis, in one preferred embodiment, the T-cell inflammatory disease is Delayed Type Hypersensitivity, Experimental Autoimmune Encephalomyelitis or Multiple Sclerosis.

Diseases or conditions requiring suppression of immune function may be sensitive to inhibition of T-cell activity by antagonist Dopamine analogs, downregulating anti-Dopamine receptor antibodies, and/or polynucleotides downregulating Dopamine receptor expression. These diseases or conditions include autoimmune states such as systemic lupus erythematosis, rheumatic fever, rheumatoid arthritis, multiple sclerosis Hashimoto's and Grave's disease, Goodpasture's syndrome, myasthenia gravis, insulin-dependent diabetes mellitus, pemphigus vulgaris, Addison's disease, dermatitis herpetiformis and celiac disease; allergic conditions such as atopic dermatitis, allergic asthma, anaphylaxis and other IgE-mediated responses. Similarly, other conditions of undesired T-cell migration and function include T-cell cancer such as T-lymphoma, T-cell mediated graft versus host disease and allograft rejection. Importantly, psychopathological and neurogenic diseases and conditions associated with increased Dopamine-mediated T-cell activity such as schizophrenia, migraine and de novo Parkinson's Disease may be treated with the methods and compounds of the present invention.

While reducing the present invention to practice, it was demonstrated that Dopamine modulation of T-cell function was mediated in part by Dopamine's effect on β-integrin binding to fibronectin, cytokine secretion and T-cell proliferation. Importantly, the β-integrin glycoproteins on the surface of circulating leukocytes recognize and bind to the adhesion proteins expressed on the surface of activated endothelial cells, enabling the migration of leukocytes across the blood vessel walls to the site of the injury or infection. The leukocytes then release chemical mediators, and cytokines to combat the invading matter. In a similar manner, neurogenic diseases such as MS, EAE and meningitis are characterized by indiscriminate destruction of brain tissue caused by the release of toxic mediators by leukocytes which errantly migrate across the blood brain barrier (BBB). Therefore, inhibition of β-integrin binding and T-cell activation by antagonist Dopamine analogs, anti-Dopamine receptor antibodies, and/or polynucleotides downregulating Dopamine receptor expression may be effective in preventing and/or treating T-cell related hyperreactive, autoimmune, allergic, neoplastic, neurogenic, metastatic, psychopathological and infectious conditions.

Thus, according to the present invention there is provided a method of regulating T-cell activity in a mammalian subject having excessive T-cell activity, the method effected by providing to the subject a molecule selected capable of downregulating Dopamine receptor activity or the expression of the gene encoding the Dopamine receptor Similarly, there is provided a method of preventing or treating a T-cell related disease or condition characterized by excessive T-cell activity in a subject having such a disease or condition by providing to the subject a molecule selected capable of downregulating Dopamine receptor activity or the expression of the gene encoding the Dopamine receptor In one embodiment, the downregulator is a D2 or D3 type receptor antagonist such as U-mal or pimozide. In a preferred embodiment, the downregulator is a n anti-Dopamine receptor antibody. In a more preferred embodiment, the downregulator is a single stranded polynucleotide designed having specific Dopamine receptor transcript cleaving capability, an expressible polynucleotide encoding a ribozyme designed having specific Dopamine receptor transcript cleaving capability, a polynucleotide designed comprising nucleotide sequences complementary to, and capable of binding to Dopamine receptor transcripts, coding sequences and/or promoter elements and an expressible polynucleotide encoding nucleotide sequences complementary to, and capable of binding to Dopamine receptor transcripts, coding sequences and/or promoter elements.

As mentioned hereinabove, T-cells may be isolated from the blood by procedures known to one skilled in the art (see, for example, the Materials and Methods section that follows). Thus, in the method of the present invention providing the downregulating molecule is effected by in vivo, by local or systemic administration to the subject via intravenous, parenteral, oral, transdermal, intramuscular, intranasal or other means, or by providing the downregulating molecule to an ex vivo T-cell population, after removal of T-cells from the body and their isolation, and their readministration to the subject, as described in detail hereinabove.

Intracellular levels of Dopamine signal transducers may be manipulated by decreasing the abundance of Dopamine receptor transcripts available for protein synthesis. This may be accomplished by introducing into target cells polynucleotides downregulating Dopamine receptor expression. Delivery of such polynucleotides may be by injection, introduction into the circulation, or introduction into the body cavities by inhalation or insufflation. Thus, according to the present invention, one preferred method of downregulating T-cell activity or an expression of a gene encoding a Dopamine receptor in a mammalian subject is effected by providing to the T-cells of the subject polynucleotides designed having specific Dopamine receptor transcript cleaving or binding capability thereby downregulating Dopamine receptor production, effectively reducing sensitivity to Dopamine activation. The polynucleotides may be ribozymes having specific Dopamine receptor transcript cleaving capabilities, or antisense nucleotide sequences complementary to and capable of reducing Dopamine receptor expression. Similarly, expressible polynucleotides encoding ribozymes or antisense transcripts can be used. These polynucleotide sequences may be introduced into the subject's T-cells and other tissues in vivo or into an ex vivo population of T-cells, by methods of RNA and DNA transfer commonly known in the art such as calcium precipitation, electroporation, microparticle delivery and the like, and readministered to the subject. The preparation and use of such antisense and ribozyme polynucleotides is detailed hereinbelow.

An antisense polynucleotide (e.g., antisense oligodeoxyribonucleotide) may bind its target nucleic acid either by Watson-Crick base pairing or Hoogsteen and anti-Hoogsteen base pairing (Thuong and Helene (1993) Sequence specific recognition and modification of double helical DNA by oligonucleotides Angev. Chem. Int. Ed. Engl. 32:666). According to the Watson-Crick base pairing, heterocyclic bases of the antisense polynucleotide form hydrogen bonds with the heterocyclic bases of target single-stranded nucleic acids (RNA or single-stranded DNA), whereas according to the Hoogsteen base pairing, the heterocyclic bases of the target nucleic acid are double-stranded DNA, wherein a third strand is accommodated in the major groove of the B-form DNA duplex by Hoogsteen and anti-Hoogsteen base pairing to form a triple helix structure.

According to both the Watson-Crick and the Hoogsteen base pairing models, antisense oligonucleotides have the potential to regulate gene expression and to disrupt the essential functions of the nucleic acids in cells. Therefore, antisense polynucleotides have possible uses in modulating a wide range of diseases in which gene expression is altered.

Since the development of effective methods for chemically synthesizing polynucleotides, these molecules have been extensively used in biochemistry and biological research and have the potential use in medicine, since carefully devised polynucleotides can be used to control gene expression by regulating levels of transcription, transcripts and/or translation.

Oligodeoxyribonucleotides as long as 100 base pairs (bp) are routinely synthesized by solid phase methods using commercially available, fully automated synthesis machines. The chemical synthesis of oligoribonucleotides, however, is far less routine. Oligoribonucleotides are also much less stable than oligodeoxyribonucleotides, a fact which has contributed to the more prevalent use of oligodeoxyribonucleotides in medical and biological research, directed at, for example, the regulation of transcription or translation levels.

Gene expression involves few distinct and well regulated steps. The first major step of gene expression involves transcription of a messenger RNA (mRNA) which is an RNA sequence complementary to the antisense (i.e., −) DNA strand, or, in other words, identical in sequence to the DNA sense (i.e., +) strand, composing the gene. In eukaryotes, transcription occurs in the cell nucleus.

The second major step of gene expression involves translation of a protein (e.g., enzymes, structural proteins, secreted proteins, gene expression factors, etc.) in which the mRNA interacts with ribosomal RNA complexes (ribosomes) and amino acid activated transfer RNAs (tRNAs) to direct the synthesis of the protein coded for by the mRNA sequence.

Initiation of transcription requires specific recognition of a promoter DNA sequence located upstream to the coding sequence of a gene by an RNA-synthesizing enzyme—RNA polymerase. This recognition is preceded by sequence-specific binding of one or more transcription factors to the promoter sequence. Additional proteins which bind at or close to the promoter sequence may trans upregulate transcription via cis elements known as enhancer sequences. Other proteins which bind to or close to the promoter, but whose binding prohibits the action of RNA polymerase, are known as repressors.

There are also evidence that in some cases gene expression is downregulated by endogenous antisense RNA repressors that bind a complementary mRNA transcript and thereby prevent its translation into a functional protein.

Thus, gene expression is typically upregulated by transcription factors and enhancers and downregulated by repressors.

However, in many disease situations gene expression is impaired. In many cases, such as different types of cancer, for various reasons the expression of a specific endogenous or exogenous (e.g., of a pathogen such as a virus) gene is upregulated.

The ability of chemically synthesizing oligonucleotides and analogs thereof having a selected predetermined sequence offers means for downmodulating gene expression. Three types of gene expression modulation strategies may be considered.

At the transcription level, antisense or sense oligonucleotides or analogs that bind to the genomic DNA by strand displacement or the formation of a triple helix, may prevent transcription (Thuong and Helene (1993) Sequence specific recognition and modification of double helical DNA by oligonucleotides Angev. Chem. Int. Ed. Engl. 32:666).

At the transcript level, antisense oligonucleotides or analogs that bind target mRNA molecules lead to the enzymatic cleavage of the hybrid by intracellular RNase hours (Dash P., Lotan I., Knapp M., Kandel E. R. and Goelet P. (1987) Selective elimination of mRNAs in vivo: complementary oligodeoxynucleotides promote RNA degradation by an RNase H-like activity. Proc. Natl. Acad. Sci. USA, 84:7896). In this case, by hybridizing to the targeted mRNA, the oligonucleotides or oligonucleotide analogs provide a duplex hybrid recognized and destroyed by the RNase hours enzyme. Alternatively, such hybrid formation may lead to interference with correct is splicing (Chiang M. Y., Chan H., Zounes M. A., Freier S. M., Lima W. F. and Bennett C. F. (1991) Antisense oligonucleotides inhibit intercellular adhesion molecule 1 expression by two distinct mechanisms. J. Biol. Chem. 266:18162-71). As a result, in both cases, the number of the target mRNA intact transcripts ready for translation is reduced or eliminated.

At the translation level, antisense oligonucleotides or analogs that bind target mRNA molecules prevent, by steric hindrance, binding of essential translation factors (ribosomes), to the target mRNA, a phenomenon known in the art as hybridization arrest, disabling the translation of such mRNAs.

Thus, antisense sequences, which as described hereinabove may arrest the expression of any endogenous and/or exogenous gene depending on their specific sequence, attracted much attention by scientists and pharmacologists who were devoted at developing the antisense approach into a new pharmacological tool.

For example, several antisense oligonucleotides have been shown to arrest hematopoietic cell proliferation (Szczylik et al. (1991) Selective inhibition of leukemia cell proliferation by BCR-ABL antisense oligodeoxynucleotides. Science 253:562.), growth (Calabretta et al. (1991) Normal and leukemic hematopoietic cell manifest differential sensitivity to inhibitory effects of c-myc antisense oligodeoxynucleotides: an in vitro study relevant to bone marrow purging. Proc. Nat]. Acad. Sci. USA 88:2351), entry into the S phase of the cell cycle (Heikhila et al. (1987) A c-myc antisense oligodeoxynucleotide inhibits entry into S phase but not progress from G(0) to G(l). Nature, 328:445), reduced survival (Reed et al. (1990) Antisense mediated inhibition of BCL2 prooncogene expression and leukemic cell growth and survival: comparison of phosphodiester and phosphorothioate oligodeoxynucleotides. Cancer Res. 50:6565), prevent receptor mediated responses (Burch and Mahan (1991) Oligodeoxynucleotides antisense to the interleukin I receptor m RNA block the effects of interleukin I in cultured murine and human fibroblasts and in mice. J. Clin. Invest. 88:1190) and as antiviral agents (Agrawal (1992) Antisense oligonucleotides as antiviral agents. TIBTECH 10: 152).

For efficient in vivo inhibition of gene expression using antisense oligonucleotides or analogs, the oligonucleotides or analogs must fulfill the following requirements (i) sufficient specificity in binding to the target sequence; (ii) solubility in water; (iii) stability against intra- and extracellular nucleases; (iv) capability of penetration through the cell membrane; and (v) when used to treat an organism, low toxicity.

Unmodified oligonucleotides are impractical for use as antisense sequences since they have short in vivo half-lives, during which they are degraded rapidly by nucleases. Furthermore, they are difficult to prepare in more than milligram quantities. In addition, such oligonucleotides are poor cell membrane penetrators.

Thus it is apparent that in order to meet all the above listed requirements, oligonucleotide analogs need to be devised in a suitable manner. Therefore, an extensive search for modified oligonucleotides has been initiated.

For example, problems arising in connection with double-stranded DNA (dsDNA) recognition through triple helix formation have been diminished by a clever “switch back” chemical linking, whereby a sequence of polypurine on one strand is recognized, and by “switching back”, a homopurine sequence on the other strand can be recognized. Also, good helix formation has been obtained by using artificial bases, thereby improving binding conditions with regard to ionic strength and pH.

In addition, in order to improve half-life as well as membrane penetration, a large number of variations in polynucleotide backbones have been done, nevertheless with little success.

Oligonucleotides can be modified either in the base, the sugar or the phosphate moiety. These modifications include, for example, the use of is methylphosphonates, monothiophosphates, dithiophosphates, phosphoramidates, phosphate esters, bridged phosphorothioates, bridged phosphoramidates, bridged methylenephosphonates, dephospho intemucleotide analogs with siloxane bridges, carbonate bridges, carboxymethyl ester bridges, carbonate bridges, carboxymethyl ester bridges, acetamide bridges, carbamate bridges, thioether bridges, sulfoxy bridges, sulfono bridges, various “plastic” DNAs, Ct-anomeric bridges and borane derivatives. For illustrative examples and further details see Cook (1991) Medicinal chemistry of antisense oligonucleotides—future opportunities. Anti-Cancer Drug Design 6:585.

International patent application WO 89/12060 discloses various building blocks for synthesizing oligonucleotide analogs, as well as oligonucleotide analogs formed by joining such building blocks in a defined sequence. The building blocks may be either “rigid” (i.e., containing a ring structure) or “flexible” (i.e., lacking a ring structure). In both cases, the building blocks contain a hydroxy group and a mercapto group, through which the building blocks are said to join to form oligonucleotide analogs. The linking moiety in the oligonucleotide analogs is selected from the group consisting of sulfide (—S—), sulfoxide (—SO—), and sulfone (—SO₂—). However, the application provides no data supporting the specific binding of an oligonucleotide analog to a target oligonucleotide.

International patent application WO 92/20702 describe an acyclic oligonucleotide which includes a peptide backbone on which any selected chemical nucleobases or analogs are stringed and serve as coding characters as they do in natural DNA or RNA. These new compounds, known as peptide nucleic acids (PNAs), are not only more stable in cells than their natural counterparts, but also bind natural DNA and RNA 50 to 100 times more tightly than the natural nucleic acids cling to each other. PNA oligomers can be synthesized from the four protected monomers containing thymine, cytosine, adenine and guanine by Merrifield solid-phase peptide synthesis. In order to increase solubility in water and to prevent aggregation, a lysine amide group is placed at the C-terminal.

Thus, antisense technology requires pairing of messenger RNA with an oligonucleotide to form a double helix that inhibits translation. The concept of antisense-mediated gene therapy was already introduced in 1978 for cancer therapy. This approach was based on certain genes that are crucial in cell division and growth of cancer cells. Synthetic fragments of genetic substance DNA can achieve this goal. Such molecules bind to the targeted gene molecules in RNA of tumor cells, thereby inhibiting the translation of the genes and resulting in dysfunctional growth of these cells. Other mechanisms has also been proposed. These strategies have been used, with some success in treatment of cancers, as well as other illnesses, including viral and other infectious diseases. Antisense polynucleotides are typically synthesized in lengths of 13-30 nucleotides. The life span of oligonucleotide molecules in blood is rather short. Thus, they have to be chemically modified to prevent destruction by ubiquitous nucleases present in the body. Phosphorothioates are very widely used modification in antisense oligonucleotide ongoing clinical trials. A new generation of antisense molecules consist of hybrid -antisense oligonucleotide with a central portion of synthetic DNA while four bases on each end have been modified with 2′O-methyl ribose to resemble RNA. In preclinical studies in laboratory animals, such compounds have demonstrated greater stability to metabolism in body tissues and an improved safety profile when compared with the first-generation unmodified phosphorothioate (Hybridon Inc. news). Dozens of other nucleotide analogs have also been tested in antisense technology.

RNA oligonucleotides may also be used for antisense inhibition as they form a stable RNA-RNA duplex with the target, suggesting efficient inhibition. However, due to their low stability RNA oligonucleotides are typically expressed inside the cells using vectors designed for this purpose. This approach is favored when attempting to target a mRNA that encodes an abundant and long-lived protein.

Recent scientific publications have validated the efficacy of antisense compounds in animal models of hepatitis, cancers, coronary artery restenosis and other diseases. The first antisense drug was recently approved by the FDA. This drug Fomivirsen, developed by Isis, is indicated for local treatment of cytomegalovirus in patients with AIDS who are intolerant of or have a contraindication to other treatments for CMV retinitis or who were insufficiently responsive to previous treatments for CMV retinitis (Pharmacotherapy News Network).

Several antisense compounds are now in clinical trials in the United States. These include locally administered, antivirals, systemic cancer therapeutics. Antisense therapeutics has the potential to treat many life-threatening diseases with a number of advantages over traditional drugs. Traditional drugs intervene after a disease-causing protein is formed. Antisense therapeutics, however, block mRNA transcription/translation and intervene before a protein is formed, and since antisense therapeutics target only one specific mRNA, they should be more effective with fewer side effects than current protein-inhibiting therapy.

Antisense therapy has also been applied to immune disorders and inhibition of cell migration. For example, U.S. Pat. No. 6,096,722 to Bennet et al. discloses the application of antisense polynucleotides to interrupt cell adhesion molecules (CAM) expression in the treatment of pathogenic, autoimmune, allergic, chronic inflammatory, hyperproliferation and metastatic conditions. International Application No. WO 97/39721 to Glimcher et al discloses the use of antisense polynucleotides to T-cell activation and cytokine expression.

A second option for disrupting gene expression at the level of transcription uses synthetic oligonucleotides capable of hybridizing with double stranded DNA. A triple helix is formed. Such oligonucleotides may prevent binding of transcription factors to the gene's promoter and therefore inhibit transcription. Alternatively, they may prevent duplex unwinding and, therefore, transcription of genes within the triple helical structure.

Another approach is the use of specific nucleic acid sequences to act as decoys for transcription factors. Since transcription factors bind specific DNA sequences it is possible to synthesize oligonucleotides that will effectively compete with the native DNA sequences for available transcription factors in vivo. This approach requires the identification of gene specific transcription factor.

Indirect inhibition of gene expression was demonstrated for matrix metalloproteinase genes (MMP-1, -3, and -9), which are associated with invasive potential of human cancer cells. EIAF is a transcription activator of MMP genes. Expression of EIAF antisense RNA in HSC3AS cells showed decrease in mRNA and protein levels of MMP-1, -3, and -9. Moreover, HSC3AS showed lower invasive potential in vitro and in vivo. These results imply that transfection of antisense inhibits tumor invasion by down-regulating MMP genes.

Ribozymes are being increasingly used for the sequence-specific inhibition of gene expression by the cleavage of mRNAs encoding proteins of interest. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated-WEB home page).

As used herein, “ribozymes” are intended to include RNA molecules that contain anti-sense sequences for specific recognition, and an RNA-cleaving enzymatic activity. The catalytic strand cleaves a specific site in a target RNA at greater than stoichiometric concentration. Two “types” of ribozymes are particularly useful in this invention, the hammerhead ribozyme (Rossi, J. J. et al., Pharmac. Ther. 50:245-254, 1991) and the hairpin ribozyme (Hampel et al., Nucl. Acids Res. 18:299-304, 1990, and U.S. Pat. No. 5,254,678, issued Oct. 19, 1993). Because both hammerhead and hairpin ribozymes are catalytic molecules having antisense and endoribonucleotidase activity, ribozyme technology has emerged as a potentially powerful extension of the antisense approach to gene inactivation. The ribozymes of the invention typically consist of RNA, but such ribozymes may also be composed of nucleic acid molecules comprising chimeric nucleic acid sequences (such as DNA/RNA sequences) and/or nucleic acid analogs (e.g., phosphorothioates).

Ribozymes may be in the form of a “hammerhead” (for example, as described by Forster and Symons, Cell 48:211-220, 1987; Haseloff and Gerlach, Nature 328:596-600, 1988; Walbot and Bruening, Nature 334:196, 1988; Haseloff and Gerlach, Nature 334:585, 1988) or a “hairpin” (for example, as described by Haseloffet al., U.S. Pat. No. 5,254,678, issued Oct. 19, 1993 and Hempel et al., European Patent Publication No. 0 360 257, published Mar. 26, 1990). The sequence requirement for the hairpin ribozyme is any RNA sequence consisting of NNNBN*GUC (where N*G is the cleavage site, where B is any of G, C, or U, and where N is any of G, U, C, or A)(SEQ ID NO: 9). The sequence requirement at the cleavage site for the hammerhead ribozyme is any RNA sequence consisting of NUX (where N is any of G, U, C, or A and X represents C,. U, or A) can be targeted. Accordingly, the same target within the hairpin leader sequence, GUC, is useful for the hammerhead ribozyme. The additional nucleotides of the hammerhead ribozyme or hairpin ribozyme is determined by the target flanking nucleotides and the hammerhead consensus sequence (see Ruffner et al., Biochemistry 29:10695-10702, 1990).

This information, and the published sequence of mRNA coding sequences for human Dopamine receptors D4 (Genbank accession number XM 006145; NCBI Annotation Project)(SEQ ID NO:10), D1(Genbank accession number XM 003966; NCBI Annotation Project)(SEQ ID NO: 11), D5 (Genbank accession number BC009748; Strausberg, R.)(SEQ ID NO 12) D3 (Genbank accession number HSU 32499; Fishburn C S et al)(SEQ ID NO: 13), and the genomic DNA sequence coding for human Dopamine receptor D2 (Genbank accession number AF050737; Hauge, X Y et al)(SEQ ID NO:14), enables the production of the ribozymes of this invention. Appropriate base changes in the ribozyme is made to maintain the necessary base pairing with the target RNA sequences.

Cech et al. (U.S. Pat. No. 4,987,071) has disclosed the preparation and use of certain synthetic ribozymes which have endoribonuclease activity. These ribozymes are based on the properties of the Tetrahymena ribosomal RNA self-splicing reaction and require an eight base pair target site. The ribozymes of this invention, as well as DNA encoding such ribozymes and other suitable nucleic acid molecules, can be chemically synthesized using methods well known in the art for the synthesis of nucleic acid molecules. Alternatively, Promega, Madison, Wis., USA, provides a series of protocols suitable for the production of RNA molecules such as ribozymes. The ribozymes also can be prepared from a DNA molecule or other nucleic acid molecule (which, upon transcription, yields an RNA molecule) operably linked to an RNA polymerase promoter, e.g., the promoter for T7 RNA polymerase or SP6 RNA polymerase. Such a construct may be referred to as a vector. Accordingly, also provided by this invention are nucleic acid molecules, e.g., DNA or cDNA, coding for the ribozymes of this invention. When the vector also contains an RNA polymerase promoter operably linked to the DNA molecule, the ribozyme can be produced in vitro upon incubation with the RNA polymerase and appropriate nucleotides. Alternatively, the DNA may be inserted into an expression cassette, such as described in Cotten and Birnstiel, EMBO J 8(12):3861-3866, 1989, and in Hempel et al., Biochemistry 28:4929-4933, 1989. A more detailed discussion of molecular biology methodology is disclosed in Sambrook et al., Molecular Cloning: A Laboratory Manual, Cold Spring Harbor Press, 1989.

After synthesis, the ribozyme can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

In one preferred embodiment of the present invention, the expressible downregulating polynucleotide is designed capable of transient expression in cells of the subject. In another preferred embodiment, the expressible polynucleotide is designed capable of stably integrating into the genome of cells of the subject.

Thus, the ribozyme molecule also can be in a host procaryotic or eukaryotic cell in culture or in the cells of an organism. Appropriate prokaryotic and eukaryotic cells can be transfected with an appropriate transfer vector containing the DNA molecule encoding a ribozyme of this invention. Alternatively, the ribozyme molecule, including nucleic acid molecules encoding the ribozyme, may be introduced into the host cell using traditional methods such as transformation using calcium phosphate precipitation (Dubensky et al., PNAS 81:7529-7533, 1984), direct microinjection of such nucleic acid molecules into intact target cells (Acsadi et al., Nature 352:815-818, 1991), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al., PNAS 89:6094, 1990), lipofection (Felgner et al., Proc. Natl. Acad. Sci. USA 84:7413-7417, 1989), microprojectile bombardment (Williams et al., PNAS 88:2726-2730, 1991), polycation compounds such as polylysine, receptor specific ligands, liposomes entrapping the nucleic acid molecules, spheroplast fusion whereby E coli containing the nucleic acid molecules are stripped of their outer cell walls and fused to animal cells using polyethylene glycol, viral transduction, (Cline et al., Pharmac. Ther. 29:69, 1985; and Friedmann et al., Science 244:1275, 1989), and DNA ligand (Wu et al. J. of Biol Chem. 264:16985-16987, 1989), as well as psoralen inactivated viruses such as Sendai or Adenovirus. In a preferred embodiment, the ribozyme is introduced into the host cell utilizing a liposome.

When the DNA molecule is operatively linked to a promoter for RNA transcription, the RNA can be produced in the host cell when the host cell is grown under suitable conditions favoring transcription of the DNA molecule. The vector can be, but is not limited to a plasmid, a virus, a retrotransposon or a cosmid. Examples of such vectors are disclosed in U.S. Pat. No. 5,166,320. Other representative vectors include adenoviral vectors (e.g., WO 94/26914, WO 93/9191; Kolls et al., PNAS 91(1):215-219, 1994; Kass-Eisler et al., PNAS 90(24):11498-502, 1993; Guzman et al., Circulation 88(6):2838-48, 1993; Guzman et al., Cir. Res. 73(6):1202-1207, 1993; Zabner et al., Cell 75(2):207-216, 1993; Li et al., Huim Gene Ther. 4(4):403-409, 1993; Caillaud et al., Eur. J Neurosci. 5(10):1287-1291, 1993), adeno-associated vector type 1 (“AAV-1”) or adeno-associated vector type 2 (“AAV-2”) (see WO 95/13365; Flotte et al., PNAS 90(22):10613-10617, 1993), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick, J. W. and Burck, K. L., Gene Therapy: Application of Molecular Biology, Elsevier Science Publishing Co., Inc., New York, N.Y., 1991 and Kreigler, M., Gene Transfer and Expression: A Laboratory Manual, W.H. Freeman and Company, New York, 1990. To produce ribozymes in vivo utilizing vectors, the nucleotide sequences coding for ribozymes are preferably placed under the control of a strong promoter such as the lac, SV40 late, SV40 early, or lambda promoters. Ribozymes are then produced directly from the transfer vector in vivo.

Observations in the early 1990s that plasmid DNA could directly transfect animal cells in vivo sparked exploration of the use of DNA plasmids to induce immune response by direct injection into animal of DNA encoding antigenic protein. When a DNA vaccine plasmid enters the eukaryotic cell, the protein it encodes is transcribed and translated within the cell. In the case of pathogens, these proteins are presented to the immune system in their native form, mimicking the presentation of antigens during a natural infection. DNA -vaccination is particularly useful for the induction of T cell activation. It was applied for viral and bacterial infectious diseases, as well as for allergy and for cancer. The central hypothesis behind active specific immunotherapy for cancer is that tumor cells express unique antigens that should stimulate the immune system. The first DNA vaccine against tumor was carcino-embrionic antigen (CEA). DNA vaccinated animals expressed immunoprotection and immunotherapy of human CEA-expressing syngeneic mouse colon and breast carcinoma. In a mouse model of neuroblastoma, DNA immunization with HuD resulted in tumor growth inhibition with no neurological disease. Immunity to the brown locus protein, gp⁷⁵ tyrosinase-related protein-1, associated with melanoma, was investigated in a syngeneic mouse model. Priming with human gp75 DNA broke tolerance to mouse gp75. Immunity against mouse gp75 provided significant tumor protection.

The present invention has the potential to provide transgenic gene and polymorphic gene animal and cellular (cell lines) models as well as for knockout models. These models may be constructed using standard methods known in the art and as set forth in U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270 1991); Capecchi, Science 244:1288-1292 1989); Davies et al., Nucleic Acids Research, 20 (11) 2693-2698 1992); Dickinson et al., Human Molecular Genetics, 2( 8): 1299-1302 1993); Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991); Jakobovits et al., Nature, 362:255-261 1993); Lamb et al., Nature Genetics, 5: 22-29 1993); Pearson and Choi, Proc. Natl. Acad. Sci. USA 1993). 90:10578-82; Rothstein, Methods in Enzymology, 194:281-301 1991); Schedi et al., Nature, 362: 258-261 1993);

Strauss et al., Science, 259:1904-1907 1993). Further, patent applications WO 94/23049, WO 93/14200, WO 94/06908, WO 94/28123 also provide information.

Gene therapy as used herein refers to the transfer of genetic material (e.g. DNA or RNA) of interest into a host to treat or prevent a genetic or acquired disease or condition or phenotype. The genetic material of interest encodes a product (e.g. a protein, polypeptide, peptide, functional RNA, antisense) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject rather the genetic material to be transferred is introduced into the cells of the recipient organism in situ, that is within the recipient. In an alternative embodiment, if the host gene is defective, the gene is repaired in situ (Culver, 1998. (Abstract) Antisense DNA & RNA based therapeutics, February 1998, Coronado, Calif.). These genetically altered cells have been shown to express the transfected genetic material in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore, as used herein the expression vehicle may, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any nontranslated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York 1989, 1992), in Ausubel et al., Current Protocols in Molecular Biology, John Wiley and Sons, Baltimore, Md. 1989), Chang et al., Somatic Gene Therapy, CRC Press, Ann Arbor, Mich. 1995), Vega et al., Gene Targeting, CRC Press, Ann Arbor Mich. (995), Vectors: A Survey of Molecular Cloning Vectors and Their Uses, Butterworths, Boston Mass. 1988) and Gilboa et al. (Biotechniques 4 (6): 504-512, 1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector introducing and expressing recombination sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in may cases, to elude host defense mechanisms. Typically, viruses infect and propagate in specific cell types. The targeting specificity of viral utilizes its natural specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration, especially in the case of neuro-degenerative diseases. Following injection, the viral vectors will circulate until they recognize host cells with appropriate target specificity for infection.

Antisense, ribozyme and DNA therapy may be targeted to the Dopamine receptor, effectively reducing the ability of the treated T-cells to respond to stimulation by Dopamine, or Dopamine agonistic analogs. For example, Baserga et al. (U.S. Pat. No. 6,274,562) discloses the application of antisense constructs against IGF-I receptor transcripts to inhibit proliferation and cause differentiation of the IGF-I sensitive cells. Schreiber et al. (U.S. Pat. No. 6,242,427) disclose antisense constructs for treatment of inflammatory conditions by inhibiting Fc receptor expression in phagocytic cells. Similarly, U.S. Pat. No. 5,622,854 to Draper discloses, in detail, methods for the transformation of T-cells with expressible polynucleotides.

The molecules of the present invention can also include small interfering duplex oligonucleotides [i.e., small interfering RNA (siRNA)], which direct sequence specific degradation of mRNA through the previously described mechanism of RNA interference (RNAi) [Hutvagner and Zamore (2002) Curr. Opin. Genetics and Development 12:225-232].

As used herein, the phrase “duplex oligonucleotide” refers to an oligonucleotide structure or mimetics thereof, which is formed by either a single self-complementary nucleic acid strand or by at least two complementary nucleic acid strands. The “duplex oligonucleotide” of the present invention can be composed of double-stranded RNA (dsRNA), a DNA-RNA hybrid, single-stranded RNA (ssRNA), isolated RNA (i.e., partially purified RNA, essentially pure RNA), synthetic RNA and recombinantly produced RNA.

Instructions for generation of duplex oligonucleotides capable of mediating RNA interference are provided in www.ambion.com.

In one preferred embodiment, the ribozyme, antisense or siRNA polynucleotides are directed against Dopamine D1, D2, D3, D4 or D5 receptors. Thus, the downregulating expressible polynucleotides may include a sequence as set forth in any of SEQ ID NOs: 10-14.

In a preferred embodiment, downregulation of T-cell activity by ribozyme, antisense or DNA methodology directed against the Dopamine receptor is applied where the mammalian subject is suffering from excessive T-cell activity such as in autoimmune, neoplastic, hyperreactive, psychopathological and neurogenic and allergic diseases and conditions; graft versus host disease and allograft rejection.

The methods and materials of the present invention may be used in the treatment of subjects suffering from cancerous disease or conditions. Patients having hyperproliferative disorders, which include both benign tumors and primary malignant tumors that have been detected early in the course of their development, may often be successfully treated by the surgical removal of the benign or primary tumor. If unchecked, however, cells from malignant tumors are spread throughout a patient's body through the processes of invasion and metastasis. Invasion refers to the ability of cancer cells to detach from a primary site of attachment and penetrate, e.g., an underlying basement membrane. Metastasis indicates a sequence of events wherein (1) a cancer cell detaches from its extracellular matrices, (2) the detached cancer cell migrates to another portion of the patient's body, often via the circulatory system, and (3) attaches to a distal and inappropriate extracellular matrix, thereby created a focus from which a secondary tumor can arise. Normal cells do not possess the ability to invade or metastasize and/or undergo apoptosis (programmed cell death) if such events occur (Ruoslahti, Sci. Amer., 1996, 275, 72).

Disseminating precancerous or cancerous cells often display ectopic expression of substrate binding molecules which may facilitate step (3) of the metastatic process as described above. Thus, modulation of β-integrin binding using the antisense compounds of the invention may result in a decreased ability of disseminating T-cell related cancer cells to migrate. The importance of ECM binding proteins to extravasation and metastatic spread of T-lymphoma and other cancer cells has been noted (see, for example, Wewer, U. M. et al. , Proc Natl Acad Sci USA 1986; 83: 7137-41, and Hand, P. H. et al. Cancer Research 1985; 45: 2713-19).

While reducing the present invention to practice, it was noted that Dopamine stimulated β-integrin binding in human T-cells. Thus, inhibition of sensitivity to Dopamine stimulation may be effective in downregulating β-integrin binding, providing a novel therapeutic approach for the treatment of T-cell related cancers. Thus, according to a further aspect of the present invention there is provided a method of treating or preventing a cancerous disease or condition in a subject suffering from a cancerous disease or condition characterized by excess T-cell activity, by providing to the subject a therapeutically effective amount of a molecule selected capable of downregulating an activity of a Dopamine receptor or an expression of a gene encoding a Dopamine receptor. The method further comprising the step of determining the cancer cell proliferation and/or metastasis in the subject prior to, and/or following the treatment. In preferred embodiments of the present invention the downregulating molecules are anti-Dopamine antibodies, Dopamine antagonists, and downregulating polynucleotides such as antisense, ribozyme and/or expressible polynucleotides encoding antisense or ribozyme oligoneucleotides capable of effectively reducing Dopamine receptor transcripts, as described above, and may be introduced to the subject by systemic or local administration in vivo, or to an ex vivo population of the subject's T-cells, and readministered, as detailed hereinabove. In another preferred embodiment, the cancerous disease or condition is a myeloproliferative disease, such as Leukemia or T-cell cancer. Treatment of the T-cell cancer cells may be in combination with one or more additional anticancer compounds and/or chemotherapeutic drugs. The downregulating molecules of the invention are evaluated for their ability to modulate proliferation and/or metastasis using one or more assays known in the art and/or one or more appropriate animal models (see, for example, Johnston, J A et al, 1994 J. Immunol 153, 1762-68).

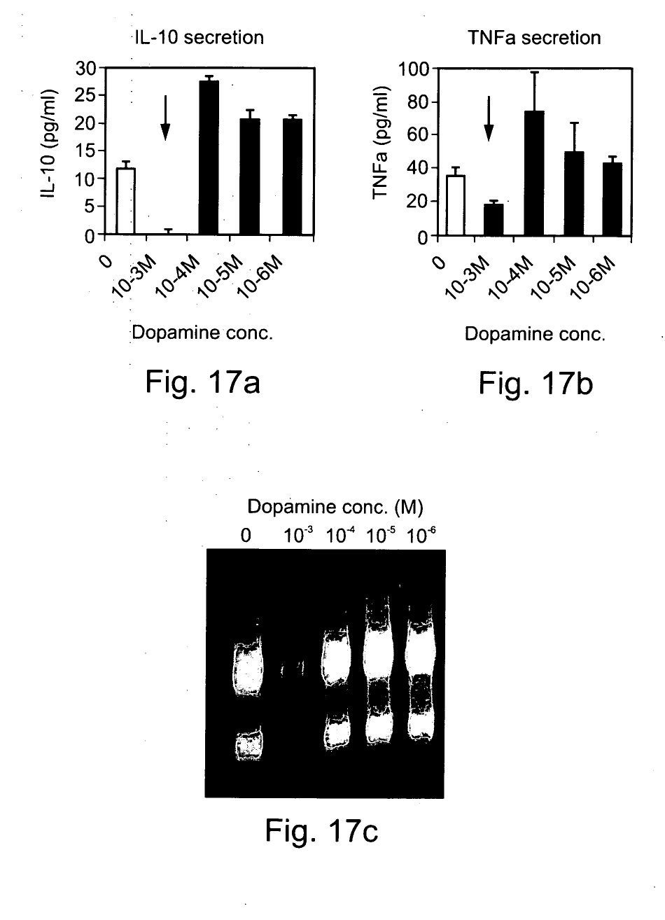

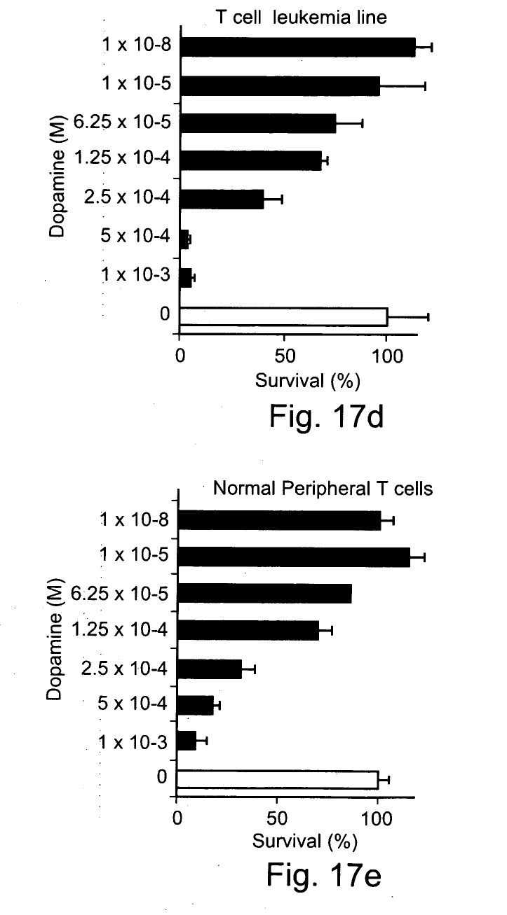

Hyperreactive, hyperproliferative and cancerous T-cells may be suppressed by methods of the present invention. While reducing to practice, it was unexpectedly uncovered that high, unphysiological concentrations of Dopamine and Dopamine upregulating analogs inhibited T-cell function, as measured by cytokine secretion and proliferation (see Example X and FIG. 17 hereinbelow). At higher concentrations, T-cell apoptosis was induced. Measuring specific cell survival rate at a range of Dopamine concentrations, it became clear that Dopamine concentrations in the range of 2.5×10⁻⁴M to 10⁻³M dramatically impaired both normal human and Jurkat leukemia T-cell survival. Thus, according to a further aspect of the present invention, there is provided a method of suppressing activity of a T-cell population. The method is effected by exposing the T-cell population with a concentration of a molecule selected capable of upregulating a Dopamine receptor activity, said concentration sufficient to suppress T-cell function in the T-cell population. In one preferred embodiment, the molecule selected capable of upregulating a Dopamine receptor activity is Dopamine or a Dopamine analog, in a concentration greater than 10⁻⁴ M. Such T-cell suppression can be used to inhibit and eliminate populations of leukemic or hyperreactive T-cells, without the additional toxicity of conventional metabolic suppression. Furthermore, the Dopamine induced T-cell apoptosis, demonstrated here for the first time, is a significant consideration in considering the conventional treatment of severe heart failure, in which high doses of Dopamine (5-20 μg/kg/minute) are often infused to improve cardiac contractility and output.

Further according to the present invention there is provided an assay for determining the sensitivity of a resting T-cell population to regulation of Dopamine receptor activity. The assay is effected by exposing the T-cell population to a molecule selected capable of regulating a Dopamine receptor activity or the expression of a gene encoding a Dopamine receptor, and assessing the state of the T-cell population.

In one preferred embodiment, the assay is performed by exposing the T-cell population to a range of concentrations of the Dopamine receptor regulator, and assessing the state of the T-cell population at each concentration of the range, as described in detail in Example X, FIG. 17. Specific examples of such assays, using molecules capable of upregulating and downregulating T-cell Dopamine receptor activity, are detailed throughout the Examples section hereinbelow (see, for example, Examples I-V). As described therein, T-cell functions such as fibronectin adhesion, cytokine secretion, proliferation, up-and downregulation of specific genes and membrane depolarization can be assayed to determine the sensitivity of Dopamine receptor regulators. Likewise, the effect of the abovementioned upregulating modulators may be assayed in a T-cell population isolated from a subject suffering from an immune deficiency, infectious, age-related, neurogenic, psychopathological or other disease or condition requiring enhanced T-cell activity (see abovementioned list of conditions).

Similarly, efficacy, potency and receptor specificity of putative Dopamine receptor regulators may be determined using the assay of the present invention. Changes in a designated state of test T-cell populations can be compared with changes in populations exposed to known, reference regulators. Such an assay can also be used to characterize and compare individual T-cell populations, such as T-cell leukemic cells and T-cell lines.

In a further embodiment, the molecule is an expressible polynucleotide designed capable of regulating expression of a gene encoding a Dopamine receptor. The expressible polynucleotides may be designed capable of transient expression within the cells of the T-cell population, or designed capable of stably integrating into the genome of cells of the T-cell population expression in the T-cell.

In the case of a T cell related neoplastic disease, the assay may be effected by exposing a T-cell related cancer cell to one or more concentrations of a Dopamine analog and assessing the ability of the cancer cell to proliferate and/or metastasize. In a preferred embodiment the Dopamine analog concentration may be 0.1 ng/ml to 1 mg/ml, sufficient to produce a significant alteration in activation, as measured by, for example, fibronectin binding, radiolabeled precursor uptake, mitotic index, specific gene expression and the like (see Examples section that follows). The assay may be performed in vitro or in vivo, using T-cell related cancer cells. By varying the assay conditions, the sensitivity of a cancer cell to Dopamine analog inhibition of proliferation and metastasis may be assessed. The Dopamine analog may a naturally occurring or synthetic analog.

As used herein, the term “Dopamine analog” refers to a modified amino acid or other molecule or molecules having stimulatory (agonist) or inhibitory (antagonist) action on one or more Dopamine-mediated target cell function. Thus, Dopamine analogs may specifically bind D1 and/or D2 -like Dopamine receptors, blocking or, alternately stimulating characteristic Dopamine signal transduction pathways. Many such analogs are commercially available to one skilled in the art (see, for example, the list of Dopaminergics provided by Research Biochemicals Incorporated, Nattick, Mass., USA). In one preferred embodiment, agonist analogs were 7-OH-DPAT, bromocryptine and pergolide (Sigma Chemicals, St. Louis Mo.), and antagonist analogs were U-99194A maleate (U-Mal), butaclamol, haloperidol and pimozide (Sigma Chemicals, St.Louis Mo.).

As used herein, the term “naturally occurring” as applied to an object refers to the fact that the object may be found in nature. For example, a catecholamine or polynucleotide sequence that is present in an organism (including viruses) that can be isolated from a source in nature and which has not been intentionally modified by man in the laboratory is naturally-occurring.

As used herein, the term “amino acid” or “amino acids” is understood to include the 20 naturally occurring amino acids; those amino acids modified in vivo, including hydroxyproline, phosphoserine and phosphothreonine; and other unusual amino acids including, for example 2-aminoadipic acid, hydroxylysine, isodesmosine, nor-valine, nor-leucine and ornithine. The term “amino acid” includes both D- and L-amino acids.

Similarly, the assay of the present invention may be applied to additional methods of upregulating T-cell activity. Thus, the sensitivity of a T-cell to upregulating analogs, or to expressible polynucleotides encoding Dopamine receptors and/or to upregulating anti-Dopamine receptor antibodies may be assayed. Exposure of the T-cells to the upregulating modulators may be performed in vivo, in vitro or ex vivo, as described in the Examples section that follows.

Further according to the present invention, there is provided an article of manufacture comprising packaging material and a therapeutically effective amount of a pharmaceutical composition identified for treatment of a T-cell related disease or condition associated with abnormal T-cell activity, the pharmaceutical composition including a molecule selected capable of regulating an activity of a Dopamine receptor or an expression of a gene encoding the Dopamine receptor in T cells, and a pharmaceutically effective carrier. The pharmaceutical composition is identified as effective for treatment of the T-cell related disease or condition by a label or insert included in the packaging material, bearing, for example, clinical indications for use, notification of FDA approval, recommended dosages, frequency and modes of administration, contraindications and the like.

In one preferred embodiment, the pharmaceutical composition comprising as an active ingredient a molecule selected capable of upregulating Dopamine receptor activity, or the expression of a gene encoding the Dopamine receptor, packaged and identified for use in the prevention and/or treatment of a T cell related disease or condition characterized by suboptimal T-cell activity. The Dopamine receptor upregulator can be Dopamine, an upregulating Dopamine analog such as DPAT, an upregulating anti-Dopamine receptor antibody or an expressible polynucleotide encoding a Dopamine receptor.

In another embodiment, the pharmaceutical composition comprises a downregulator of Dopamine receptor activity, as described in detail hereinabove. Such an article of manufacture comprising the downregulating pharmaceutical composition, packaged and identified for use to treat or prevent a T-cell related disease or condition characterized by excessive T-cell activity, as described in detail hereinabove.

The compositions of the present invention include bioequivalent compounds, including pharmaceutically acceptable salts and prodrugs. This is intended to encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to pharmaceutically acceptable salts of the nucleic acids of the invention and prodrugs of such nucleic acids. “Pharmaceutically acceptable salts” are physiologically and pharmaceutically acceptable salts of the nucleic acids of the invention: i.e., salts that retain the desired biological activity of the parent compound and do not impart undesired toxicological effects thereto (see, for example, Berge et al., “Pharmaceutical Salts,” J. of Pharma Sci. 1977, 66, 1-19).

For therapeutic or prophylactic treatment, peptides, peptide fragments, polynucleotides and antibodies are administered in accordance with this invention. Components of the invention may be formulated in a pharmaceutical composition, which may include pharmaceutically acceptable carriers, thickeners, diluents, buffers, preservatives, surface active agents, neutral or cationic lipids, lipid complexes, liposomes, penetration enhancers, carrier compounds and other pharmaceutically acceptable carriers or excipients and the like in addition to the peptides, peptide fragments, polynucleotides and antibodies. Such compositions and formulations are comprehended by the present invention.

As used herein, the term “pharmaceutically acceptable carrier” (excipient) indicates a pharmaceutically acceptable solvent, suspending agent or any other pharmacologically inert vehicle for delivering one or more nucleic acids to an animal. The pharmaceutically acceptable carrier may be liquid or solid and is selected with the planned manner of administration in mind so as to provide for the desired bulk, consistency, etc., when combined with a nucleic acid and the other components of a given pharmaceutical composition. Typical pharmaceutically acceptable carriers include, but are not limited to, binding agents (e.g., pregelatinized maize starch, polyvinyl-pyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.). Sustained release oral delivery systems and/or enteric coatings for orally administered dosage forms are described in U.S. Pat. Nos. 4,704,295; 4,556,552; 4,309,406; and 4,309,404.

The compositions of the present invention may additionally contain other adjunct components conventionally found in pharmaceutical compositions, at their art-established usage levels. Thus, for example, the compositions may contain additional compatible pharmaceutically-active materials such as, e.g., antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the invention.

Regardless of the method by which the Dopamine analogs, polynucleotides and antibodies of the invention are introduced into a patient, colloidal dispersion systems may be used as delivery vehicles to enhance the in vivo stability of the and/or to target the analogs, polynucleotides and antibodies to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:catecholamine, polynucleotide and/or antibody complexes of uncharacterized structure. A preferred colloidal dispersion system is a plurality of liposomes. Liposomes are microscopic spheres having an aqueous core surrounded by one or more outer layers made up of lipids arranged in a bilayer configuration (see, generally, Chonn et al., Current Op. Biotech. 1995, 6, 698-708).

For therapeutic uses, the pharmaceutical compositions of the present invention may be administered in a number of ways depending upon whether local or systemic treatment is desired and upon the area to be treated. Administration may be topical (including ophthalmic and to mucous membranes including vaginal and rectal delivery) pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

For certain conditions, particularly skin conditions including but not limited to, psoriasis, administration of compounds to the skin is preferred. Administration of compounds to the skin may be done in several ways including topically and transdermally. A preferred method for the delivery of biologically active substances to the skin is topical administration. “Topical administration” refers to the contacting, directly or otherwise, to all or a -portion of the skin of an animal. Compositions for topical administration may be a mixture of components or phases as are present in emulsions (including microemulsions and creams), and related formulations comprising two or more phases. Transdermal drug delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of penetration enhancers. Hydration of the skin and the use of controlled release topical patches are also effective ways to deliver drugs via the transdermal route. This route provides an effective means to deliver drugs for both systemic and local therapy.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991,p. 166), and optimization of vehicle characteristics relative to dose:deposition and retention at the site of administration (Lee et al., Critical Reviews in Therapeutic Drug Carrier Systems, 1991, p. 168) may be useful methods for enhancing the transport of drugs across mucosal sites in accordance with the present invention.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiugi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if Filly set forth herein. Other general references are provided throughout this document. The procedures therein are believed to be well known in the art and are provided for the convenience of the reader. All the information contained therein is incorporated herein by reference.

### Materials and Experimental Methods

Materials: The following were obtained from the sources indicated: Dopamine receptor agonists and antagonists: 7-OH-DPAT, U-99 1 94A Maleate (U-Mal), Pimozide, Bromocryptine, Pergolide, Haloperidol and Butaclamol, fibronectin, Bovine Serum Albumin (BSA), gly-arg-gly-asp-ser (GRGDS)(SEQ ID NO: 15), gly-arg-gly-glu-ser (GRGESP)(SEQ ID NO: 16) (Sigma Chemicals, St. Louis, Mo.). Monoclonal antibodies (mAbs) to the human CD29 molecule (β₁-integrin), LFA-1, α2, α4, and α5 chains of the VLA integrins (Serotec, Oxford, UK). Rabbit anti-D3 receptor antibody (Calbiochem, San Diego, Calif.). Mouse anti-human CD29 antibodies clone 3S3 (Serotec, Oxford, UK), B44 (Chemicon, Temecula, Calif., USA). Anti-rabbit IgG (Jackson ImmunoResearch Laboratories, Inc., West Grove, Pa.); Phycoerythrin (PE)-conjugated anti-TCR (Serotec, Oxford, UK). Taq polymerase (Promega Corp., Madison, Wis., USA).

Human T-cells: Human T-cells were purified from the peripheral blood of healthy donors as follows: the leukocytes were isolated on a Ficoll gradient, washed, and incubated on petri-dishes to remove monocytes (37° C., 10% CO₂-humidified atmosphere). Two hours later, the non-adherent T-cells were collected and incubated on nylon-wool columns (Novamed LTD, Israel). Non-adherent T-cells were eluted, washed, and passed through human CD3⁺ cell purification columns (Cedar-Lane, Canada). The resulting cell population consisted of >92% T-cells, as evaluated by CD3 staining regularly performed in all the experiments.

Analysis of Gene Expression Using the Human Atlas cDNA Expression Array:

Poly A+RNA was extracted from human T-lymphocytes before and after treatment with 10 nM DPAT for 24 hours, using the Atlas Pure Total RNA Labeling System (Clontech Laboratories, Inc. Palo Alto, Calif.) according to manufacturers recommendations. Following DNase treatment, ³²P-labeled cDNA was prepared from poly A+ RNA preparations that were prepared from either untreated or Dopamine treated human T-cells. Hybridizations to the Atlas Human cDNA Expression Arrays membranes (Catalog No. 1.2 KIII (7850-1) and III (7855-1), Clontech Laboratories) were performed by Clonetech Laboratories, as described in the user manual, and the expression pattern of up and down regulated genes was visualized by autoradiography.

T-cell adhesion assay: In the majority of the experiments, adhesion of purified normal human T-cells to fibronectin was assayed with radioactively labeled T-cells as follows: normal human T-cells, purified from a fresh blood sample, were labeled with Na₂[⁵¹CrO₄], washed, and resuspended in adhesion medium (RPMI-1640 supplemented with 2% BSA, 1 mM Ca⁺⁺, 1 mM Mg⁺⁺, 1% sodium pyruvate, 1% glucose, and 1% HEPES buffer). The cells were then pre-treated (30 min., 37° C.) with DPAT, and added to fibronectin coated (1 μg/well; Sigma Chemicals, St. Louis, Mo.) microtiter flat-bottomed 96 well plates (1×10⁵ cells/100 μl/well). The plates were placed in a humidified incubator (37° C., 30 min., 10% CO₂), and than washed thoroughly several times with PBS, to remove non-adherent T-cells. The adherent T-cells were lysed with 1% Tween 20 in 1N NaOH, and the radioactivity in the resulting supernatants was determined in a γ-counter. For each experimental group, results were expressed as the mean CPM±SD of bound T-cells from quadruplicate wells. DPAT-treated T-cell adhesion to BSA-(rather than fibronectin) coated wells, and untreated T-cell adhesion to fibronectin coated wells, were the standard negative controls.

Non-radioactive T-cell adhesion assays: Alternatively, T-cell adhesion assays were performed with non-radioactively labeled cells, differing from the above protocol only in the final detection steps which were performed as follows: After the final washing of the adhesion plates and removal of the non adherent cells, the microtiter wells were supplemented with lysis and substrate solution (60 μl/well of: 0.5% Triton X-100 in water mixed with an equal volume of 7.5 mM p-nitrophenol-N-acetyl-β-D-glucosaminide, (Sigma Chemicals, St. Louis, Mo.) in 0.1M citrate buffer pH=5.0). The plates were then incubated (˜18hr) in a CO₂-free 37° C. incubator, supplemented with stop solution (90 μl/well of 50 mM glycine, (Sigma Chemicals, St. Louis, Mo.) pH=10.4 containing 5 mM EDTA), and the optical density was measured at 405 nm in a standard Elisa reader.

Inhibition of Dopamine-induced T-cell adhesion by specific antagonists: ⁵¹[Cr]-labeled T-cells were pre-treated with Dopamine receptor antagonists (10⁻⁶M) for two minutes prior to exposure to Dopamine or DPAT (10⁻⁸M, unless specified otherwise). The treated cells were suspended in adhesion medium and incubated (30 min., 37° C.) in a humidified 10% CO₂ incubator. The cells were seeded in the fibronectin-coated microtiter plates, and the plates were then returned to the incubator for an additional 30 min. incubation. The amount of T-cell adhesion was determined as above.

Inhibition of Dopamine-induced T-cell adhesion to fibronectin with specific integrin blockers: ⁵¹[Cr]-labeled T-cells were pre-treated (30 minutes) either with the RGD- or the RGE-containing peptides (50 μg/ml), or with mAbs (15-25 μg/ml) specific to the human integrins (CD29, LFA-1, and α2, α4, and α5 chains of the VLA integrins). The T-cells were then treated (30 minutes) with either Dopamine or DPAT (10⁻⁸M) and incubated (30 minutes, 37° C., 10% CO₂ humidified incubator). The treated cells were seeded in fibronectin-coated microtiter plates. The plates were returned to the incubator for an additional 30 minute incubation and T-cell adhesion was determined as previously described.

RT-PCR: Total RNA was extracted from SJL/J anti-MBP 87-99 T-cell line and treated with RNase-free DNase (Promega Corp., Madison, Wis., USA). Total RNA (20 μg) from the T-cells was reverse transcribed into cDNA, and was resuspended in distilled water (20 ml). A sample of cDNA (2 ml) was than amplified by PCR, using D3-specific primers [5′-GGAATTCCAGGTTTCTGTCAGATGCC-3′](SEQ ID NO. 17) and [5′-GGAATTCCGTTGCTGAGTTTTCGAAC C-3′](SEQ ID NO: 18), based on the sequence of the mouse D3-Dopamine receptor. PCR amplification was performed using Taq polyrnerase (2.5 units, Promega Corp., Madison, Wis., USA) in a reaction volume (100 μl) containing 50 mM KCl, 10 mM Tris-HCl, pH 8.8, 1.5 mM MgCl₂, 0.1% Triton X-100, 300 mM of each dNTP and 50 pmole of each primer. PCR was performed in a DNA thermal cycler (Progene, Techne, Inc., Princeton, N.J., USA): after initial denaturation (94° C., 5 min), each cycle consisted of a denaturation step (94° C., 1 min), annealing step (58° C., 1 min) and extension step (72° C., 1 min). Number of cycles were either 25, 30 or 35.

Immunofluorescence staining for Dopamine D3 receptor: Murine anti MBP 87-99 T-cells, or normal human T-cells isolated from fresh PBLs were stained by indirect immunofluorescence, using a rabbit anti-D3 Dopamine receptor antibody (Calbiochem, San Diego, Calif.) (100 μl of 1:100 dilution per 1-3×10⁶ cells/tube, 30 min. on ice), and FITC-conjugated anti-rabbit Ig (The Jackson Labs, Bar Harbor, Mass., USA). The cell fluorescence following incubation with only the secondary FITC-conjugated anti-rabbit Ig, or with non-relevant antibody were standard negative controls, considered as non-specific control staining. Fluorescence profiles were recorded in a Fluorescence-activated cell sorter (FACSSORT, Becton and Dickinson, San Jose, Calif., USA).

Membrane potential measurements by flow cytometry: Purified normal human or mouse antigen-specific T-cells were incubated in serum-free medium (RPMI, ˜1×10⁶/ml, 1 hr, 37° C., 10% CO₂ humidified incubator) to assure steady state conditions. The cells were then washed, resuspended in normal RPMI, distributed into individual tubes (˜1×10⁶/tube), added with 300 nM di-BA-C₄(3) oxanol dye (Molecular Probes, Inc., Eugene, Oreg., USA), and 5-15 minutes later added with DPAT (10⁻⁸ M). Alternatively, the human T-cells were resuspended either in normal RPMI, or in rich K⁺ RPMI solutions, incubated for 30 min (37° C., 10% CO₂), and loaded with 300 nM di-BA-C₄(3) oxanol dye. The cell fluorescence was analyzed on the FACSORT at 488 nm.

Immunofluorescence staining for activated β1 integrins: Freshly purified normal human T-cells were washed, counted, and 5×10⁵ cells added with DPAT (10⁻⁸M) and placed in a humidified incubator (37° C., 30 min. 10% CO₂). The cells were then added with the anti-activated β1 integrin HUTS 21 mAb (5 ml/sample, A kind gift from F. Sanchez-Madrid), returned to the 37° C. incubator for 10 minutes, and then transferred to 45 min. incubation in 4° C. The cells were then washed, resuspended in a PBS, and added with FITC-conjugated goat anti-mouse antibody (1 mg/sample, 45 min, 4° C.). Following a final wash, the cells were resuspended in cold staining buffer (PBS, 1% FCS, 0.01% sodium azide) and analyzed by FACSORT (Becton and Dickinson, San Jose Calif., USA).

Immunoprecipitation and immunoblottinig: Preparation of membrane fractions from fresh purified normal human T-cells, immunoprecipitations and immunoblottings were performed as follows: the β1 integrins were immunoprecipitated by a mouse anti-human CD29 antibody (clone 3S3, Serotec, Oxford, England), and immunoblotted with a different mouse anti-human CD29 antibody (clone B44, Chemicon, Temecula, Calif., USA), according to the manufacturers instructions. The Dopamine D3 receptor was immunoprecipitated and immunoblotted using a rabbit anti-D3 receptor antibody (Calbiochem, San Diego, Calif.).

Initiation and propagation of mouse antigen-specific T-cell lines: Anti MBP 87-99 and anti-PLP 139-151-specific CD4⁺ T-cell lines were established from lymph nodes of SJL/J mice, preimmunized in both hind foot pads with the PLP peptide (100-200 μg/mouse) emulsified in 4 mg/ml of Mycobacterium tuberculosis, in complete Freund's adjuvant (CFA, Difco, Detroit, Mich., USA). Lymph nodes were removed 10 days post immunization, cultured, and antigen-specific T-cells selected in vitro using the immunizing peptide (5-10 μg/ml) in art-recognized, antigen-based screening techniques.

Induction of experimental autoimmune encephalomyelitis (EAE) by encephalitogenic T-cells: Following 72 hours antigenic-stimulation, 3-5×10⁵ of anti-PLP 139-151 T-cells suspended in PBS were injected IV into the tail vein of naive SJL/J female mice. The recipient mice were examined daily for clinical signs of the EAE, and were graded according to the following scale: 0. no abnormality; 1. loss of tail tone; 2. weakness of one hind limb; 3. total paralysis of both hind limbs; 4. total paralysis of both fore limbs; 5. premoribund state; 6. death. The individual grading the EAE was unaware of the identity of the mice groups.

Passively transferred delayed type hypersenisitivity (DTH): Groups of 6-8 BALB/c female mice (The Jackson Laboratory, Bar Harbor, Mass., USA) were sensitized on the abdominal skin with 200 μt of 2% oxazalone dissolved in acetone/olive oil [4:1 vol/vol)] applied topically. Five days later, lymph nodes were removed from the sensitized mice, and a cell suspension prepared. The cells (3-5×10⁶/ml in RPMI, no additional supplements) were than added for 1 hr incubation (30 min. 37° C., 10% CO₂ humidified incubator) with either DPAT, U-Mal, or both (in the latter combination the antagonist added 5 minutes before the agonist). The cells were than washed, resuspended in PBS, and injected IV (40×10⁶ /mouse) into normal BALB/c recipients. Immediately afterwards the recipient mice were sensitized with 10 μl of 2% oxazalone in acetone/olive oil, applied topically to each side of the ear. A constant area of the ear was measured immediately before challenge and 24 hr after the challenge with a Mitutoyo engineer's micrometer. The individual measuring the ear swelling was unaware of the identity of the mice groups. The DTH reaction is expressed as the mean±SD (for each group) of the increment of ear swelling 24 hours after inoculation of the oxazalone-sensitized T-cells, in units of 10⁻² mm.

Statistical Analysis:

Statistical analysis was performed by standard t-test.

### EXAMPLE I

### Dopamine Induces T-cell Adhesion to Fibronectin, by Direct Interaction with its Receptors

Can Dopamine interact directly with specific Dopaminergic receptors on normal human T-cells, activate β1 integrin function, and drive the cells into integrin-mediated adhesion to fibronectin, a major glycoprotein component of the extra cellular matrix (ECM)? To the best of our knowledge, Dopamine by itself was never shown to activate T-cell function.

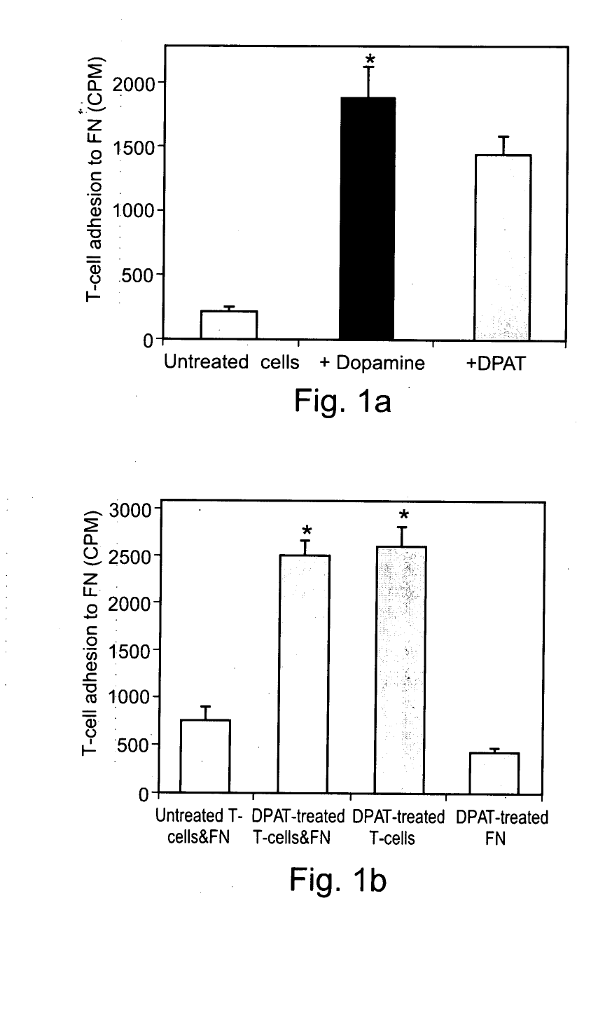

The results, presented in FIG. 1A, show that Dopamine, in the absence of any additional molecules, induce substantial adhesion of normal human T-cells to fibronectin. Since only activated T-cells can adhere to fibronectin, these results suggest that Dopamine is able to activate T-cells in a manner that activates their β1 integrins. FIG. 1A also shows that the T-cell activating effect of Dopamine was mimicked by 7-Hydroxy-DPAT (DPAT), which is considered a selective Dopamine D3 receptor agonist, although it was recently shown to have the ability to bind also the D2 receptor, albeit to a much lesser extent. These results demonstrate, for the first time, that Dopamine and Dopamine analogs can trigger T-cell adhesion to fibronectin by activating the D3 and possibly also the D2 receptor subtypes.

Do Dopamine and DPAT interact directly with the T-cells, or are their pro-adhesive effects mediated by an interaction with fibronectin itself ? The results presented in FIG. 1B show that the pro-adhesive effect of DPAT was mediated by the direct interaction with the Dopamine receptors on T-cells, rather than by an interaction with fibronectin, since a significant adhesion of T-cells to fibronectin was evident only if the T-cells, (not the fibronectin-coated plates), were treated with DPAT. Furthermore, FIG. 1B shows that a transient stimulation of the Dopamine receptors on T-cells was sufficient to activate the β1 integrins and cause the cells to adhere to fibronectin, since the extensive washing of the DPAT-treated T-cells and the removal of free DPAT prior to T-cell seeding on the fibronectin-coated plates, did not affect the final outcome.

### EXAMPLE II

### T-cell Activation by Dopamine and DPAT is Dose Dependent at Physiological Concentrations

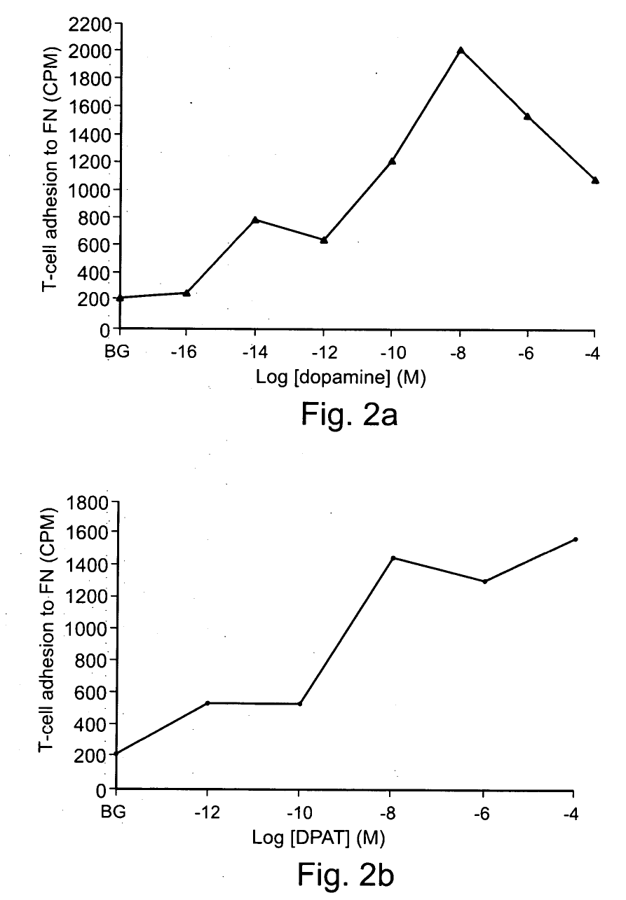

In order for the observed effects of Dopamine on T-cell binding to fibronectin to be of physiological significance, increased response with greater concentrations and a concentration optimum consistent with actual physiological concentrations must be demonstrated. FIGS. 2A and 2B illustrate the dose-dependent nature of the T-cell adhesion induced by both Dopamine and DPAT, with an optimum reached at 10 nM, consistent with concentration optima observed in previous studies on the direct interactions of several neurotransmitters with T-cells (Clark, E. A. and Brugge, J. S., Eur J Pharmacol 1995. 272: RI-3; and Levite, M., Cahalon, L., Hershkoviz, R., Steinman, L. and Lider, O., J. Immunol 1998. 160: 993-1 000). Thus, T-cells are clearly responsive to physiological concentrations of the neurotransmitter Dopamine and it's analogs.

### EXAMPLE III

### Dopamine-Induced T-Cell Adhesion to Fibronectin is Mediated by the α₄β₁ and α₅β₁ Integrins

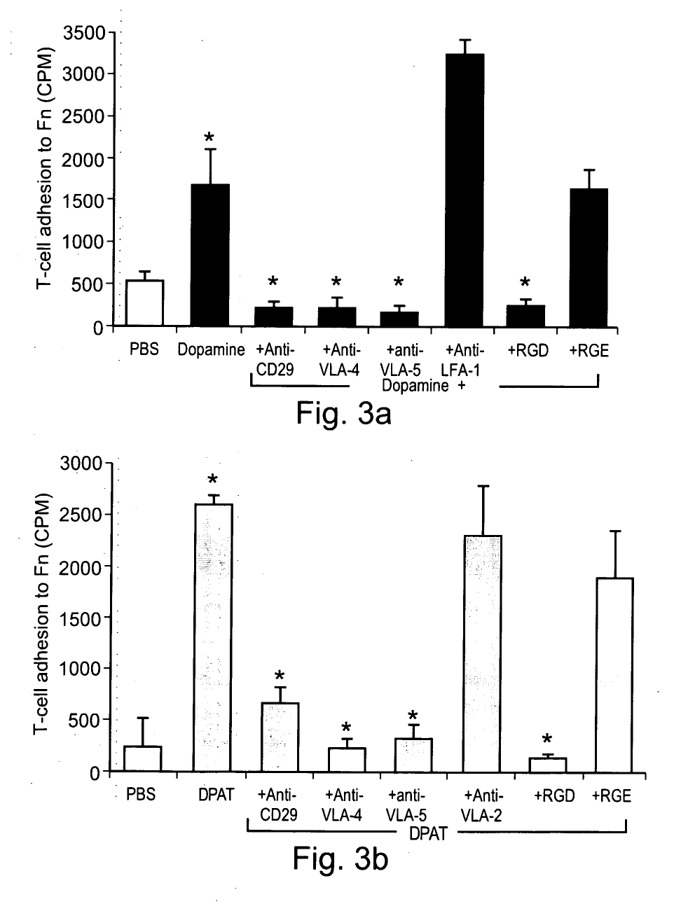

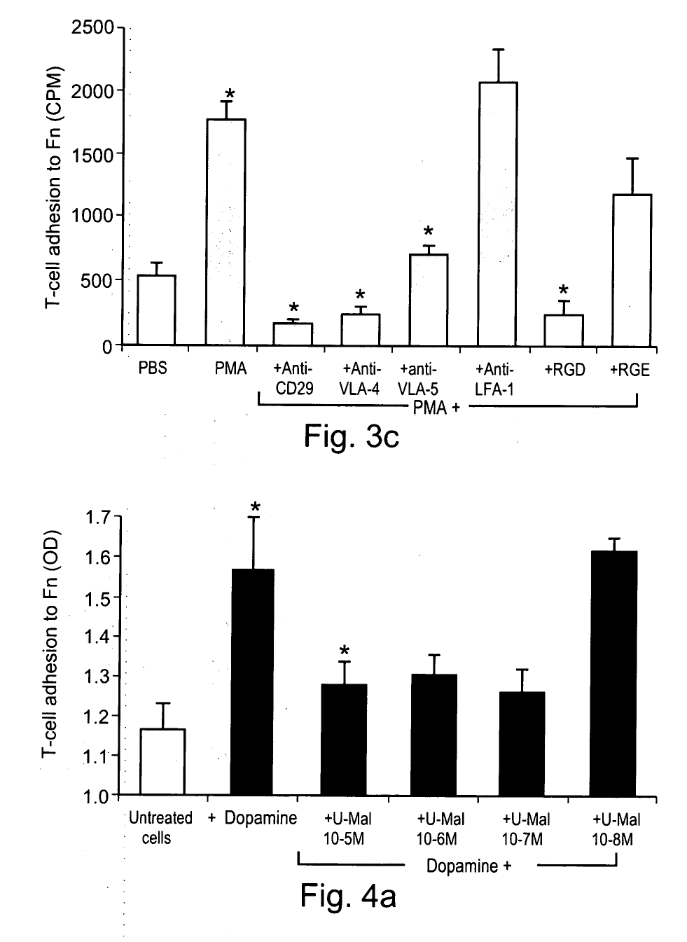

To show that the triggering effect of Dopamine on T-cell adhesion to fibronectin is mediated by specific interaction of fibronectin with the T-cell α₄β₁ and α₅β₁ integrins, as for other physiological stimuli, we made use of monoclonal antibodies (mAbs) specific to the α₄β₁ and α₅β₁ integrin moieties, and of control antibodies directed against other non relevant integrin moieties. We also used an RGD-containing peptide, the region specifically involved in the binding of the α₄β₁ and α₅β₁ integrins to fibronectin, to specifically block (by virtue of competition) the β1 integrin-fibronectin adhesive interactions, and a non-relevant RGE-containing peptide as a control. The results, presented in FIGS. 3A and 3B show that the effects of Dopamine and of its D3 receptor agonist, DPAT, were effectively blocked by anti-VLA-4 (CC4 integrin chain), VLA-5 (α₅ chain) and CD29 (β₁ chain) mAbs, and by the RGD peptide. No significant effect was exerted by the non-relevant anti-VLA-2 (α₂β₁) and -LFA-1 (αLβ2) mAbs and by the RGE-containing peptide. T-cell adhesion to fibronectin induced by the phorbol ester PMA, shown in FIG. 3C and serving as a routine positive control, shows a similar profile of inhibition by the relevant monoclonal antibodies and RGD peptide, and insensitivity to exposure to the control anti LFA-1 and RGE peptide.

Thus, Dopamine and DPAT clearly induce adhesion of T-cells to immobilized fibronectin via the characteristic mechanism of specific recognition and binding of fibronectin to the α4β1 and α5β1 T-cell integrins.

### EXAMPLE IV

### Dopamine Stimulates T-Cell Adhesion to Fibronectin through its D3 and D2 Receptors Subtypes

To confirm that Dopamine exerts an activating effect on T-cells thorough binding to its D3 and possibly also D2 receptors, T-cells were exposed to several well-defined and selective Dopamine receptor agonists and antagonists (Table 1), acting primarily on the D2-like receptors. These Dopamine receptor analogs were used at a range of concentrations reported to be effective for the specific activation or suppression of distinct Dopamine receptor subtypes.

TABLE 1
Specificity of Dopamine
receptor agonists and antagonists.

| | Functional | Selectivity to |
|---|---|---|
| Dopamine | receptor | receptor |
| receptor analog | agonist/antagonist | subtype |
| 7-OH-DPAT | Agonist | D3>>>>D2 |
| Bromocryptine | Agonist | D2 |
| Pergolide | Agonist | D1/D2 |
| U-Mal | Antagonist | D3 |
| Butaclamol | Antagonist | D2/D1 |
| Haloperidol | Antagonist | D2/D1 |
| Pizomide | Antagonist | D2 |

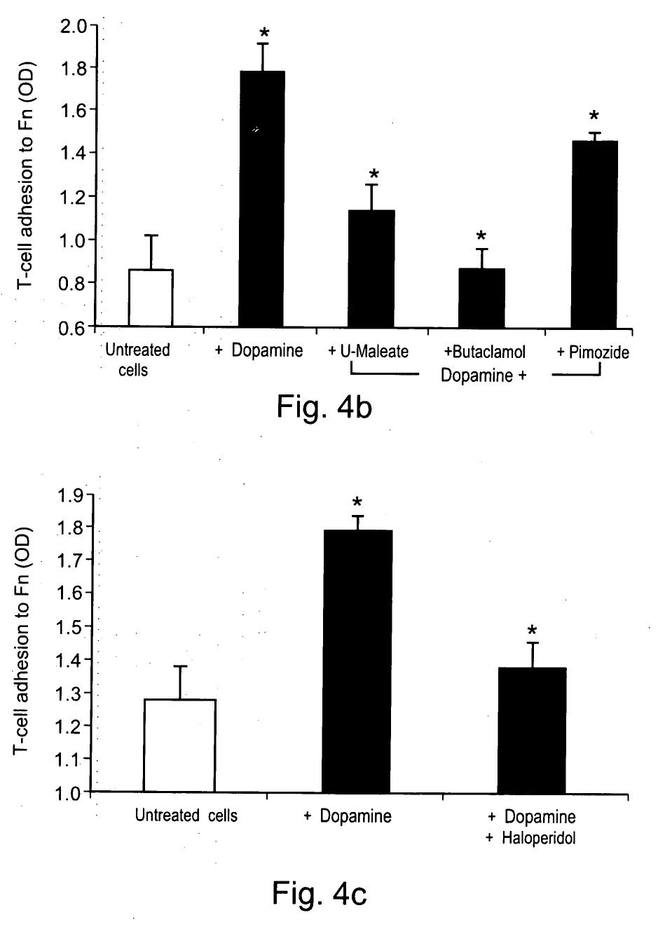

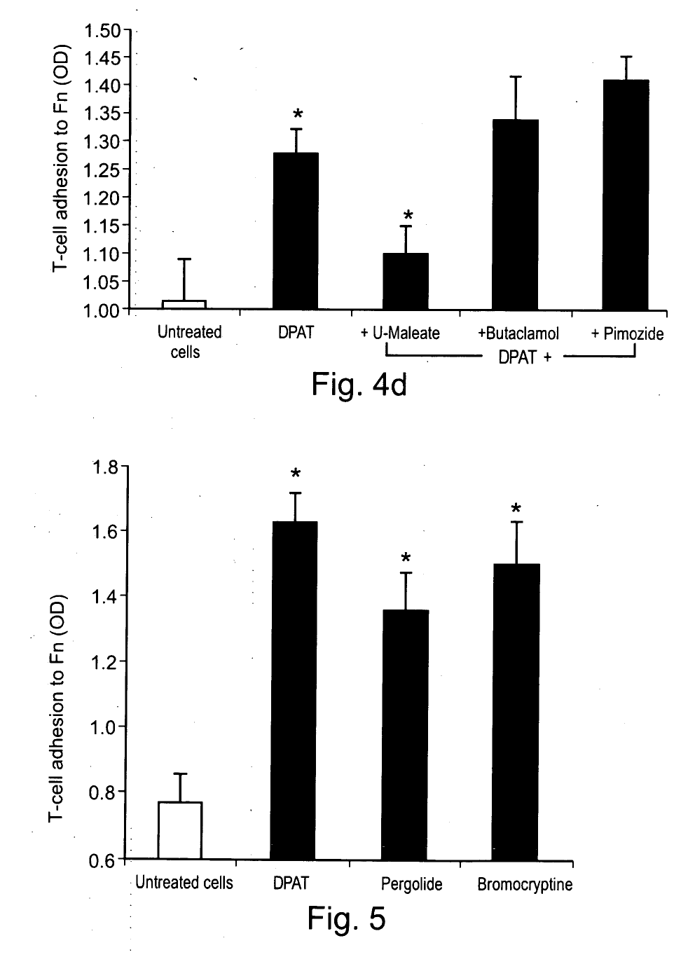

Using the specific D3 receptor antagonist U-991941 Maleate (U-Mal), it was shown (FIG. 4A) that Dopamine induction of T-cell adhesion to fibronectin is completely blocked by pretreatment with the specific D3 receptor antagonist, in a dose dependent manner. U-Mal had no toxic effects on the human T-cells, and no independent effect on T-cell adhesion to fibronectin (data not shown).

The possibility that Dopamine induces T-cell adhesion via additional receptor subtypes, such as the D2, is strengthen by the observations depicted in FIG. 4B, where Butaclamol, an antagonist of the Dopamine D2/D1 receptors, fully blocked the effect of Dopamine. However, Pimozide, a D2 receptor antagonist was less effective in blocking Dopamine induction than Butaclamol (FIG. 4B), while Haloperidol, a D2/D1 receptor antagonist, behaved as Butaclamol, fully blocking the Dopamine-induced adhesion (FIG. 4C). When T-cell adhesion was induced by the selective D3 agonist, DPAT, rather than by Dopamine, only the D3 receptor antagonist U-Mal succeeded in blocking this effect, while Butaclamol and Pimozide were without effect (FIG. 4D).

To complement the results demonstrated with specific Dopaminergic receptor antagonists, the role of D2 receptors in the pro-adhesive effect induced by Dopamine was assessed using the Dopamine receptor agonists Bromocriptine (D2) and Pergolide (D1/D2). The results depicted in FIG. 5 clearly demonstrate these agonists' Dopamine-independent capability to induce T-cell adhesion to fibronectin, although less effectively than DPAT.

### EXAMPLE V

### Dopamine D3receptor Transcripts and Receptors are Found in Human and Mouse T-Cells

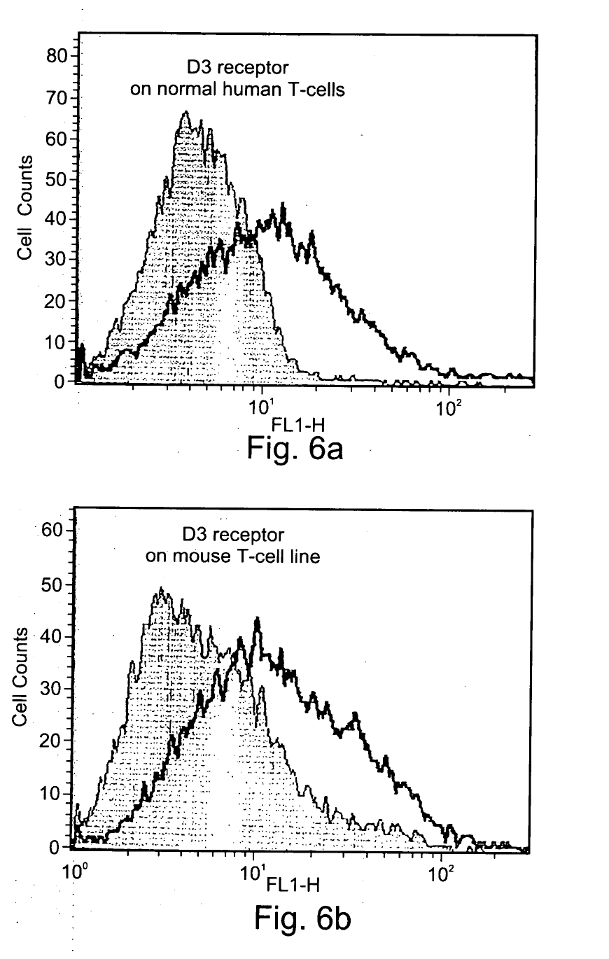

While the effects of specific receptor-type Dopamine agonists and antagonists on T-cell function disclosed herein demonstrate the role of such cell-surface receptors in Dopamine's modulation of T-cell function, definitive evidence of their presence has been lacking. While reducing the present invention to practice, it was uncovered, for the first time, that normal human (FIG. 6A) and mouse antigen-specific (FIG. 6B) T-cells express an immune-reactive Dopamine D3 receptor, recognized by rabbit anti-D3 Dopamine receptor antibody.

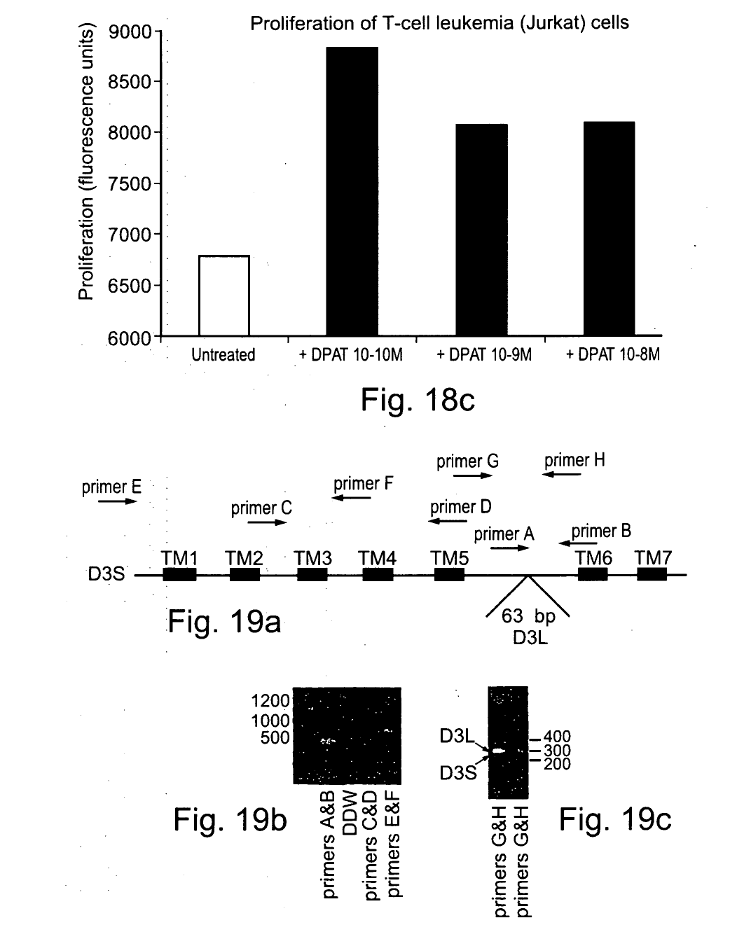

That the Dopamine D3 receptor expressed in T-cells is identical to D3 receptor subtypes identified from other cell types is demonstrated by the RT-PCR amplification of D3 subtype transcripts from mouse antigen-specific T-cells, using Dopamine D3 receptor-specific primers (FIGS. 19A-19C). These results are consistent with the predominance of D3-mediated effects of Dopamine on T-cells disclosed hereinabove, indicating that the D3 receptor is crucial to Dopamine's effects on both resting and activated T-cell function.

### EXAMPLE VI

### DopamineD3 Receptor-Mediated Depolarization of Human Aged Mouse T-Cells

As noted hereinabove, Dopamine, and Dopamine agonists, are known to cause changes in trans-membrane potentials, and hence, the polarization of excitable membranes, such as neuronal membranes. In order to further elucidate possible mechanisms of Dopamine's action on T-cell function, changes in membrane potential were analyzed by flow cytometry, using the voltage-sensitive oxanol dye, DiBAC₄(3), the fluorescence intensity of which correlates with the membrane potential. Depolarization of the human T-cells is by increasing concentrations of extracellular K+served as positive control.

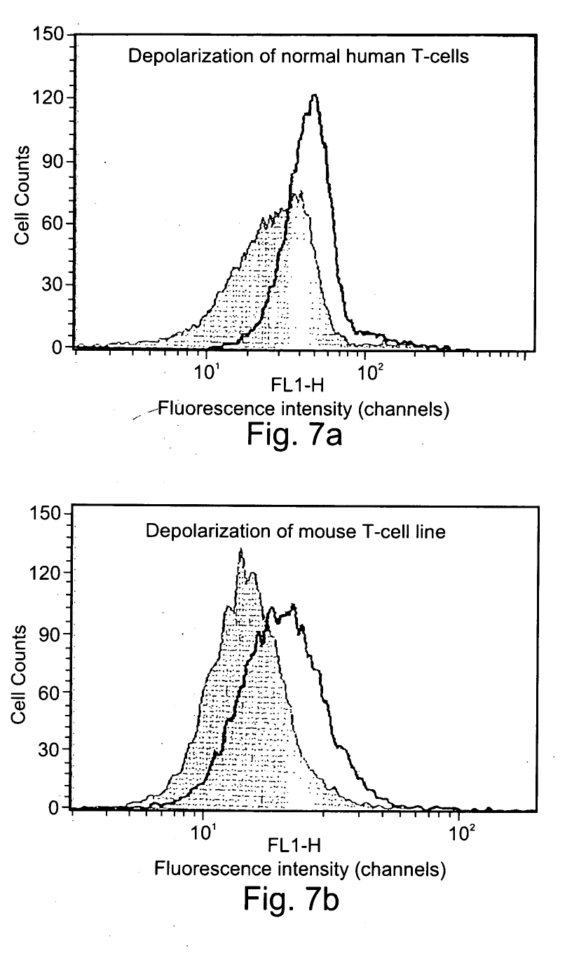

Exposure of dye-loaded resting normal Human (FIG. 7A) and anti-MBP-specific T-cell line (FIG. 7B) to 10⁻⁸ M DPAT results in a shift to the right of the fluorescence profile, indicating that DPAT causes a clear depolarization of both normal human T-cells and mouse antigen specific T-cells. The level of DPAT-induced T-cell depolarization, was comparable to that caused by ˜20 mM extracellular K⁺ (FIG. 7C), corresponding to a shift of ˜6mV from a resting potential of ˜48 mV.

The depolarization of T-lymphocytes by DPAT, suggests that the activation of Dopamine D3 receptors is likely to affect voltage sensitive cellular processes, among them the opening of the voltage-gated-potassium channels.

### EXAMPLE VII

### Dopamine Suppression of T-Cell-Mediated Inflammatory Disorders Via D3 and D2 Receptors

Suppression of T-cell induced Experimental Autoimmune Encephalomyelitis (EAE) With DPAT. Some biological effectors have a dual regulatory effect, depending on the activation state of the target cells, such as suppressing in vivo immunoreactivity of activated or sensitized T-cells. In order to determine whether Dopamine stimulation of T-cells has such a context-dependent character, cultured mouse T-cells directed against peptide determinants of the myelin proteolipid protein (SJL/J anti PLP 139-151), which, following antigenic stimulation, can induce severe experimental autoimmune encephalomyelitis (EAE), a model for Multiple Sclerosis, in naive recipient mice which were exposed to DPAT for one hour, before inoculation into naive recipient mice.

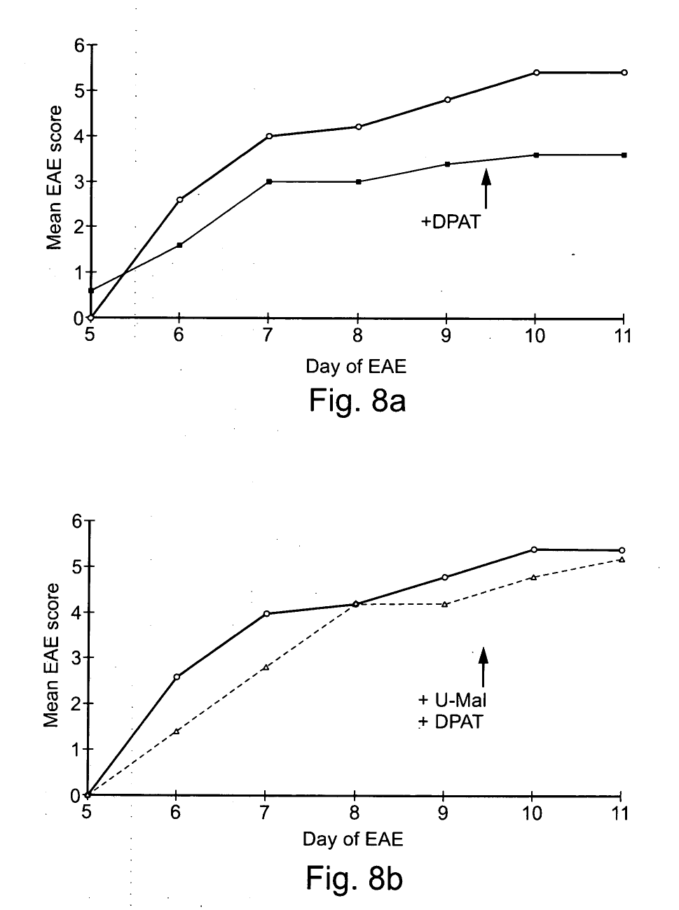

While reducing the present invention to practice, it was uncovered that a direct, short-term and transient ex-vivo interaction between the antigen-activated anti PLP T-cells and DPAT significantly reduced the severity of pathogenic response in vivo, documented 5-12 days later (FIG. 8A). The DPAT suppressive effect was prevented by a pre-treatment with the Dopamine D3 receptor antagonist U-Mal (FIG. 8B), indicating a D3 receptor-mediated mechanism. Thus, DPAT suppressed the in vivo activity of activated T-cells.

DPAT suppressive effect on EAE-inducing T-cells was obtained in all four independent experiments performed, and was manifested both in the mean severity of EAE (representative experiment shown in FIG. 8A) as well as in the percentage of mice developing the disease. Thus, while out of all the mice injected with the untreated anti-PLP 139-151 T-cells throughout the four independent experiments 95% (the mean of 100%, 80%, 100% and 100%) developed severe EAE, only 70% (the mean of 83%, 60%, 80% and 60%) of the mice injected with DPAT-treated T-cells were affected by the disease (T-test p<0.01 1). It is noteworthy that the reduction in the in vivo pathogenicity of the cells following DPAT exposure was observed despite the extreme aggressiveness of this particular cell line (leading in many cases to death of the EAE-afflicted mice), and despite the brief and single pulse with DPAT. Such an ex vivo pulse'should in fact be ‘memorized’ by the cells for many days to come, if stable supression of their in vivo pathogenicity is to be achieved

Suppression of T-cell induced Delayed Type Hypersensitivity (DTP) with DPAT. To test whether a direct interaction of activated T-cells with DPAT can suppress an additional in vivo T-cell mediated inflammatory reactivity, T-cells pre-sensitized with oxazalone, and therefore capable of transferring delayed-type-hypersensitivity (DTH), were subjected to a direct, short-term, transient treatment with DPAT, before their inoculation into naive recipient mice.

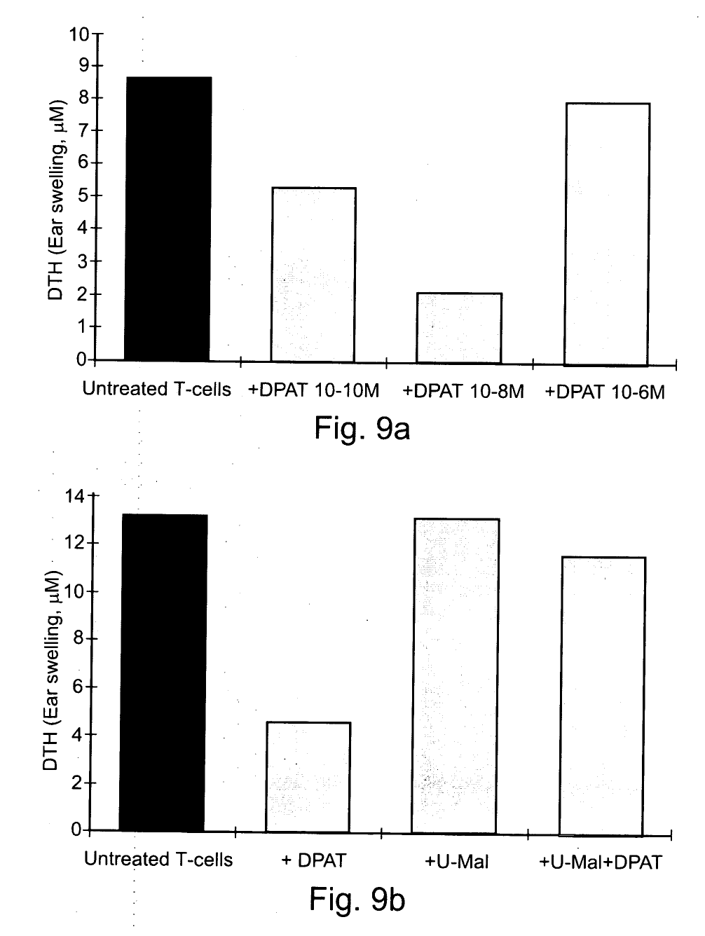

While reducing the present invention to practice, it was shown that is DPAT, at 10⁻⁸M, significantly reduced the DTH caused by the oxazalone-sensitized T-cells (FIG. 9A). Higher and lower concentrations of DPAT were either less or not at all effective. Furthermore, the suppression achieved by the transient interaction with DPAT was prevented by pre-treatment with U-Mal (which had no effect by itself), indicating that it is D3-receptor mediated.

Taken together, the results presented herein show that direct, brief and transient ex vivo activation of the Dopamine D3 receptor, can markedly alter the future in vivo behavior of inflammatory and autoimmune T-cells, the protection enduring for days. Significantly, Dopamine's suppressory effect on previously activated T-cells is clearly distinct from the T-cell activation demonstrated with resting T-cells.

### EXAMPLE VIII

### Dopamine Induces Cytokine Secretion in Resting Human T-Cells

T-cell activation is characterized by numerous responses, such as proliferation, cell binding, chemotaxis and cytokine secretion. It is via the release of specific remote-acting factors such as the cytokines, that the cells of the immune system communicate with each other to coordinate appropriate immune and inflammatory responses. “Naïve” T-cells, Th0, Th1 and Th2 cells are further characterized by the types of cytokines which they synthesize and secrete: “Naïve” cells typically secrete IL-2, Th0 cells secrete a variety of cytokines, Th1 typically secrete IL-2 and TNF-α, and Th2 secrete IL-4, IL-5, and IL-10. Thus, many normal and pathological conditions are associated with specific cytokine profiles.

Thus, the ability of Dopamine to induce the secretion of specific cytokines in normal and cloned human T-cells was investigated.

FIGS. 10A and 10B show the response of freshly separated human peripheral T-cells when incubated 72 hours in the absence or presence of 10⁻⁸ M Dopamine without antigen stimulation. Whereas none of the typically immunosuppressive Th2 specific cytokine IL-10 was detected by ELISA in the untreated cells (FIG. 10A, Untreated), incubation with 10⁻⁸M Dopamine induced a strong release of IL-10 (FIG. 10A, Dopamine). Furthermore, exposure to Dopamine had only a small (9% increase) effect (FIG. 10B) on the number of IL-10 producing T-helper cells, indicating that induction of IL-10 secretion in these cells, rather than an alteration of the T-cell character, is responsible for the increase of IL-10 observed.

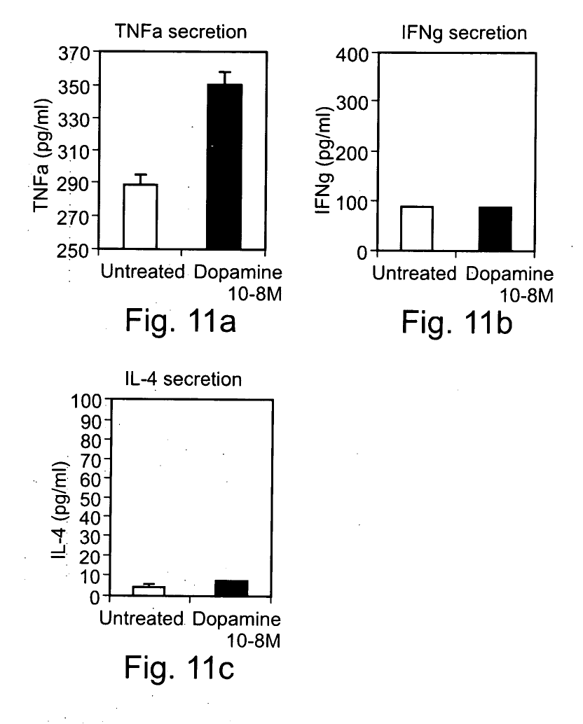

FIGS. 11A and 11B show the response of freshly separated human peripheral T-cells when incubated 24 hours in the absence or presence of 10⁻⁸ M Dopamine without antigen stimulation. Whereas little of the typically inflammatory Th1 specific cytokine TNF-α was detected by ELISA in the untreated cells (FIG. 11A, Untreated), incubation with 10⁻⁸M Dopamine induced a strong release of TNF-α (FIG. 11A, Dopamine). Furthermore, these results demonstrate that this effect of Dopamine on T-cell cytokine release is specific, inducing TNF-α (FIG. 11A), but not IFN-γ (FIG. 11B) or IL-4 (FIG. 11C) secretion.

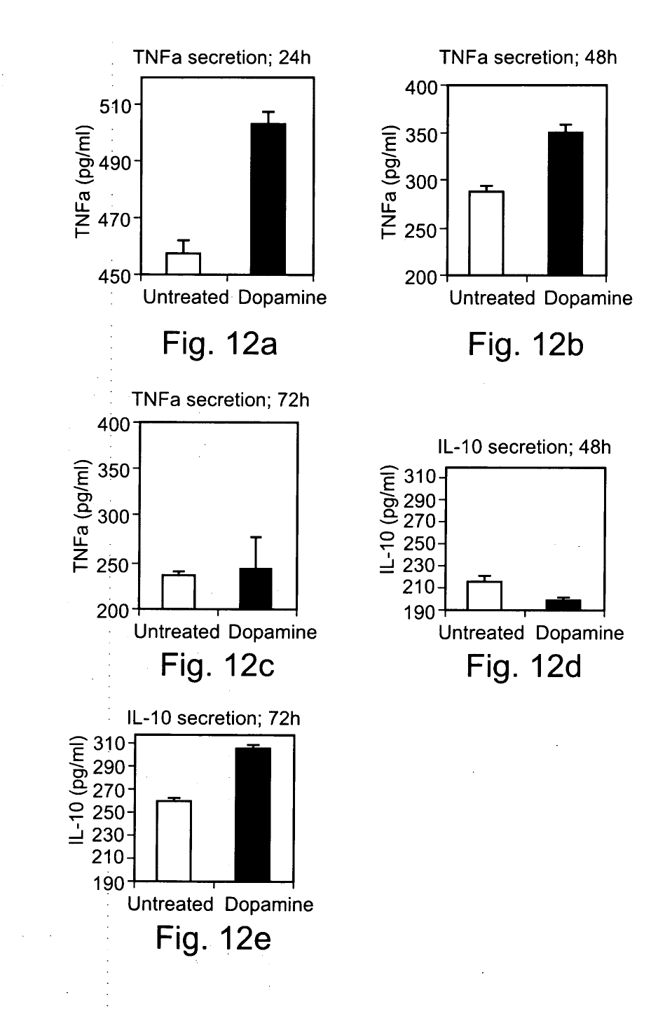

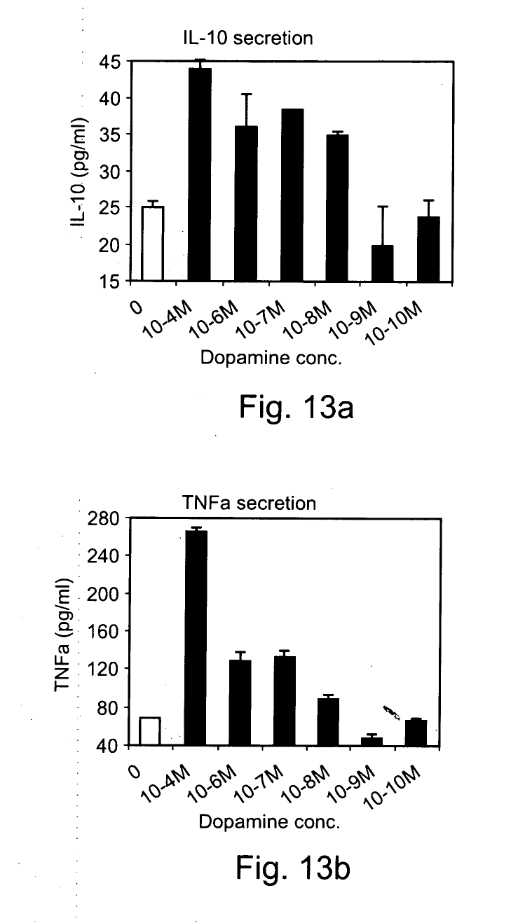

The induction by Dopamine of IL-10 and TNF-α secretion in resting human T-cells was time- (Figure FIGS. 12A-12E) and dose-dependent (FIGS. 13A and 13B). Using low, physiological Dopamine concentrations (10⁻⁸M), induction of TNF-α secretion was maximal at 24 and 48 hours (FIGS. 12B and 12C), while IL-10 secretion was maximal at 72 hours. Although 10⁻⁸M is clearly effective for both IL-10 and TNF-α induction, increasing the Dopamine concentration to 10⁻⁴M caused even greater secretion of both cytokines (FIGS. 13A and 13B, 10⁻⁴M).

Thus, Dopamine alone, at physiological concentrations, induces specific cytokine secretion in resting, unstimulated human T-cells, in a dose- and time-dependent manner.

Dopamine induces “forbidden ” cytokine secretion. Induction of atypical, or “forbidden” cytokine secretion has been observed in vitro, resulting in the “reversion” of T-cell destiny, in the presence of antigens and antigen-presenting cells (Mocci S and Coffman R L J Immunol 1997; 158:1559-64), but also by neuropeptides (Levite M et al PNAS USA 1998;95:12544-54), demonstrating that non-T-cell receptor stimulation participates in the fine tuning of immune and inflammatory responses. However, Dopamine modulation of cytokine profile has never been demonstrated. Thus, the effect of Dopamine on the cytokine profile of resting (no antigen stimulation) human T-cell clones was measured by ELISA using cytokine-specific antibodies.

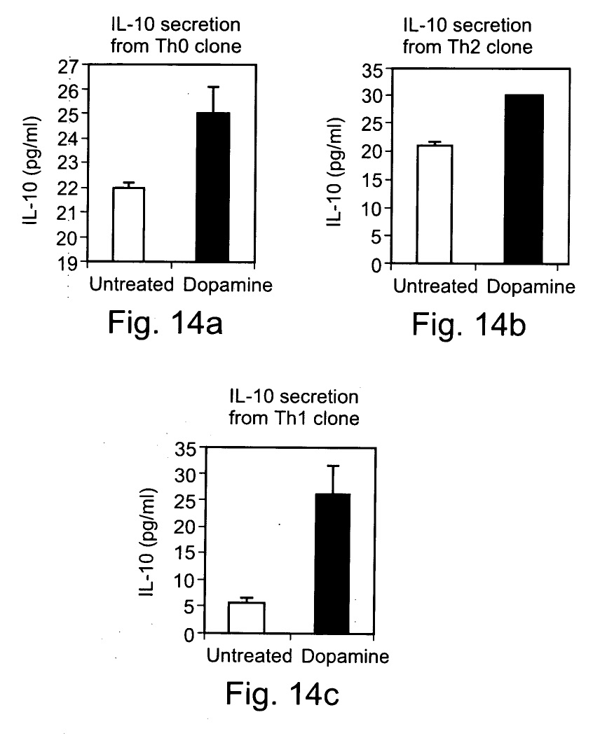

Incubation of cloned, resting human Th0 (FIG. 14A) and Th2 (FIG. 14B) cells with 10⁻⁸ M Dopamine caused a strong induction of “typical” cytokine IL-10 secretion (Dopamine), as compared with untreated cultures (Untreated). Surprisingly, cloned, resting Th1 cells also responded strongly to Dopamine stimulation, secreting significant amounts of IL-10 (FIG. 14C), although IL-10 is typically secreted by Th2 cells, and considered “forbidden” for Th1 cells (Levite M et al PNAS USA 1998; 95:12544-54). Thus, Dopamine alone, in the absence of additional stimulators, directly activates T-cell cytokine secretion in resting human T-cells, and is similarly capable of directly modulating the cytokine profile of committed T-cell lines.

Taken together, these results clearly demonstrate that Dopamine can directly effect IL-10 and TNF-α dependent responses.

### EXAMPLE IX

### Specific Dopamine Receptors Mediate Induction of Cytokine Secretion in Resting Human T-Cells

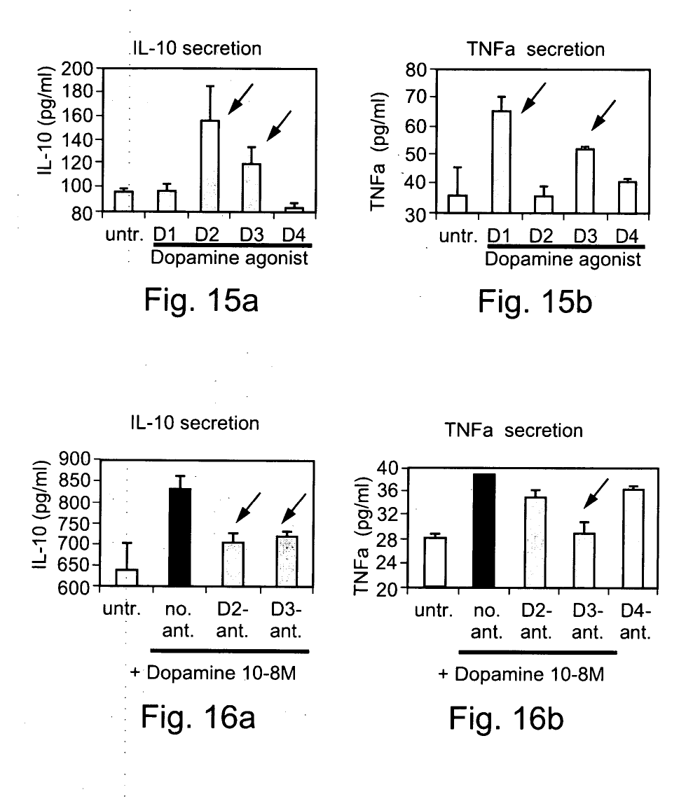

In order to determine the specific Dopamine receptor subtypes (D1-D5) responsible for Dopamine-induced cytokine secretion, and whether Dopamine induces IL-10 and TNF-α production via the same receptor subtype, normal resting human T-cells were exposed to highly specific Dopamine receptor agonists and antagonists (see Table 1). Freshly isolated human peripheral T-cells incubated with 10⁻⁷M Dopamine receptor agonists secreted IL-10 in response to the D2 and, to a lesser extent, D3, but not D1 and D4 receptor agonists (FIG. 15A, black arrows), and TNF-α in response to D1 and D3 receptor agonists, but not D3 or D4 receptor agonists (FIG. 15B, black arrows). Similarly, IL-10 induction was inhibited by D2 and D3 receptor antagonists (FIG. 16A, black arrows), and while TNF-α induction was inhibited by D3 receptor antagonists only (FIG. 16B, black arrows). These results demonstrate, for the first time, that induction of T-cell cytokine secretion, similar to the abovementioned depolarization and fibronectin adherence, is mediated by distinct and specific T-cell Dopamine receptors, and further demonstrate the importance, to T-cell activation, of the individual Dopamine receptor subtypes.

### EXAMPLE X

### Excess Dopamine Induces T-Cell Death in Resting Human T-Cells and Jurkat Leukemia Cells

Neurotoxicity has been reported with exposure of neurons to high concentrations of Glutamate and other neurotransmitters, however, effects of higher than normal, physiological concentrations of Dopamine on T-cell survival are not known. While reducing the present invention to practice, it was uncovered that a high, extremely non-physiological concentration (10⁻³M) of Dopamine caused a drastic reduction in cytokine secretion (FIGS. 17A and 17B). Further investigation revealed, for the first time, that excessively high concentrations (10⁻³M) of Dopamine induces cellular death in normal T-cell lines (FIG. 17C) and Jurkat T-cell leukemia cells (FIG. 17D). Measuring specific cell survival rate at a range of Dopamine concentrations, it became clear that Dopamine concentrations in the range of 2.5×10⁻⁴M to 10⁻³M dramatically impaired both normal human and Jurkat leukemia T-cell survival (FIGS. 17D and 17E).

This important novel demonstration of T-cell death with excess Dopamine is particularly relevant to the clinical use of Dopamine for treatment of certain leukemic conditions.

### EXAMPLE XI

### Dopamine D3receptor Agonist DPAT Induces Proliferation in Resting Normal Human and Jurkat Leukemia T-Cells

Several studies have shown that Dopamine or derivatives thereof can suppress proliferation in activated human peripheral T-cells. Since it has been surprisingly demonstrated, as described hereinabove, that Dopamine exposure of activated T-cells results in immunosuppression (see EXAMPLE VII, FIGS. 8A, 8B, 9A and 9B), while Dopamine clearly stimulates T-cell function in resting T-cells (see EXAMPLES I-VI, VIII-IX), the effect of the Dopamine D3 agonist DPAT on resting T-cell proliferation was investigated.

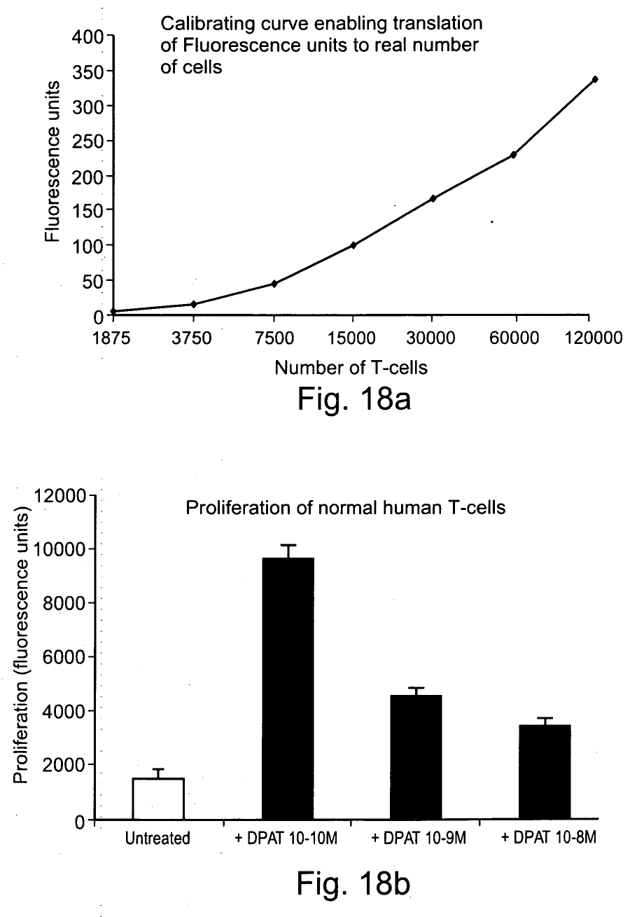

Purified normal human T-cells (FIG. 18B), and cultured Jurkat T-cell leukemia cells (FIG. 18C) exposed to concentrations of DPAT comparable to normal Dopamine plasma concentration (10 pg/ml) and less, exhibited proliferation up to 5 times that of untreated cells (FIG. 1 8B). Note that even extremely low DPAT concentration (10⁻¹⁰ M) were highly effective in inducing resting T-cell proliferation.

Thus, while reducing the present invention to practice, it has been unexpectedly observed that the response of T cells to Dopamine or Dopamine agonists can be context-dependent, resulting in upregulation of T cell activity (cytokine secretion, fibronectin adhesion, proliferation, etc) in resting T cells, and downregulation of certain function (EAE and DTH) in activated T-cells.

### EXAMPLE XII

### T-Cells Respond to Direct Stimulation with Dopamine by Initiation, Modulation or Suppression of De Novo Gene Expression

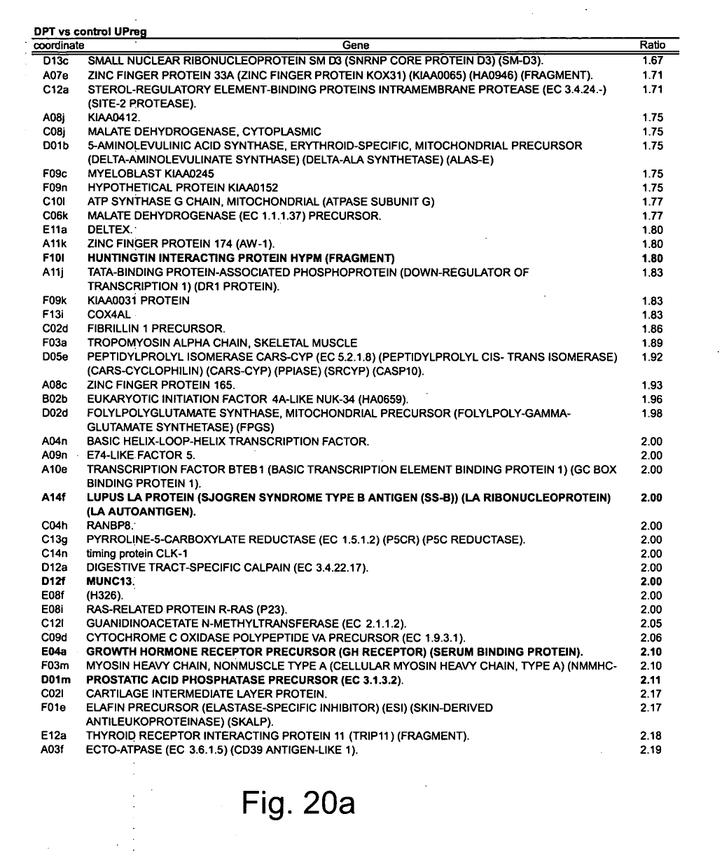

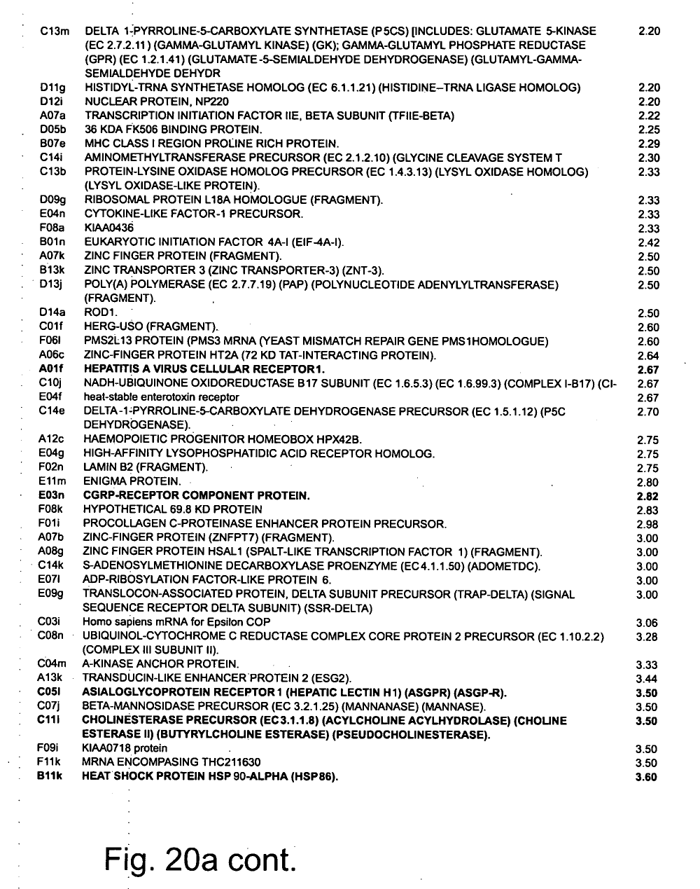

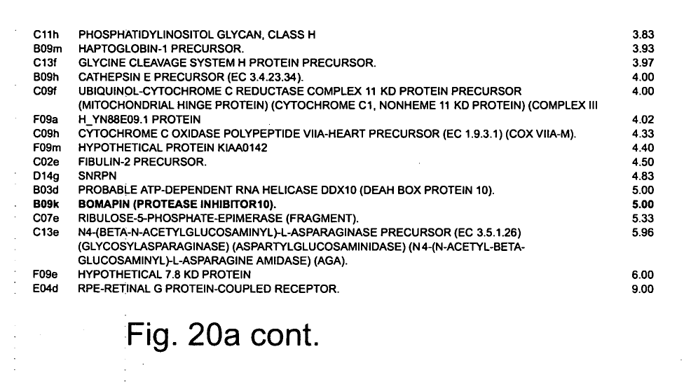

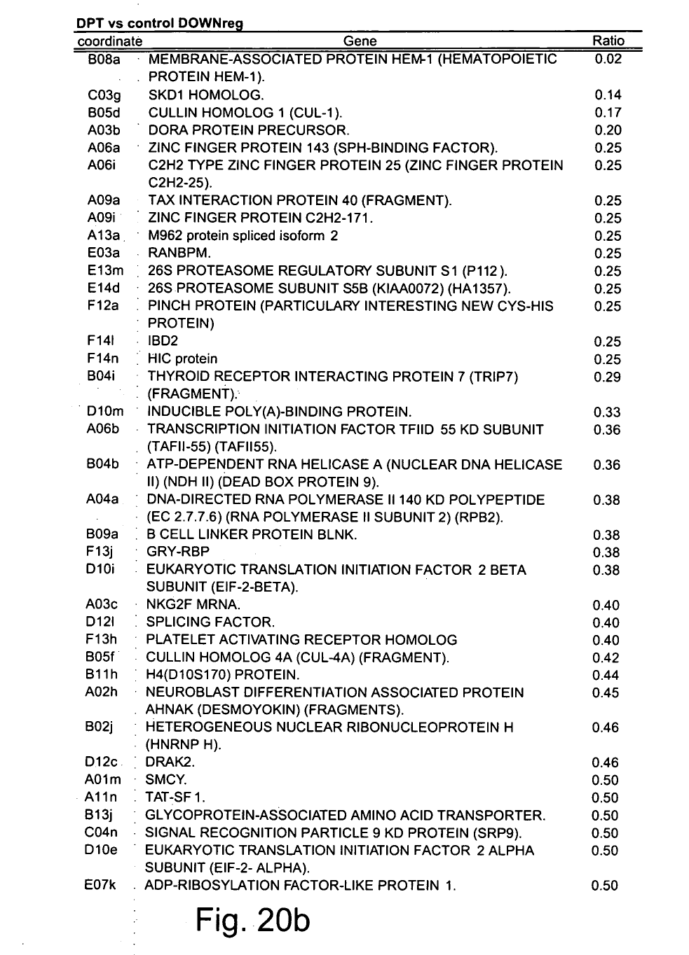

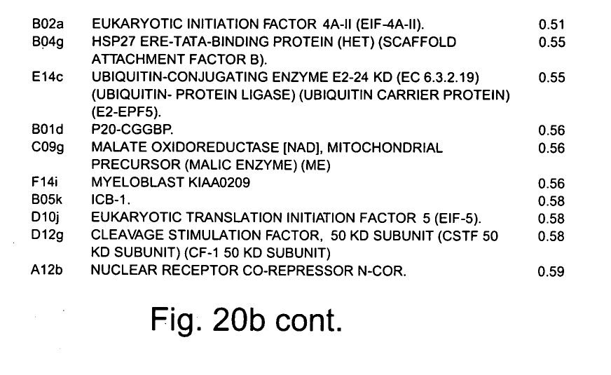

To explore the possible direct effects of Dopamine on gene expression by T-cells, resting human peripheral T-cells were exposed to Dopamine agonist DPAT (10 nM) for 24 hours. Poly A+ RNA was prepared from both DPAT- treated and untreated cells and reverse transcribed to ³²P-labeled cDNA. Using an Atlas human cDNA expression array (i.e. a positively charged nylon membrane spotted with 1200 different human cDNAs) for identification of effected genes, the reverse transcribed products were characterized by hybridization to the atlas membranes. The differential pattern of expression between untreated cells and DPAT-treated cells was visualized by autoradiography, and quantified by densitometry. (FIGS. 20A and 20B). The results revealed that DPAT induced the over expression of mRNA encoding for several genes (FIG. 20A), and down-regulated the expression of others (FIG. 20B). Surprisingly, in addition to modulating the expression of a number of typical T-cell genes (for example, Heat Shock protein 90 and Growth Hormone Receptor protein), exposure to DPAT triggered the expression of a number of genes previously undetected in T-cell. Thus, for example, as noted in FIG. 20A, the neurotransmitter induced expression of Lupus LA protein and Cathepsin E precursor, previously detected in non-lymphoid tissue only. One example of the DPAT's modulation of pathology-related T-cell gene expression is the induction of expression of the serine protease inhibitor Bomapin (protease inhibitor 10, PI 10). This member of the ovalbumin family of serine protease inhibitors is expressed at elevated levels in patients with acute myeloid leukemia and chronic myelomonocytic leukemia, inhibits TNF alpha-induced cell death, and has been linked to the regulation of protease activities in early hematopoiesis (Riewald, M et al Blood 1998;91:1256-62 and Schleef R R and Chuang T L J Biol Chem 2000;275:26385-9). RT-PCR analysis of the mRNA of peripheral T-cells incubated with and without 10 nM DPAT clearly demonstrates the increased abundance of Bomapin transcripts following DPAT treatment (not shown).

Taken together, these results constitute the first demonstration of the direct action of DPAT on T-cell activation, resulting in a DPAT -specific pattern of gene transcription.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### LIST OF REFERENCES

### (Additional References are Cited in the Text)

1 Homykiewics,O. Pharmacol. Res. 1966. 18: 925.
2 Sibley, D. R., Monsma, F. J., Jr. Shen, Y. Int Rev Neurobiol 1993. 35: 391-415.
3 Bunzow, J. R. Van, Tol Hh., Grandy, D. K., Albert, P., Salon, J., Christie, M., Machida, C. A., Neve, K. A. and Civelli, O., Nature 1988. 336: 783-7.
4 Van, Tol Hh, Bunzow, J. R., Guan, H. C., Sunahara, R. K., Seeman, P., Niznik, H. B. and Civelli, O. Nature 1991. 350: 610-4.
5 Sunahara, R. K., Guan, H. C., O'Dowd, B. F., Seeman, P., Laurier, L. G. Ng, G., George, S. R., Torchia, J., Van Tol, H. H. and Niznik, H. B. Nature 1991. 350: 614-9.
6 Seeman, P. and Van Tol, H. H., Curr Opin Neurol Neurosurg 1993. 6: 602-8.
7 Seeman, P. Dopamine Receptors in Human Brain Disease. In Creese, I. and Fraser, C. M. (ed.), Dopamine Receptors. Alan R. Liss 1987, p. 233-245.
8 Seeman, P., Bzowej, N. H., Guan, H. C., Bergeron, C., Reynolds, G. P., ird, E. D., Riederer, P., Jellinger, K. and Tourtellotte, W. W. Neuropsychopharmacology 1987. 1: 5-15.
9 Fink, T. and Weihe, E., Neurosci Lett 1988. 90: 39-44.
10 Weihe, E., Nohr, D., Michel, S., Muller, S., Zentel, H. J., Fink, T. and Krekel, J., Int-J-Neurosci 1991. 59: 1-23.
11 Bergquist, J., Tarkowski, A., Ekman, R. and Ewing, A., Proc Natl Acad Sci USA 1994. 91: 12912-6.
12 Takahashi, N., Nagai, Y., Ueno, S., Saeki, Y. and Yanagihara, T., FEBS Lett 1992. 314: 23-5.
13 Santambrogio, L., Lipartiti, M., Bruni, A. and Dal Toso, R., J Neuroimmunol 1993. 45: 113-9.
14 Barili, P., Bronzetti, E., Felici, L., Ferrante, F., Ricci, A., Zaccheo, D. and Amenta, F., J Neuroimmunol 1996. 71: 45-50.
15 Ricci, A. and Amenta, F. J Neuroimmunol 1994. 53: 1-7.
16 Ricci, A., Veglio, F. and Amenta, F., J Neuroinmmunol 1995. 58: 139-44.
17 Ricci, A., Bronzetti, E., Felici, L., Tayebati, S. K. and Amenta, F., Neurosci Lett 1997. 229: 130-4.
18 Ricci, A., Bronzetti, E., Felici, L., Greco, S. and Amenta, F., J Neuroimmunol 1998. 92: 191-5.
19 Caronti, B., Calderaro, C., Passarelli, F., Palladini, G. and Pontieri, F. E., Life Sci 1998. 62: 1919-25.
20 Bondy, B., de Jonge, S., Pander, S., Primbs, J. and Ackenheil, M., J Neuroimmunol 1996. 71: 139-44.
21 Qiu, Y., Peng, Y. and Wang, J., Adv Neuroimmunol 1996. 6: 223-31.
22 Riskind, P. N., Massacesi, L., Doolittle, T. H. and Hauser, S. L., Ann Neurol 1991. 29: 542-7.
23 Cross, R. J. and Roszman, T. L., J Neuroimmunol 1988. 19: 33-45.
24 Liu, Y. and Wolfe, S. A., Jr., Immunopharmacology 1996. 34: 147-59.
25 Morikawa, K., Oseko, F. and Morikawa, S., Clin Exp Immunol 1994. 95: 514-8.
26 Ilani, T., Ben-Shachar, D., Strous, R. D., Mazor, M., Sheinkman, A., Kotler, M. and Fuchs, S., Proc Natl Acad Sci USA. 98: 625-628.
27 Kwak, Y. T., Koo, M. S., Choi, C. H. and Sunwoo, I., BMC Med Genet. 2: 3.
28 Schmauss, C., Haroutunian, V., Davis, K. and Davidson, M., Proc Natl Acad Sci USA. 1993. 90: 8942-6.
29 Nagai, Y., Ueno, S., Saeki, Y., Soga, F., Hirano, M. and Yanagihara, T., Neurology 1996. 46: 791-5.
30 Barbanti, P., Fabbrini, G., Ricci, A., Cerbo, R., Bronzetti, E., Caronti, B., Calderaro, C., Felici, L., Stocchi, F., Meco, G., Amenta, F. and Lenzi, G. L., Mov Disord 1999.14: 764-71.
31 Barbanti, P., Fabbrini, G., Ricci, A., Bruno, G., Cerbo, R., Bronzetti, E., Amenta, F., Luigi Lenzi, G., Barbanti, P., Fabbrini, G., Ricci, A., Pascali, M. P., Bronzetti, E., Amenta, F., Lenzi, G. L. and Cerbo, R., Mech Ageing Dev 2000. 120: 65-75.
32 Barbanti, P., Bronzetti, E., Ricci, A., Cerbo, R., Fabbrini, G., Buzzi, M. G., Amenta, F. and Lenzi, G. L., Neurosci Lett 1996. 207: 73-6.
33 Barbanti, P., Fabbrini, G., Ricci, A., Pascali, M. P., Bronzetti, E., Amenta, F., Lenzi, G. L. and Cerbo, R., Cephalalgia 2000. 20: 15-9.
34 Diamond, M. S. and Springer, T. A., Curr-Biol 1994. 4: 506-17.
35 Springer, T. A., Cell 1994. 76: 301-14.
36 Clark, E. A. and Brugge, J. S., Science 1995. 268: 233-9.
37 and, Eur J Pharmacol 1995. 272: R1-3.
38 Levite, M., Cahalon, L., Hershkoviz, R., Steinman, L. and Lider, O., J-Inmmunol 1998.160: 993-1000.
39 Levite, M., Proc Natl Acad Sci USA 1998. 95: 12544-12549.
40 Kling-Petersen, T., Ljung, E., Waters, N. and Svensson, K., J Neural Transm 1996. 103: 331-41.
41 Waters, N., Svensson, K., Haadsma-Svensson, S. R., Smith, M. W. and Carlsson, A., J Neural Transm Gen Sect 1993. 94: 11-9.
42 Sokoloff, P., Giros, B., Martres, M. P., Bouthenet, M. L. and Schwartz, J. C., Nature 1990. 34 7: 146-51.
43 Fishburn, C. S., Belleli, D., David, C., Carmon, S. and Fuchs, S., J Biol Chem 1993. 268: 5872-8.
44 Levant, B., Pharmacol Rev 1997. 49: 231-52.
45 Fishburn, C. S. Bedford, M., Lonai, P., and Fuchs, S. Febs Lett 1996. 381: 257-61.
46 Scarselli, M., Novi, F., Schallmach, E., Lin, R., Baragli, A., Colzi, A. Griffon, N., Corsini, G. U., Sokoloff, P., Levenson, R., Vogel, Z. and Maggio, R., J Biol Chem 2001.
47 Rocheville, M., Lange, D. C., Kumar, U., Patel, S. C., Patel, R. C. and Patel, Y. C. Science 2000. 288: 154-7.
48 Guillin, O., Diaz, J., Carroll, P., Griffon, N., Schwartz, J. C. and Sokoloff, P. Nature 2001. 411: 86-9.
49 Besser, M. and Wank, R. J Immunol 1999. 162: 6303-6.

1. A method of regulating activity of a T-cell population, the method comprising exposing the T-cell population with a molecule selected capable of regulating a Dopamine receptor activity or the expression of a gene encoding a Dopamine receptor of T-cells of the T-cell population, thereby regulating Dopamine mediated activity in the T-cell population.

2. The method of claim 1, wherein the T-cell population is a resting T-cell population.

3. The method of claim 1, wherein said Dopamine receptor is a D3 Dopamine receptor.

4. The method of claim 1, wherein said molecule is selected capable of upregulating said Dopamine receptor activity or said expression of said gene encoding said Dopamine receptor, thereby upregulating Dopamine mediated activity of said T-cells of the T-cell population.

5. The method of claim 4, wherein said molecule is selected from the group consisting of Dopamine, an upregulating Dopamine analog, an upregulating anti Dopamine receptor antibody and an expressible polynucleotide encoding a Dopamine receptor.

6. The method of claim 5, wherein said upregulating anti-Dopamine receptor antibody is a monoclonal or a polyclonal antibody.

7. The method of claim 5, wherein said expressible polynucleotide encoding a Dopamine receptor is designed capable of transient expression within cells of the T-cell population.

8. The method of claim 5, wherein said expressible polynucleotide encoding a Dopamine receptor is designed capable of stably integrating into a genome of cells of the T-cell population.

9. The method of claim 5, wherein said expressible polynucleotide includes a sequence as set forth in any of SEQ ID NOs: 10 to 14.

10. The method of claim 1, wherein said molecule is selected capable of downregulating said Dopamine receptor activity or said expression of said gene encoding said Dopamine receptor, thereby downregulating Dopamine mediated activity in the T-cell population.

11. The method of claim 10 wherein said molecule is selected from the group consisting of a downregulating Dopamine analog, a downregulating anti Dopamine receptor antibody, a single stranded polynucleotide designed having specific Dopamine receptor transcript cleaving capability, an expressible polynucleotide encoding a ribozyme designed having specific Dopamine receptor transcript cleaving capability, a polynucleotide designed comprising nucleotide sequences complementary to, and capable of binding to Dopamine receptor transcripts, coding sequences and/or promoter elements and an expressible polynucleotide encoding nucleotide sequences complementary to, and capable of binding to Dopamine receptor transcripts, coding sequences and/or promoter elements.

12. The method of claim 11, wherein said downregulating anti-Dopamine receptor antibody is a monoclonal or a polyclonal antibody.

13. The method of claim 11, wherein said expressible polynucleotide is designed capable of transient expression within cells of the T-cell population.

14. The method of claim 11, wherein said expressible polynucleotide is designed capable of stably integrating into a genome of cells of the T-cell population.

15. The method of claim 11, wherein said expressible polynucleotide includes a sequence as set forth in any of SEQ ID NOs: 10-14.

16. The method of claim 1, wherein regulating Dopamine mediated activity in the T cell population results in a change in at least one T cell activity selected from the group consisting of β-integrin binding, fibronectin adhesion, depolarization, cytokine secretion, proliferation, gene expression and induction of inflammatory disease.

17. The method of claim 1, further comprising the step of monitoring said at least one T-cell activity in the T-cell population.

18. The method of claim 17, wherein said monitoring said at least one T-cell activity is effected by determining at least one parameter selected from the group consisting of β-integrin binding, fibronectin adhesion, depolarization, cytokine secretion, proliferation, gene expression and induction of inflammatory disease.

19. A method of suppressing activity of a T-cell population, the method comprising exposing the T-cell population with a concentration of a molecule selected capable of upregulating a Dopamine receptor activity, said concentration sufficient to suppress T-cell function in the T-cell population.

20. The method of claim 19, wherein said molecule selected capable of upregulating a Dopamine receptor activity is Dopamine or a Dopamine analog and whereas said concentration sufficient to suppress T-cell function is greater than 10⁻⁴ M.

21. The method of claim 19, wherein said Dopamine receptor is a D3 Dopamine receptor.

22. A method of regulating T-cell activity in a mammalian subject having abnormal T-cell activity, the method comprising providing to a subject identified as having the abnormal T-cell activity a therapeutically effective amount of a molecule selected capable of regulating a Dopamine receptor activity or an expression of a gene encoding said Dopamine receptor thereby regulating T-cell activity in the mammalian subject.

23. The method of claim 22, wherein the abnormal T-cell activity is suboptimal T-cell activity and whereas said molecule is selected capable of upregulating Dopamine receptor activity or said expression of said gene encoding said Dopamine receptor.

24. The method of claim 23, wherein said molecule selected capable of upregulating an activity of a Dopamine receptor or an expression of a gene encoding said dopamine receptor is selected from the group consisting of Dopamine, an upregulating Dopamine analog, an upregulating anti Dopamine receptor antibody and an expressible polynucleotide encoding a Dopamine receptor.

25. The method of claim 24, wherein said upregulating anti-Dopamine receptor antibody is a monoclonal or a polyclonal antibody.

26. The method of claim 24, wherein said expressible polynucleotide encoding a Dopamine receptor is designed capable of transient expression within cells of the subject.

27. The method of claim 24, wherein said expressible polynucleotide encoding a Dopamine receptor is designed capable of stably integrating into a genome of cells of the subject.

28. The method of claim 24, wherein said expressible polynucleotide includes a sequence as set forth in any of SEQ ID NOs: 10-14.

29. The method of claim 22 wherein said abnormal T-cell activity is excessive T-cell activity and whereas said molecule is selected capable of downregulating Dopamine receptor activity or said expression of said gene encoding said Dopamine receptor.

30. The method of claim 29 wherein said molecule selected capable of downregulating an activity of a Dopamine receptor or an expression of a gene encoding said dopamine receptor is selected from the group consisting of a downregulating Dopamine analog, a downregulating anti Dopamine receptor antibody, a single stranded polynucleotide designed having specific Dopamine receptor transcript cleaving capability, an expressible polynucleotide encoding a ribozyme designed having specific Dopamine receptor transcript cleaving capability, a polynucleotide designed comprising nucleotide sequences complementary to, and capable of binding to Dopamine receptor transcripts, coding sequences and/or promoter elements and an expressible polynucleotide encoding nucleotide sequences complementary to, and capable of binding to Dopamine receptor transcripts, coding sequences and/or promoter elements.

31. The method of claim 30, wherein said downregulating anti-Dopamine receptor antibody is a monoclonal or a polyclonal antibody.

32. The method of claim 30, wherein said expressible polynucleotide is designed capable of transient expression within cells of the subject.

33. The method of claim 30, wherein said expressible polynucleotide is designed capable of stably integrating into a genome of cells of the subject.

34. The method of claim 30, wherein said expressible polynucleotide includes a sequence as set forth in any of SEQ ID NOs: 10-14.

35. The method of claim 22, wherein said step of providing said molecule is effected by systemic or local administration of said molecule to the subject.

36. The method of claim 22, wherein said step of providing said molecule is effected by providing said molecule to an ex-vivo T-cell population and administering said ex-vivo T-cell population to the subject.

37. The method of claim 22, wherein the regulating T-cell activity in a mammalian subject results in a change in at least one T-cell activity selected from the group consisting of 0-integrin binding, fibronectin adhesion, depolarization, cytokine secretion, proliferation, gene expression and induction of inflammatory disease.

38. The method of claim 37, further comprising the step of monitoring a T-cell activity in T-cells of the subject.

39. The method of claim 38, wherein said monitoring said T-cell activity is effected by determining an activity selected from the group consisting of 0-integrin binding, fibronectin adhesion, depolarization, cytokine secretion, proliferation, gene expression and induction of inflammatory disease.

40. A method of treating or preventing a T-cell related disease or condition characterized by abnormal T-cell activity in a mammalian subject, the method comprising providing to a subject identified as having the T-cell related disease or condition characterized by abnormal T-cell activity a therapeutically effective amount of a molecule selected capable of regulating an activity of a Dopamine receptor or an expression of a gene encoding said Dopamine receptor, said amount being sufficient to regulate T-cell activity, thereby treating or preventing the T-cell related disease or condition in the mammalian subject.

41. The method of claim 40, wherein the T-cell related disease or condition is a disease or condition characterized by suboptimal T-cell activity selected from the group consisting of congenital immune deficiencies, acquired immune deficiencies, infection, neurological disease and injury, psychopathology and neoplastic disease; and whereas said molecule is selected capable of upregulating an activity of a Dopamine receptor or an expression of a gene encoding said dopamine receptor.

42. The method of claim 40, wherein said molecule selected capable of upregulating an activity of a Dopamine receptor or an expression of a gene encoding said dopamine receptor is selected from the group * consisting of Dopamine, an upregulating Dopamine analog, an upregulating anti Dopamine receptor antibody and an expressible polynucleotide encoding a Dopamine receptor.

43. The method of claim 42, wherein said upregulating anti-Dopamine receptor antibody is a monoclonal or a polyclonal antibody.

44. The method of claim 42, wherein said expressible polynucleotide encoding a Dopamine receptor is designed capable of transient expression within cells of the subject.

45. The method of claim 42, wherein said expressible polynucleotide encoding a Dopamine receptor is designed capable of stably integrating into a genome of cells of the subject.

46. The method of claim 42, wherein said expressible polynucleotide includes a sequence as set forth in any of SEQ ID NOs: 10-14.

47. The method of claim 40, wherein the T-cell related disease or condition is a disease or condition characterized by excessive T-cell activity selected from the group consisting of autoimmune, allergic, neoplastic, hyperreactive, pathopsychological and neurological diseases and conditions, graft-versus-host disease, and allograft rejections and whereas said molecule is selected capable of downregulating an activity of a Dopamine receptor or an expression of a gene encoding said dopamine receptor.

48. The method of claim 47, wherein said molecule selected capable of downregulating an activity of a Dopamine receptor or an expression of a gene encoding said dopamine receptor is selected from the group consisting of a downregulating Dopamine analog, a downregulating anti Dopamine receptor antibody, a single stranded polynucleotide designed having specific Dopamine receptor transcript cleaving capability, an expressible polynucleotide encoding a ribozyme designed having specific Dopamine receptor transcript cleaving capability, a polynucleotide designed comprising nucleotide sequences complementary to, and capable of binding to Dopamine receptor transcripts, coding sequences and/or promoter elements and an expressible polynucleotide encoding nucleotide sequences complementary to, and capable of binding to Dopamine receptor transcripts, coding sequences and/or promoter elements.

49. The method of claim 47, wherein said downregulating anti-Dopamine receptor antibody is a monoclonal or a polyclonal antibody.

50. The method of claim 47, wherein said expressible polynucleotide is designed capable of transient expression within cells of the subject.

51. The method of claim 47, wherein said expressible polynucleotide is designed capable of stably integrating into a genome of cells of the subject.

52. The method of claim 47, wherein said expressible polynucleotide includes a sequence as set forth in any of SEQ ID NOs: 10-14.

53. The method of claim 40, wherein said step of providing said molecule is effected by systemic or local administration of said molecule to the subject.

54. The method of claim 40, wherein said step of providing said molecule is effected by providing said molecule to an ex-vivo T-cell population and administering said ex-vivo T-cell population to the subject.

55. The method of claim 40, wherein the regulating T-cell activity in a mammalian subject results in a change in at least one T-cell activity selected from the group consisting of P-integrin binding, fibronectin adhesion, depolarization, cytokine secretion, proliferation, gene expression and induction of inflammatory disease.

56. The method of claim 55, further comprising the step of monitoring a T-cell activity in T-cells of the subject.

57. The method of claim 56, wherein monitoring said T-cell activity is effected by determining an activity selected from the group consisting of, β-integrin binding, fibronectin adhesion, depolarization, cytokine secretion, proliferation, gene expression and induction of inflammatory disease.

58. The method of claim 40 wherein the subject is suffering from a cancerous disease or condition characterized by excess T-cell activity, and whereas the method further comprising the step of determining cancer cell proliferation and/or metastasis in the subject prior to and/or following said step of providing.

59. The method of claim 58, wherein said cancerous disease or condition characterized by excess T-cell activity is a myeloproliferative disease.

60. The method of claim 40, wherein the T-cell related disease or condition is a T-cell inflammatory disease or condition characterized by excessive T-cell activity, and whereas said molecule is a molecule selected capable of upregulating an activity of a Dopamine receptor, further comprising the step of exposing stimulated T cells from the subject to a therapeutically effective amount of said molecule selected capable of upregulating an activity of a Dopamine receptor, thereby suppressing said T cell inflammatory disease in the subject.

61. The method of claim 60, further comprising the step of monitoring a symptom of said T-cell inflammatory disease or condition in the subject prior to and/or following said step of providing.

62. The method of claim 60, wherein said T-cell inflammatory disease is selected from the group consisting of Delayed Type Hypersensitivity (DTH), Experimental Autoimmune Encephalomyelitis (EAE) and Multiple Sclerosis (MS).

63. A population of T-cells suitable for treating or preventing a disease or condition characterized by abnormal T-cell activity in a subject, the population of T cells comprising T-cells characterized by modified sensitivity to Dopamine receptor stimulation, said T-cells being capable of treating or preventing a disease or condition characterized by abnormal T-cell activity upon administration to the subject.

64. The population of T-cells of claim 63, wherein said T-cells comprise an exogenous expressible polynucleotide sequence encoding expressing a Dopamine receptor.

65. The population of T-cells of claim 63, wherein said T-cells comprise an exogenous polynucleotide sequence capable of downregulating expression of a gene encoding a Dopamine receptor.

66. An assay for determining the sensitivity of a resting T-cell population to regulation of Dopamine receptor activity, the assay comprising:
(a) exposing the T-cell population to a molecule selected capable of regulating a Dopamine receptor activity or the expression of a gene encoding a Dopamine receptor, and
(b) assessing a state of the T-cell population.

67. The assay of claim 66 wherein step (a) is effected by exposing the T-cell population to a range of concentrations of said molecule, and whereas step (b) is effected by assessing said state at each concentration of said range.

68. The assay of claim 66 wherein said Dopamine receptor is a D3 Dopamine receptor.

69. The assay of claim 66, wherein said molecule is a molecule selected capable of upregulating said Dopamine receptor activity or said expression of said gene encoding said Dopamine receptor, thereby upregulating Dopamine mediated activity in the T-cell population.

70. The assay of claim 69, wherein said molecule selected capable of upregulating an activity of a Dopamine receptor or an expression of a gene encoding said dopamine receptor is selected from the group consisting of Dopamine, an upregulating Dopamine analog, an upregulating anti Dopamine receptor antibody and an expressible polynucleotide encoding a Dopamine receptor.

71. The assay of claim 70, wherein said upregulating anti-Dopamine receptor antibody is a monoclonal or a polyclonal antibody.

72. The assay of claim 70, wherein said expressible polynucleotide encoding a Dopamine receptor is designed capable of transient expression within cells of the T-cell population.

73. The assay of claim 70, wherein said expressible polynucleotide encoding a Dopamine receptor is designed capable of stably integrating into a genome of cells of the T-cell population.

74. The assay of claim 70, wherein said expressible polynucleotide includes a sequence as set forth in any of SEQ ID NOs: 10-14.

75. The assay of claim 66, wherein said molecule is a molecule selected capable of downregulating said Dopamine receptor activity or said expression of said gene encoding said Dopamine receptor, thereby downregulating Dopamine mediated activity in the T-cell population.

76. The assay of claim 75 wherein said molecule selected capable of downregulating an activity of a Dopamine receptor or an expression of a gene encoding said dopamine receptor is selected from the group consisting of a downregulating Dopamine analog, a downregulating anti Dopamine receptor antibody, a single stranded polynucleotide designed having specific Dopamine receptor transcript cleaving capability, an expressible polynucleotide encoding a ribozyme designed having specific Dopamine receptor transcript cleaving capability, a polynucleotide designed comprising nucleotide sequences complementary to, and capable of binding to Dopamine receptor transcripts, coding sequences and/or promoter elements and an expressible polynucleotide encoding nucleotide sequences complementary to, and capable of binding to Dopamine receptor transcripts, coding sequences and/or promoter elements.

77. The assay of claim 76, wherein said downregulating anti-Dopamine receptor antibody is a monoclonal or a polyclonal antibody.

78. The assay of claim 76, wherein said expressible polynucleotide is designed capable of transient expression within cells of the T-cell population.

79. The assay of claim 76, wherein said expressible polynucleotide is designed capable of stably integrating into a genome of cells of the T-cell population.

80. The assay of claim 76, wherein said expressible polynucleotide includes a sequence as set forth in any of SEQ ID NOs: 10-14.

81. The assay of claim 66, wherein step (b) is effected by determining an activity selected from the group consisting of β-integrin binding, fibronectin adhesion, depolarization, cytokine secretion, proliferation, gene expression and induction of inflammatory disease.

82. An article of manufacture, comprising packaging material and a therapeutically effective amount of a pharmaceutical composition being identified for the treatment of a T-cell related disease or condition associated with abnormal T-cell activity, said pharmaceutical composition including a molecule selected capable of regulating an activity of a Dopamine receptor or an expression of a gene encoding said Dopamine receptor in T-cells and a pharmaceutically acceptable carrier.

83. The article of manufacture of claim 82, wherein said molecule is capable of upregulating an activity of a Dopamine receptor or an expression of a gene encoding said Dopamine receptor in T-cells and whereas the T-cell related disease or condition is a disease or condition characterized by suboptimal T-cell activity.

84. The article of manufacture of claim 83, wherein said molecule selected capable of upregulating an activity of a Dopamine receptor or an expression of a gene encoding said dopamine receptor is selected from the group consisting of Dopamine, an upregulating Dopamine analog, an upregulating anti Dopamine receptor antibody and an expressible polynucleotide encoding a Dopamine receptor.

85. The article of manufacture of claim 82, wherein said molecule is capable of downregulating an activity of a Dopamine receptor or an expression of a gene encoding said Dopamine receptor in T-cells and whereas the T-cell related disease or condition is a disease or condition characterized by excessive T-cell activity.

86. The article of manufacture of claim 85, wherein said molecule selected capable of downregulating an activity of a Dopamine receptor or an expression of a gene encoding said dopamine receptor is selected from the group consisting of a downregulating Dopamine analog, a downregulating anti Dopamine receptor antibody, a single stranded polynucleotide designed having specific Dopamine receptor transcript cleaving capability, an expressible polynucleotide encoding a ribozyme designed having specific Dopamine receptor transcript cleaving capability, a polynucleotide designed comprising nucleotide sequences complementary to, and capable of binding to Dopamine receptor transcripts, coding sequences and/or promoter elements and an expressible polynucleotide encoding nucleotide sequences complementary to, and capable of binding to Dopamine receptor transcripts, coding sequences and/or promoter elements.
